(19) United States
(12) Patent Application Publication
(10) Pub. No.: US 2026/0317292 A1
(43) Pub. Date: Oct. 15, 2026

(54) SMALL MOLECULE THERAPY FOR DUCHENNE MUSCULAR DYSTROPHY

(71) Applicant: The Regents of the University of California, Oakland, CA (US)

(72) Inventors: Varghese John, Los Angeles, CA (US); Rachelle H. Crosbie, Los Angeles, CA (US); Ekaterina Mokhonova, Los Angeles, CA (US); Jesus J. Campagna, Los Angeles, CA (US); Barbara Jagodzinska, Redwood City, CA (US); Whitaker Cohn, Los Angeles, CA (US); Ravinder Malik, Los Angeles, CA (US); Dongwook Wi, Los Angeles, CA (US)

(21) Appl. No.: 19/479,880

(22) PCT Filed: May 2, 2024
(86) PCT No.: PCT/US2024/027387

§ 371 Date: Oct. 29, 2025

Related U.S. Application Data
(60) Provisional application No. 63/463,662, filed on May 3, 2023.

(51) Int. Cl. C07D 311/14; A61K 31/551; A61K 31/553; A61P 21/00; C07D 215/54; C07D 311/16; C07D 401/12; C07D 471/04

(52) U.S. Cl. CPC C07D 311/14; A61K 31/551; A61K 31/553; A61P 21/00; C07D 215/54; C07D 311/16; C07D 401/12; C07D 471/04

(57) ABSTRACT

The present disclosure relates to compounds that increase sarcospan expression. The disclosure further relates to methods of treating a disease related to diminution or dysfunction of a dystrophin-related complex in a subject in need thereof.

### CROSS-REFERENCE TO RELATED APPLICATIONS

This application claims the benefit of U.S. Provisional Patent Application No. 63/463,662, filed on May 3, 2023, which is hereby incorporated by reference in its entirety.

### GOVERNMENT SUPPORT

This invention was made with government support under AR048179 awarded by the National Institutes of Health. The government has certain rights in the invention.

### BACKGROUND

Duchenne muscular dystrophy (DMD) is an inherited muscle wasting disease that affects 1:5,500 males at birth. As of 2017, the only FDA approved drugs for DMD minimally slow progression of the disease and/or only target a fraction of the affected population.

Sarcospan-based therapies can treat the entire DMD population regardless of specific mutation. Therapeutic interventions for increasing sarcospan expression are needed.

### SUMMARY OF THE INVENTION

In certain aspects, the present disclosure provides a compound represented by formula (I) or a pharmaceutically acceptable salt thereof:

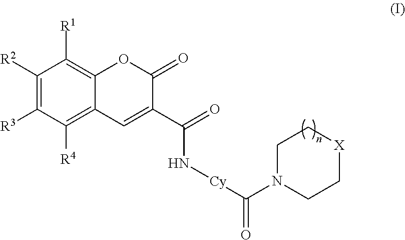

wherein
R¹, R², R³, and R⁴ are each independently H, alkyl, alkoxy, halo, nitrile, nitro, or amino;
Cy is aryl or heteroaryl;
n is 1, or 2; and
X is O or NH.

In further aspects, the present disclosure provides a compound represented by formula (II) or a pharmaceutically acceptable salt thereof:

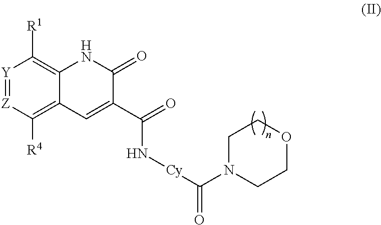

wherein
R¹ and R⁴ are each independently H, alkyl, alkoxy, halo, nitrile, nitro, amino or aminoalkyl;
Y is N or CR²;
R² is H, halo, nitro, amino, alkoxy, or alkyl;
Z is N or CR³;
R³ is H, halo, nitro, amino, alkoxy, or alkyl;
Cy is aryl or heteroaryl; and
n is 1 or 2; and
X is O or NH; wherein the compound is not

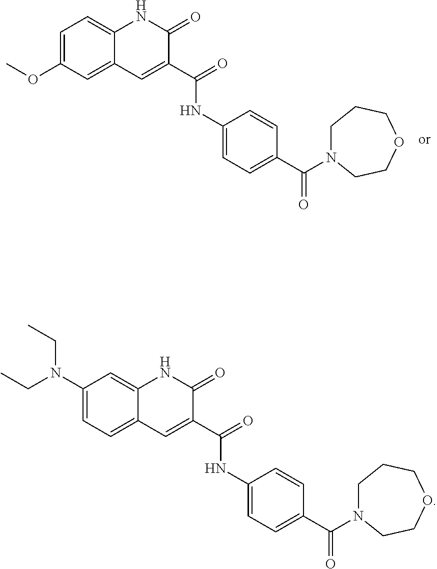 [SMILES: COc1ccc2[nH]c(=O)c(C(=O)Nc3ccc(C(=O)N4CCCOCC4)cc3)cc2c1 ; CCN(CC)c1ccc2cc(C(=O)Nc3ccc(C(=O)N4CCCOCC4)cc3)c(=O)[nH]c2c1]

### BRIEF DESCRIPTION OF THE DRAWINGS

FIG. 1. Activity of DDL-463, DDL-465 (A) and DDL-472 (B) in HiBiT assay. Comparison of activity of the new compounds with parent compound (OT-9m) and vehicle-treated cells. SSPN-HiBiT myotubes were treated on day 2 and harvested on day 4 of differentiation. R.U.=relative units normalized to protein concentration and vehicle control (DMSO). n=18. **p<0.01, ****p<0.0001, ns-difference is non-significant from the vehicle-treated cells.

FIG. 2. Concentration of DDL-465, DDL-472 and OT-9m in plasma and muscles after oral gavage. Concentrations of DDL-465, DDL-472 and OT-9m (DDL-449) in mice plasma and muscle (gastrocnemius) samples after oral gavage were analyzed through the LC-MS/MS. The mean+/−SD values are plotted on the time scale. Right graph is zoomed in fragment of the left graph with OT-9m and DDL-465.

FIG. 3. Single administration of 60 mg/kg of DDL-472 by oral gavage increases level of SSPN transcript in C57BL/6 mice by 1.7-fold at 2 hours post-administration.

Tissues after 2, 4 and 24 hours post administration were analyzed. Gene expression was normalized to β-actin, mouse weight and SSPN transcript level in vehicle-treated mice. Mean value of SSPN mRNA in vehicle-treated group is represented by dash line. A. SSPN mRNA level over 24-hours period after oral gavage. SSPN mRNA level in vehicle-treated group is presented as time “0”. Data represents mean value per treated group and SD. B. All replicates from animals. Data represents individual replicates, mean value per treated group and SD. ***p<0.001, *p<0.5.

FIG. 4. DDL-472 increase SSPN gene expression in TAs of mdx mice after local administration. 6-weeks old mdx mice were injected locally in TA muscle with 0.25 mg/kg of DDL-472. Muscles were harvested after 4 hours post-injections and analyzed by qPCR. Gene expression was normalized to β-actin, mouse weight and SSPN transcript level in vehicle-treated mice. Data represents individual replicates, mean value and SD. ***p<0.001, *p<0.5. 0.25 mg/kg corresponds to 5-10 uM of local concentration of DDL-472 in TA (active concentration of DDL-472 in vitro).

FIG. 5. De-escalation of DDL-472. (A) concentrations of DDL-472 in gastrocnemius muscles and plasma. (B) Levels of SSPN mRNA after de-escalation of DDL-472. DDL-472 (0.2-60 MKD) was given to C57 BL/6 mice by oral gavage. Tissues after 2 hours post administration were harvested for PK and qPCR analysis. Concentrations of compound in gastrocnemius and plasma were analyzed through the LC-MS/MS. Gene expression was normalized to β-actin, mice weight and SSPN transcript level in vehicle-treated mice. Data represents individual replicates, mean value per treated group and SD. ***p<0.001.

FIG. 6. DDL-472 increase SSPN and β-Integrin gene expression in skeletal muscles of mdx mice after systemic administration. Tissues were harvested on day 22 at 2 hours after last compound administration. Gene expression was normalized to β-actin, mouse weight and β-Integrin transcript level in vehicle-treated mice. Three non-responders from the compound-treated group are removed on SSPN mRNA plot. Data represents individual replicates, mean value per treated group and SD. Mean value of SSPN and β-Integrin mRNA in vehicle-treated group is represented by dash line. **p<0.01.

FIG. 7. DDL-472 increases sarcolemma expression of SSPN and integrin 31 proteins in compound-treated mice. 10 μm quadriceps cryo-sections were stained with SSPN rabbit mAb10B8 (Crosbie Lab) and β-Integrin (EMD Millipore #MAB1900).

FIG. 8. DDL-449 increase SSPN gene expression in skeletal muscles (A) and hearts of mdx mice (B) after systemic oral administration. Gene expression was normalized to β-actin, mouse weight and sarcospan transcript level in vehicle-treated mice. Data represents individual replicates, mean value per treated group and SD. Mean value of SSPN mRNA in vehicle-treated group is represented by dash line. ****p<0.0001.

FIG. 9. DDL-449 increases sarcolemma expression of SSPN, Utrophin and integrin 1 proteins in compound-treated mice. 10 μm quadriceps cryo-sections were stained with SSPN rabbit mAb10B8 (Crosbie Lab), Utrophin MANCHO3 (DSHB) and β-Integrin (EMD Millipore #MAB1900). Scale bar—50 μm.

FIG. 10. New quinolone analogues DDL-494, DDL-495, DDL-496 are active in vitro in HiBiT assay.

Comparison of activity of the new compounds of quinolone series (DDL-494, DDL-495, DDL-496) with parent compound (DDL-449 (aka OT-9m)) and vehicle-treated cells. SSPN-HiBiT myotubes were treated on day 2 and harvested on day 4 of differentiation. R.U.=relative units normalized to protein concentration and vehicle control (DMSO). n=18 for every compound concentration. *p<0.05; **p<0.01, ***p<0.001; ****p<0.0001

FIG. 11. New coumarin analogue DDL-491 is active in vitro in HiBiT assay.

Comparison of activity of the new compound of coumarin series (DDL-491) with parent compound (DDL-472) and vehicle-treated cells. SSPN-HiBiT myotubes were treated on day 2 and harvested on day 4 of differentiation. R.U.=relative units normalized to protein concentration and vehicle control (DMSO). n=18 for every compound concentration. *p<0.05; ****p<0.0001

FIG. 12. Determination of EC₅₀ for DDL-494 and parent compound DDL-449 on Mouly DMD myotubes.

Mouly DMD myotubes were treated with DDL-494 and DDL-449 (5000, 2500, 1250, 625, 312, and 156 nM) on day 2 of differentiation and harvested on day 4 of differentiation. Gene expression was normalized to housekeeping gene β-actin and vehicle-treated cells (0.5% DMSO). SSPN gene expression is represented as percentage increase normalized to the starting value. Data represent mean values and standard error, n=9 for every concentration of the compounds.

FIG. 13. Determination of EC₅₀ for DDL-494 and parent compound DDL-449 on H2K mdx myotubes.

H2K mdx myotubes were treated with DDL-494 and DDL-449 (5000, 2500, 1250, 625, 312, and 156 nM) on day 2 of differentiation and harvested on day 4 of differentiation. Gene expression was normalized to housekeeping gene β-actin and vehicle-treated cells (0.5% DMSO). SSPN gene expression is represented as percentage increase normalized to the starting value. Data represent mean values and standard error, n=9 for every concentration of the compounds.

FIG. 14. Determination of EC₅₀ for DDL-491 and parent compound DDL-472 on Mouly DMD myotubes.

Mouly DMD myotubes were treated with DDL-491 and DDL-472 (5000, 2500, 1250, 625, 312, and 156 nM) on day 2 of differentiation and harvested on day 4 of differentiation. Gene expression was normalized to housekeeping gene β-actin and vehicle-treated cells (0.5% DMSO). SSPN gene expression is represented as percentage increase normalized to the starting value. Data represent mean values and standard error, n=9 for every concentration of the compounds.

FIG. 15. Determination of EC₅₀ for DDL-491 and parent compound DDL-472 on H2K mdx myotubes.

H2K mdx myotubes were treated with DDL-491 and DDL-472 (5000, 2500, 1250, 625, 312, and 156 nM) on day 2 of differentiation and harvested on day 4 of differentiation. Gene expression was normalized to housekeeping gene β-actin and vehicle-treated cells (0.5% DMSO). SSPN gene expression is represented as percentage increase normalized to the starting value. Data represent mean values and standard error, n=9 for every concentration of the compounds.

FIG. 16. Treatment by 20 MKD of DDL-472 twice weekly for 3 weeks increases SSPN and β-Integrin transcripts in skeletal muscles.

mdx mice (9-11 weeks of age) were treated by 20 MKD of DDL-472 for 3 weeks twice weekly by pipet feeding. Muscle tissues were harvested on day 22 for analysis. (A). SSPN mRNA analysis in skeletal muscles by qPCR. Gene expression was normalized to β-actin, mouse weight and SSPN transcript level in vehicle-treated mice. Data represents individual replicates, mean value per treated group and SD. Mean value of SSPN mRNA in vehicle-treated group is represented by green line. *p<0.05. (B). Gene expression was normalized to β-actin, mouse weight and b-Integrin transcript level in vehicle-treated mice. Data represents individual replicates, mean value per treated group and SD. Mean value of SSPN mRNA in vehicle-treated group is represented by dash line. **p<0.01.

FIG. 17. Bi-weekly dosing by 20 MKD of DDL-472 for 3 weeks increases SSPN protein in skeletal muscles.

mdx mice (9-11 weeks of age) were treated by 20 MKD of DDL-472 for 3 weeks twice weekly by pipet feeding. Muscle tissues were harvested on day 22 for analysis. (A). Immunoblotting of SSPN protein in total lysate of skeletal muscles. Ponceau S-stained membrane is shown for total protein loading control. (B). Densitometry analysis of immunoblotting results presented in panel A. Densitometry data were normalized to total loading by Ponceau S and SSPN protein level in vehicle-treated mice. Each data point represents individual mouse, mean value per treated group and SD. *p<0.05

### DETAILED DESCRIPTION OF THE INVENTION

The heterogeneity of mutations and difficulty of delivery to muscle are major challenges to the development of therapies to treat DMD. There is an urgent need for improved therapies that can overcome these challenges. Sarcospan (SSPN) reduces the pathology of muscular dystrophy in the DMD murine model by increasing membrane localization of the utrophin-glycoprotein complex (UGC) and α7β1D-integrin adhesion complexes, effectively increasing laminin binding to compensate for the loss of dystrophin.

Development of small molecule therapies that increase SSPN expression may lead to standalone or combinatorial therapies to treat DMD and other forms of muscular dystrophy caused by deficits in membrane proteins. Small molecule therapies are ideal due to their ability to bypass the limitations of delivery and immune responses seen with viral and cell-based methods. Small molecule therapies that increase SSPN expression are disclosed in WO 2022/055926, the contents of which are included by reference herein.

Disclosed here are small molecules that increase expression of sarcospan for the treatment of Duchenne muscular dystrophy and other indications in which loss of cell attachment or cell membrane dysfunction is a component of the disease pathology.

In certain embodiments, the present disclosure provides a compound represented by formula (I) or a pharmaceutically acceptable salt thereof:

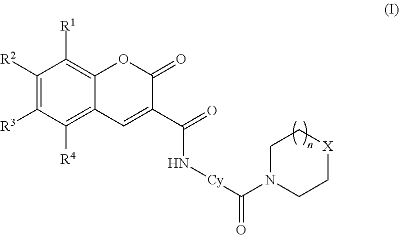

wherein
R¹, R², R³, and R⁴ are each independently H, alkyl, alkoxy, halo, nitrile, nitro, amino or aminoalkyl;
Cy is aryl or heteroaryl;
n is 1 or 2; and
X is O or NH.

In certain embodiments, Cy is aryl.

In certain embodiments, the compound is represented by formula (Ia):

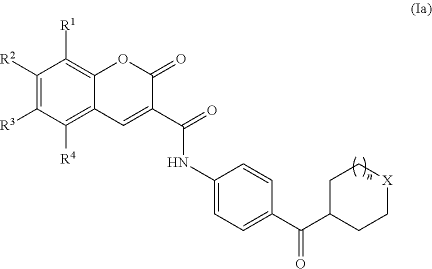

In certain embodiments, R¹ and R⁴ are H.

In certain embodiments, n is 2.

In certain embodiments, X is O.

In certain embodiments, R³ is H or halo, preferably H.

In certain embodiments, R² is H, halo, alkyl, alkoxy, amino, acetyl, or aminoalkyl. In certain such embodiments, R² is a cyclic amine. In certain other embodiments, R² is dimethylamino, diethylamino or di-isopropyl amino. In certain such embodiments, R² is diethylamino.

In certain embodiments, the compound is selected from:

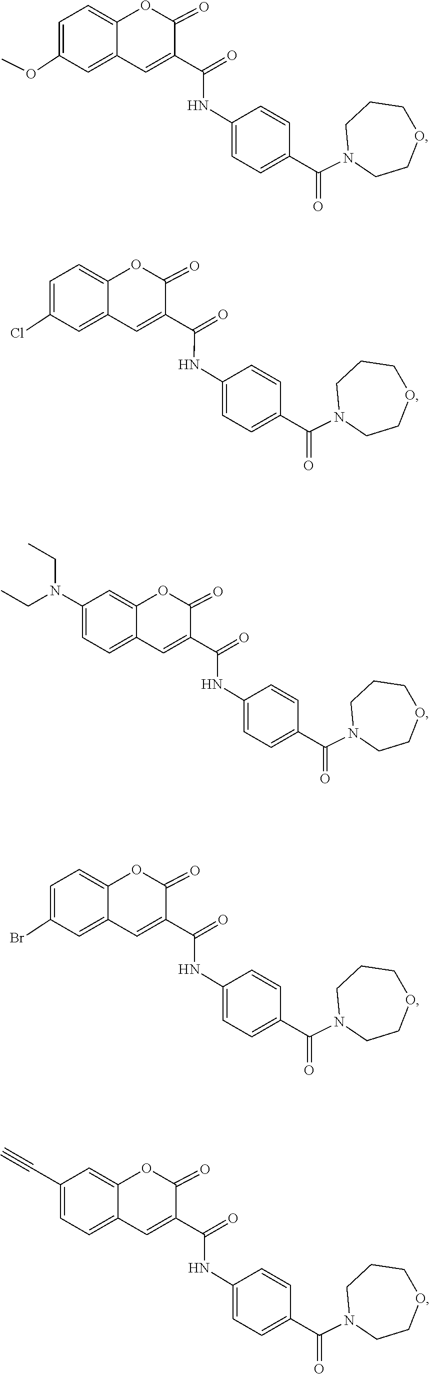 [SMILES: COc1ccc2oc(=O)c(C(=O)Nc3ccc(C(=O)N4CCCOCC4)cc3)cc2c1 ; O=C(Nc1ccc(C(=O)N2CCCOCC2)cc1)c1cc2cc(Cl)ccc2oc1=O ; CCN(CC)c1ccc2cc(C(=O)Nc3ccc(C(=O)N4CCCOCC4)cc3)c(=O)oc2c1 ; O=C(Nc1ccc(C(=O)N2CCCOCC2)cc1)c1cc2cc(Br)ccc2oc1=O ; C#Cc1ccc2cc(C(=O)Nc3ccc(C(=O)N4CCCOCC4)cc3)c(=O)oc2c1]

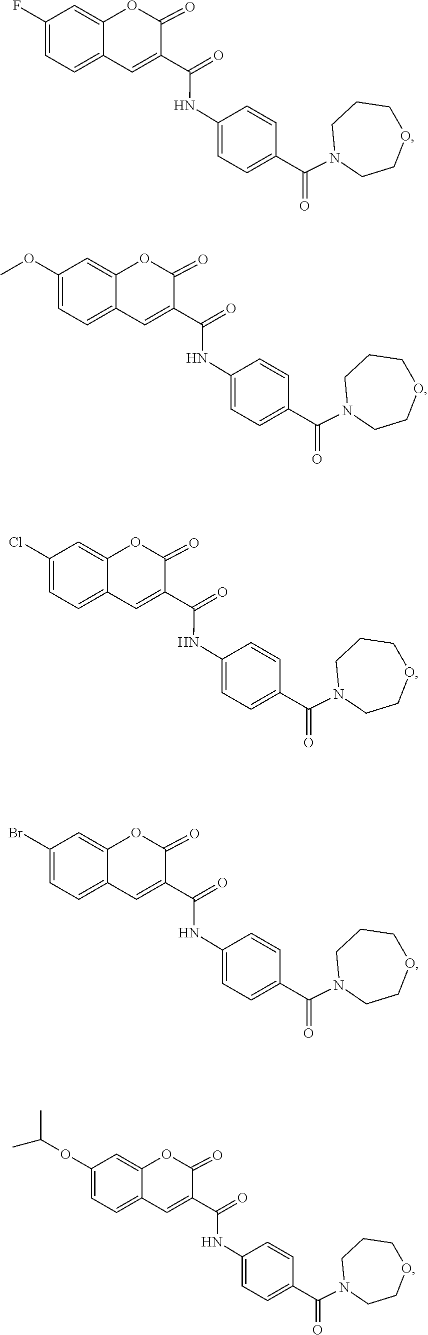 [SMILES: O=C(Nc1ccc(C(=O)N2CCCOCC2)cc1)c1cc2ccc(F)cc2oc1=O ; COc1ccc2cc(C(=O)Nc3ccc(C(=O)N4CCCOCC4)cc3)c(=O)oc2c1 ; O=C(Nc1ccc(C(=O)N2CCCOCC2)cc1)c1cc2ccc(Cl)cc2oc1=O ; O=C(Nc1ccc(C(=O)N2CCCOCC2)cc1)c1cc2ccc(Br)cc2oc1=O ; CC(C)Oc1ccc2cc(C(=O)Nc3ccc(C(=O)N4CCCOCC4)cc3)c(=O)oc2c1]

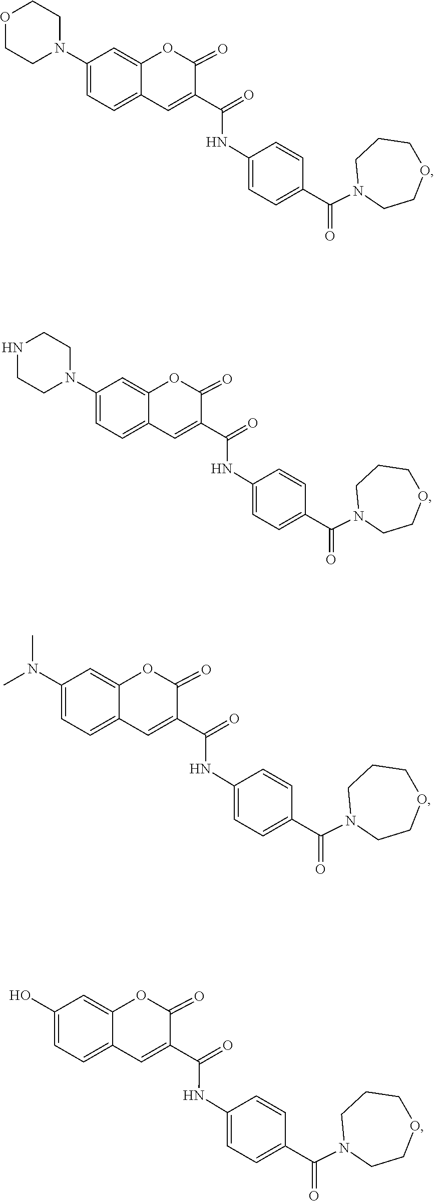 [SMILES: O=C(Nc1ccc(C(=O)N2CCCOCC2)cc1)c1cc2ccc(N3CCOCC3)cc2oc1=O ; O=C(Nc1ccc(C(=O)N2CCCOCC2)cc1)c1cc2ccc(N3CCNCC3)cc2oc1=O ; CN(C)c1ccc2cc(C(=O)Nc3ccc(C(=O)N4CCCOCC4)cc3)c(=O)oc2c1 ; O=C(Nc1ccc(C(=O)N2CCCOCC2)cc1)c1cc2ccc(O)cc2oc1=O]

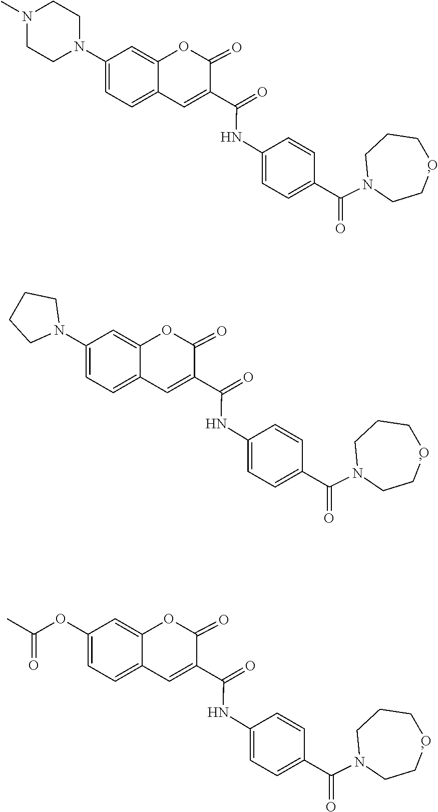 [SMILES: CN1CCN(c2ccc3cc(C(=O)Nc4ccc(C(=O)N5CCCOCC5)cc4)c(=O)oc3c2)CC1 ; O=C(Nc1ccc(C(=O)N2CCCOCC2)cc1)c1cc2ccc(N3CCCC3)cc2oc1=O ; CC(=O)Oc1ccc2cc(C(=O)Nc3ccc(C(=O)N4CCCOCC4)cc3)c(=O)oc2c1]

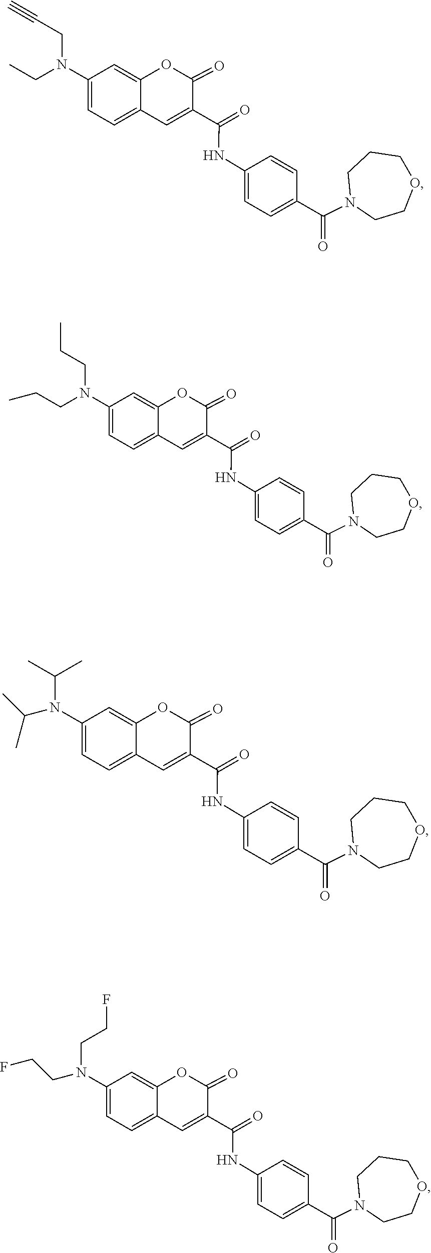 [SMILES: C#CCN(CC)c1ccc2cc(C(=O)Nc3ccc(C(=O)N4CCCOCC4)cc3)c(=O)oc2c1 ; CCCN(CCC)c1ccc2cc(C(=O)Nc3ccc(C(=O)N4CCCOCC4)cc3)c(=O)oc2c1 ; CC(C)N(c1ccc2cc(C(=O)Nc3ccc(C(=O)N4CCCOCC4)cc3)c(=O)oc2c1)C(C)C ; O=C(Nc1ccc(C(=O)N2CCCOCC2)cc1)c1cc2ccc(N(CCF)CCF)cc2oc1=O]

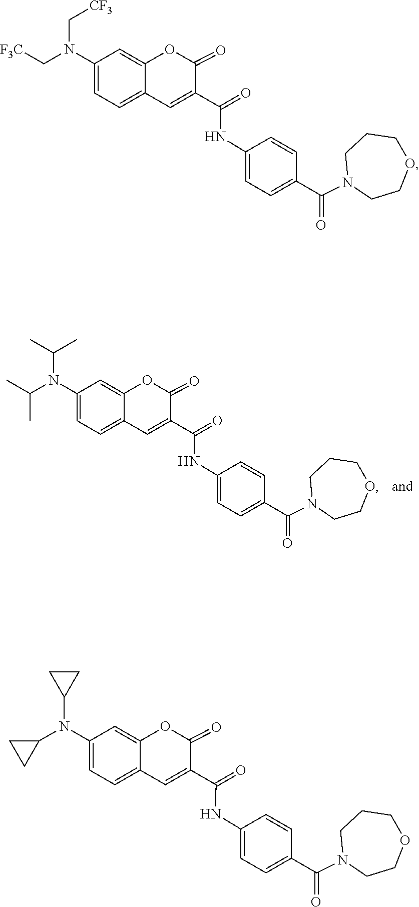 [SMILES: O=C(Nc1ccc(C(=O)N2CCCOCC2)cc1)c1cc2ccc(N(CC(F)(F)F)CC(F)(F)F)cc2oc1=O ; CC(C)N(c1ccc2cc(C(=O)Nc3ccc(C(=O)N4CCCOCC4)cc3)c(=O)oc2c1)C(C)C ; "and" ; O=C(Nc1ccc(C(=O)N2CCCOCC2)cc1)c1cc2ccc(N(C3CC3)C3CC3)cc2oc1=O]

or a pharmaceutically acceptable salt thereof.

In certain embodiments, the present disclosure provides a compound represented by formula (II) or a pharmaceutically acceptable salt thereof.

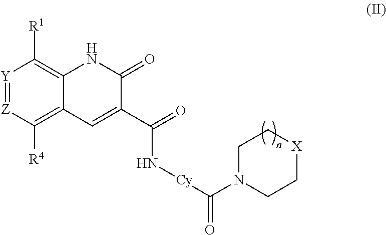

wherein
R¹ and R⁴ are each independently H, alkyl, alkoxy, halo, nitrile, nitro, amino or aminoalkyl;
Y is N or CR²;
R² is H, halo, nitro, amino, alkoxy, or alkyl;
Z is N or CR³;
R³ is H, halo, nitro, amino, alkoxy, or alkyl;
Cy is aryl or heteroaryl;
n is 1 or 2; and
X is O or NH; wherein the compound is not

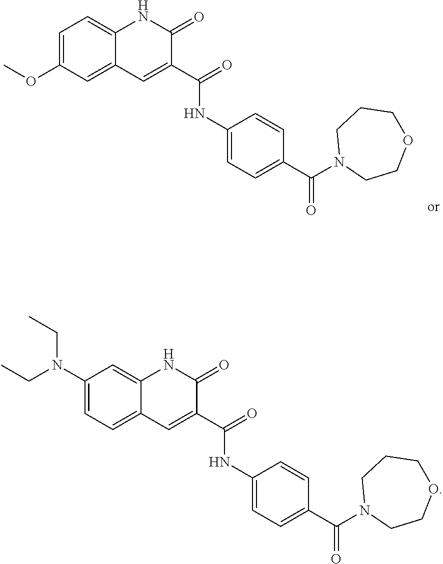 [SMILES: COc1ccc2[nH]c(=O)c(C(=O)Nc3ccc(C(=O)N4CCCOCC4)cc3)cc2c1 ; CCN(CC)c1ccc2cc(C(=O)Nc3ccc(C(=O)N4CCCOCC4)cc3)c(=O)[nH]c2c1]

In certain embodiments, Cy is aryl.

In certain embodiments, R¹ and R⁴ are H.

In certain embodiments, n is 2.

In certain embodiments, X is O.

In certain embodiments, R³ is H, alkyl, alkoxy, halo, amino, or nitro. In certain such embodiments, R³ is amino. In certain other embodiments, R³ is haloalkyl. In certain such embodiments, R³ is alkyl substituted with one or more F.

In certain embodiments, Y is CR². In certain such embodiments, R² is halo or alkoxy. In other embodiments, Y is N.

In certain embodiments, the compound is selected from:

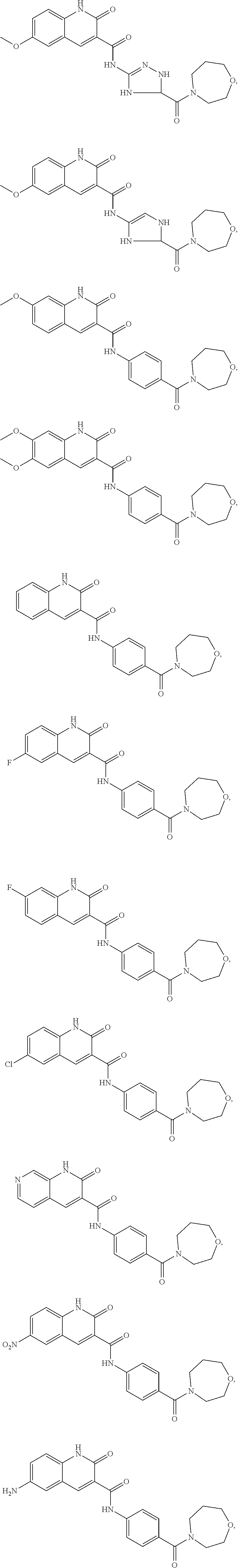

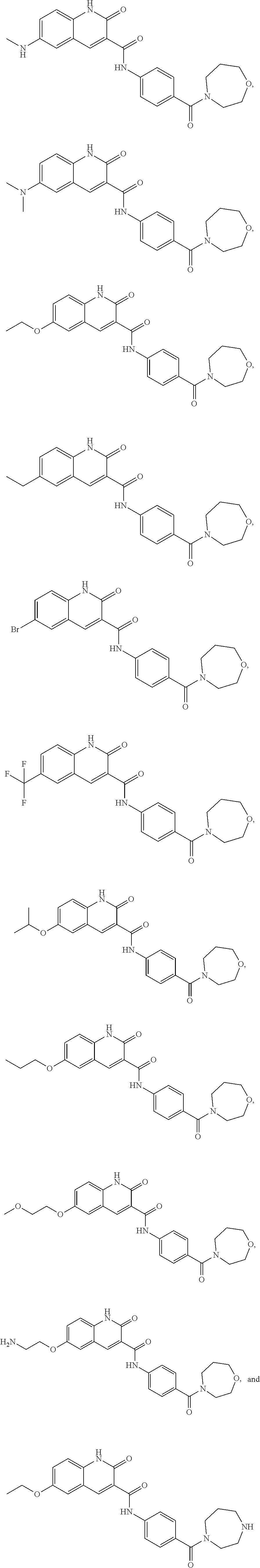

or a pharmaceutically acceptable salt thereof.

In certain embodiments, the invention relates to a pharmaceutical composition comprising a compound of formula (I) or formula (II) and a pharmaceutically available excipient.

In certain embodiments, the invention relates to a method of treating or preventing a disease related to dysfunction of a dystrophin-related complex in a subject in need thereof, comprising administering to the subject a compound formula (I) or formula (II). In certain such embodiments, the disease related to dysfunction of a dystrophin-related complex is muscular dystrophy. In certain such embodiments, the disease is Duchenne muscular dystrophy.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro-emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, l-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow-release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as a muscular dystrophy. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

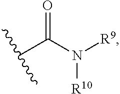

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

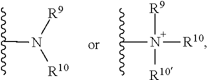

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

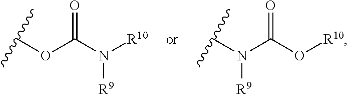

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

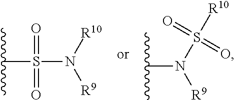

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

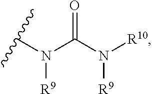

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Reaction Scheme by Batch Chemistry Synthesis (Quinolone Series)

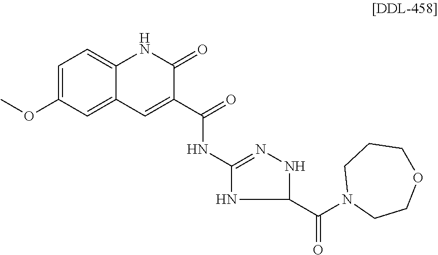 [SMILES: COc1ccc2[nH]c(=O)c(C(=O)NC3=NNC(C(=O)N4CCCOCC4)N3)cc2c1]

(3-amino-1H-1,2,4-triazol-5-yl)(1,4-oxazepan-4-yl)methanone (2.1). Step 1: To a mixture of 3-amino-1H-1,2,4-triazole-5-carboxylic acid (400 mg, 3.12 mmol), EDC (1.2 g, 6.25 mmol), and Hydroxybenzotriazole (598 mg, 3.12 mmol, 80% wt.) in dry DMF (2 mL) was added DIEA (1.63 mL, 9.67 mmol). The mixture was stirred at RT for 10 min followed by the addition of 1,4-oxazepane hydrochloride (516 mg, 3.75 mmol). The mixture was stirred at RT overnight and purified directly on a C18 flash column (40 g) eluted with acetonitrile and water (0 to 100%) to afford 114 mg (17%) of compound 2.1 as foamed solid. N-(5-(1,4-oxazepane-4-carbonyl)-1H-1,2,4-triazol-3-yl)-6-methoxy-2-oxo-1,2-dihydroquinoline-3-carboxamide (DDL-459). Step-2: To a mixture of 6-methoxy-2-oxo-1,2-dihydroquinoline-3-carboxylic acid (45 mg, 0.21 mmol) and HATU (120 mg, 0.31 mmol) in dry DMF (2 mL) was added DIEA (0.14 mL, 0.82 mmol). The mixture was stirred at RT for 10 min followed by the addition of (3-amino-1H-1,2,4-triazol-5-yl)(1,4-oxazepan-4-yl)methanone (87 mg, 0.31 mmol). The mixture was stirred at 40° C. overnight. To the mixture was added water and the precipitate were collected, washed with water, and dried to afford 47 mg (56%) of desire red product as a yellow solid. LRMS (esi, M−H−) 411.2.

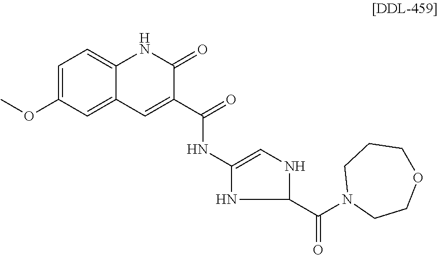 [SMILES: COc1ccc2[nH]c(=O)c(C(=O)NC3=CNC(C(=O)N4CCCOCC4)N3)cc2c1]

(4-nitro-1H-imidazol-2-yl)(1,4-oxazepan-4-yl)methanone (1.1). Step 1: To a mixture of 4-nitro-1H-imidazole-2-carboxylic acid (100 mg, 0.64 mmol), EDC (244 mg, 1.27 mmol), and Hydroxybenzotriazole (122 mg, 0.64 mmol, 80% wt.) in dry DMF (2 mL) was added DIEA (0.33 mL, 1.91 mmol). The mixture was stirred at RT for 10 min followed by the addition of 1,4-oxazepane hydrochloride (105 mg, 0.76 mmol). The mixture was stirred at rt for 2 h and purified directly on a C18 flash column (20 g) eluted with acetonitrile and water (0 to 100%) to afford 76 mg (50%) of compound 1.1 as a yellow solid. (4-amino-1H-imidazol-2-yl)(1,4-oxazepan-4-yl)methanone (1.2). Step 2: To a solution of 1.1 (72 mg, 0.30) in ethanol (5 mL) was added Pd/C (32 mg, 10% wt.). The mixture was charged with H2 balloon, stirred under hydrogen for 1 h, and filtered through a celite plug. The filtrate was concentrated to afford 55 mg of the desired product (87%). N-(2-(1,4-oxazepane-4-carbonyl)-1H-imidazol-4-yl)-6-methoxy-2-oxo-1,2-dihydroquinoline-3-carboxamide (DDL-459). Step-3: To a mixture of 6-methoxy-2-oxo-1,2-dihydroquinoline-3-carboxylic acid (55 mg, 0.25 mmol), EDC (96 mg, 0.50 mmol), and Hydroxybenzotriazole (48 mg, 0.25 mmol, 80% wt.) in dry DMF (2 mL) was added DIEA (0.13 mL, 0.75 mmol). The mixture was stirred at RT for 10 min followed by the addition of (4-amino-1H-imidazol-2-yl)(1,4-oxazepan-4-yl)methanone (52 mg, 0.25 mmol). The mixture was stirred at rt overnight. To the mixture was added water and the precipitate were collected, washed with water, and dried to afford 45 mg (44%) of the desired product as a yellow solid. LRMS (esi, M+H+) 412.4.

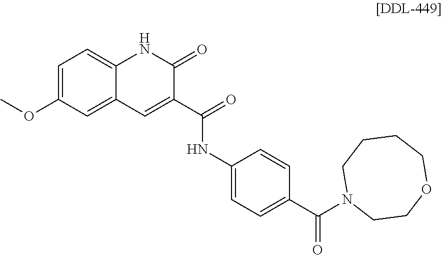 [SMILES: COc1ccc2[nH]c(=O)c(C(=O)Nc3ccc(C(=O)N4CCCCOCC4)cc3)cc2c1]

6-Methoxy-2-oxo-1,2-dihydroquinoline-chromane-3-carboxylic acid (328.8 mg, 1.5 mmol, 1 equiv.) in 70 mL of anhydrous dichloromethane and 17.5 mL of THF was added into a 250 mL round bottom flask. (4-aminophenyl) (1,4-oxazepan-4-yl) methanone (297.36 mg, 1.35 mmol, 0.9 equiv.) was added into the tube followed by the addition of EDAC (345.06 mg, 1.8 mmol, 1.2 equiv.), Hydroxybenzotriazole (222.95) mg, 1.65 mmol, 1.1 equiv.), and N—N, Diisopropylethylamine (0.523 mL, 3 mmol, 2 equiv.) and the resulting reaction mixture were stirred at room temperature overnight. After stirring overnight, the solution changed colors to light green. LC-MS and TLC (Rf: 0.41; DCM: Acetone: Hexanes; 40:20:20) were performed to check the reaction. The reaction mixture was dried and reconstituted using 30 mL of DCM, transferred to a separatory funnel, and extracted with 10% HCL solution with water (2×35 mL). The water layer was extracted and combined organic layers were collected. The organic phase was washed with sodium bicarbonate (2×35 mL), dried with anhydrous Na₂SO₄, filtered, concentrated, and evaporated. The resultant crude compound was purified by using a 4 g silica flash column, eluted with hexanes: ethyl acetate (time/% ethyl acetate: 0/0, 2/0, 13/50, 25/100) then DCM: MeOH (time/% MeOH: 0/0, 3/0, 20/10, 28/15, 30/100) gave desired product along with some starting material. Finally purified using a flash column using DCM: Acetone: Hexanes (40:40:20) and dried to afford the desired product as a pale green solid. The fractions that were corresponding to the interest peaks were dried in a speed vacuum to yield (165.82 mg, pale green solid, 29.1%). ¹H NMR (400 MHz, DMSO-d6) δ 12.57 (s, 1H), 12.40 (s, 1H), 8.92 (s, 1H) 7.76-7.32 (m, 7H), 3.79 (s, 3H), 3.65-3.44 (m, 8H), 1.84-1.71 (m, 2H). LC-MS m/z [M+H]⁺ 422.25.

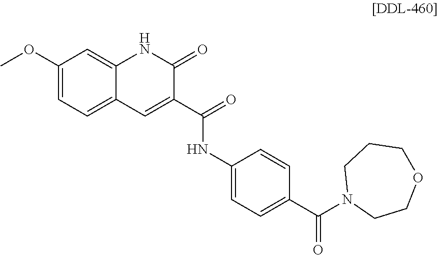 [SMILES: COc1ccc2cc(C(=O)Nc3ccc(C(=O)N4CCCOCC4)cc3)c(=O)[nH]c2c1]

7-methoxy-2-oxo-1,2-dihydroquinoline-3-carboxylic acid (99.517 mg, 0.454 mmol, 1 equiv.) in 75 mL of anhydrous dichloromethane under nitrogen was added into a 250 mL round bottom flask. (4-aminophenyl) (1,4-oxazepan-4-yl) methanone (100.00 mg, 0.454 mmol, 1 equiv.) was added into the tube followed by the addition of EDAC (104.47 mg, 0.545 mmol, 1.2 equiv.), Hydroxybenzotriazole (68.43 mg, 0.499, 1.1 equiv.), and N—N, Diisopropylethylamine (0.158 mL, 0.908 mmol, 2 equiv.), and the resulting reaction mixture were stirred at room temperature for overnight. After stirring overnight, the solution changed colors to cloudy. LC-MS, ad TLC (Rf: 0.76; DCM/20% methanol) were performed to check the reaction. The reaction mixture was transferred to a separatory funnel and extracted with 0.5 M HCL solution with water (3×75 mL). The water layer was extracted and combined organic layers were collected. The organic phase was washed with sodium bicarbonate (3×75 mL), dried with anhydrous Na₂SO₄, filtered, concentrated, and evaporated. The resultant crude compound was purified by using 12 g silica flash column, eluted with hexanes: ethyl acetate (time/% ethyl acetate: 0/0, 2/0, 10/100, 15/100) then DCM: MeOH (time/% MeOH: 0/0, 2/0, 20/10, 30/20). Purification by flash column chromatography afforded DDL 460 (11.2 mg, 5.91%). δ ¹H NMR (400 MHz, DMSO) δ 12.47 (s, 1H), 12.21 (s, 1H), 8.86 (s, 1H), 7.90 (s, 1H), 7.88 (s, 1H), 7.74 (s, 1H), 7.72 (s, 1H), 7.38 (d, J=8.6 Hz, 2H), 6.90 (d, J=4.6 Hz, 1H), 3.83 (s, 3H), 3.65 (s, 4H), 3.57 (s, 2H), 3.44 (s, 2H), 1.84 (s, 1H), 1.71 (s, 1H). LC-MS m/z [M+H]⁺ 422.33.

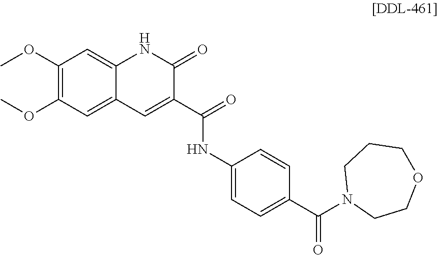 [SMILES: COc1cc2cc(C(=O)Nc3ccc(C(=O)N4CCCOCC4)cc3)c(=O)[nH]c2cc1OC]

7-methoxy-2-oxo-1,2-dihydroquinoline-3-carboxylic acid (113.15 mg, 0.454 mmol, 1 equiv.) in 75 mL of anhydrous dichloromethane under nitrogen was added into a 250 mL round bottom flask. (4-aminophenyl) (1,4-oxazepan-4-yl) methanone (100.00 mg, 0.454 mmol, 1 equiv.) was added into the tube followed by the addition of EDAC (104.47 mg, 0.545 mmol, 1.2 equiv.), Hydroxybenzotriazole (68.43 mg, 0.499, 1.1 equiv.), and N—N, Diisopropylethylamine (0.158 mL, 0.908 mmol, 2 equiv.) and the resulting reaction mixture was stirred at room temperature for overnight. After stirring for overnight, the solution changed colors to cloudy. LC-MS, and TLC (Rf: 0.69; DCM/20% methanol) were performed to check reaction. The reaction mixture was transferred to a separatory funnel and extracted with 0.5 M HCL solution with water (3×75 mL). Water layer was extracted and combined organic layers were collected. The organic phase was washed with sodium bicarbonate (3×75 mL), dried with anhydrous Na₂SO₄, filtered, concentrated, and evaporated. The resultant crude compound was purified by using 10 g silica flash column, eluted with hexanes: ethyl acetate (time/% ethyl acetate: 0/0, 2/0, 10/100, 15/100) then DCM: MeOH (time/% MeOH: 0/0, 2/0, 20/10, 30/20). To get the better purity of DDL 461, the resultant crude compound's fractions were re-purified by using 10 g silica flash column, eluted with hexanes: ethyl acetate (time/% ethyl acetate: 0/0, 2/0, 10/100, 15/100) then DCM: MeOH (time/% MeOH: 0/0, 2/0, 20/10, 30/20). Purification by flash column chromatography afforded DDL 461. The fractions that were corresponding to the interest peaks were dried in speed vacuum to yield (2.23 mg, 1.09%). δ ¹H NMR (400 MHz, DMSO) δ 12.47 (s, 1H), 12.38 (s, 1H), 8.85 (s, 1H), 7.73 (d, J=8.3 Hz, 2H), 7.50 (s, 1H), 7.38 (d, J=8.6 Hz, 2H), 6.95 (s, 1H), 3.85 (s, 3H), 3.79 (s, 3H), 3.61 (d, J=29.9 Hz, 6H), 3.45 (s, 2H), 1.84 (s, 1H), 1.71 (s, 1H). LC-MS m/z [M+H]⁺ 452.25.

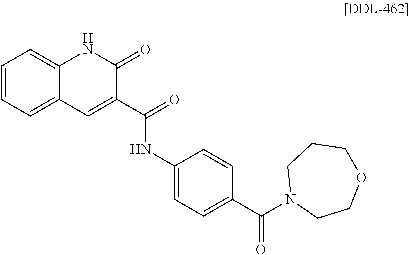 [SMILES: O=C(Nc1ccc(C(=O)N2CCCOCC2)cc1)c1cc2ccccc2[nH]c1=O]

2-oxo-1,2-dihydroquinoline-3-carboxylic acid (50.00 mg, 0.241 mmol, 1 equiv.) in 2 mL of anhydrous dichloromethane under nitrogen was added into a 25 mL of tube. (4-aminophenyl) (1,4-oxazepan-4-yl) methanone (58.22 mg, 0.264 mmol, 1 equiv.) was added into the tube followed by the addition of EDCI (45.02 mg, 0.290 mmol, 1.1 equiv.), Hydroxybenzotriazole (39.77 mg, 0.290 mmol, 1.1 equiv.), and N—N, Diisopropylethylamine (0.0506 mL, 0.58 mmol, 2 equiv.) and the resulting reaction mixture was stirred at room temperature for overnight. After stirring for overnight, the solution changed colors to a milky white. LC-MS, and TLC were performed to check reaction. The reaction mixture was diluted with ethyl acetate (25 mL). Mixture was transferred to a separatory funnel and extracted with 0.5 M HCL solution with water (3×25 mL). Water layer was extracted with ethyl acetate and combined organic layers were collected. The organic phase was washed with sodium bicarbonate (3×25 mL), dried with anhydrous Na₂SO₄, filtered, concentrated, and evaporated. The resultant crude compound was purified by using 12 g silica flash column, eluted with hexanes: ethyl acetate (time/% ethyl acetate: 0/0, 2/0, 10/100, 15/100) then DCM: MeOH (time/% MeOH: 0/0, 2/0, 20/10, 30/20). Purification by flash column chromatography afforded DDL 462. The fractions that were corresponding to the interest peaks were dried in speed vacuum to yield (11.2 mg, 10.38%) as a white powder. ¹H-NMR (400 MHz, DMSO-d₆) δ 12.64 (s, 1H), 12.25 (s, 1H), 8.96 (s, 1H), 7.98 (d, J=9.3 Hz, 1H), 7.75 (d, J=8.3 Hz, 2H), 7.72-7.63 (m, 1H), 7.45 (d, J=8.3 Hz, 1H), 7.39 (d, J=8.6 Hz, 2H), 7.30 (t, J=7.0 Hz, 1H), 3.58 (s, 1H), 3.44 (s, 2H), 3.28 (s, 3H), 2.50 (s, 3H), 1.71 (s, 1H).; LC-MS m/z [M+H]⁺ 392.17.

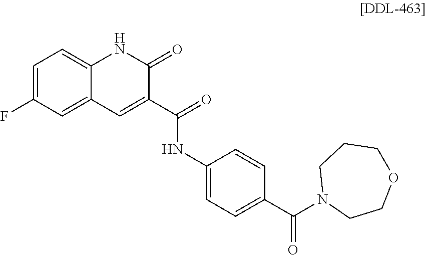 [SMILES: O=C(Nc1ccc(C(=O)N2CCCOCC2)cc1)c1cc2cc(F)ccc2[nH]c1=O]

6-fluoro-2-oxo-1,2-dihydroquinoline-3-carboxylic acid (50.00 mg, 0.241 mmol, 1 equiv.) in 50 mL of anhydrous dichloromethane under nitrogen was added into a 100 mL round bottom flask. (4-aminophenyl) (1,4-oxazepan-4-yl) methanone (53.09 mg, 0.241 mmol, 1 equiv.) was added into the tube followed by the addition of EDAC (55.44 mg, 0.289 mmol, 1.2 equiv.), Hydroxybenzotriazole (36.34 mg, 0.265, 1.1 equiv.), and N—N, Diisopropylethylamine (0.0462 mL, 0.482 mmol, 2 equiv.) and the resulting reaction mixture was stirred at room temperature for overnight. After stirring for overnight, the solution changed colors to a yellowish. LC-MS, and TLC (Rf: 0.64; DCM/20% methanol) were performed to check reaction. The reaction mixture was diluted with ethyl acetate (35 mL). Mixture was transferred to a separatory funnel and extracted with 0.5 M HCL solution with water (3×35 mL). Water layer was extracted with ethyl acetate and combined organic layers were collected. The organic phase was washed with sodium bicarbonate (3×35 mL), dried with anhydrous Na₂SO₄, filtered, concentrated, and evaporated. The resultant crude compound was purified by using 12 g silica flash column, eluted with hexanes: ethyl acetate (time/% ethyl acetate: 0/0, 2/0, 10/100, 15/100) then DCM: MeOH (time/% MeOH: 0/0, 2/0, 20/10, 30/20). Purification by flash column chromatography afforded DDL 463. The fractions that were corresponding to the interest peaks were dried in speed vacuum to yield (3.78 mg, 10.38%) as a brown powder. δ_{H} (400 MHz, DMSO-d₆) 12.73 (1H, s), 12.24 (1H, s), 8.96 (1H, s), 7.89 (1H, d, J 9.0), 7.75 (2H, d, J 8.3), 7.59 (1H, td, J 8.9, 2.9), 7.47 (1H, dd, J 9.1, 4.8), 7.39 (2H, d, J 6.7), 3.51 (4H, d, J 53.5), 3.28 (4H, s), 1.84 (1H, s), 1.71 (1H, s) LC-MS m/z [M+H]⁺ 410.25.

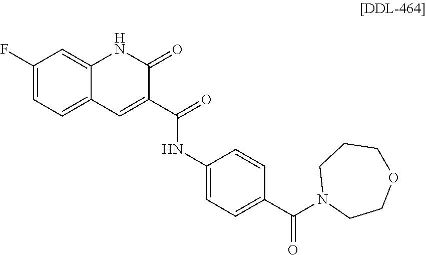 [SMILES: O=C(Nc1ccc(C(=O)N2CCCOCC2)cc1)c1cc2ccc(F)cc2[nH]c1=O]

7-fluoro-2-oxo-1,2-dihydroquinoline-3-carboxylic acid (50.43 mg, 0.241 mmol, 1 equiv.) in 4 mL of anhydrous N, N-Dimethylformamide (DMF) was added into a 25 mL of tube. (4-aminophenyl) (1,4-oxazepan-4-yl) methanone (58.22 mg, 0.241 mmol, 1 equiv.) was added into the tube followed by the addition of EDCI (41.17 mg, 0.265, 1.1 equiv.), Hydroxybenzotriazole (36.34 mg, 0.265, 1.1 equiv.), and N—N, Diisopropylethylamine (0.0462 mL, 0.482 mmol, 2 equiv.) and the resulting reaction mixture was stirred at room temperature for overnight. After stirring for overnight, the starting materials were solubilized. LC-MS, and TLC were performed to check reaction. The reaction mixture was diluted with ethyl acetate (25 mL). Mixture was transferred to a separatory funnel and extracted with sodium bicarbonate (3×25 mL). Water layer was extracted with ethyl acetate and combined organic layers were collected, dried with anhydrous Na₂SO₄, filtered, concentrated, and evaporated. The resultant crude compound was purified by using 12 g silica flash column, eluted with hexanes: ethyl acetate (time/% ethyl acetate: 0/0, 2/0, 10/100, 15/100) then DCM: MeOH (time/% MeOH: 0/0, 2/0, 20/10, 30/20). Purification by flash column chromatography afforded DDL 464. The fractions that were corresponding to the interest peaks were dried in speed vacuum and analyzed by LC-MS. Aniline containing fractions were further purified by HPLC using a C18 SemiPrep column (Time/Acetonitrile %: 0:50, 66/100, 68/100, 70/50, 90/50). Fractions were individually analyzed by LC-MS, and pure fraction (>95%) were pooled and dried in the speed vacuum to yield (3.2 mg, 3.24%) as white powder ¹H NMR (400 MHz, DMSO-d₆) δ 12.70 (s, 1H), 12.09 (s, 1H), 8.97 (s, 1H), 8.09 (dd, J=8.8, 6.1 Hz, 1H), 7.75 (d, J=8.3 Hz, 2H), 7.43-7.35 (m, 2H), 7.25-7.12 (m, 2H), 3.57 (s, 2H), 3.44 (s, 4H), 3.29 (s, 2H), 1.84 (s, 1H), 1.71 (s, 1H).; LC-MS m/z [M+H]⁺ 410.17.

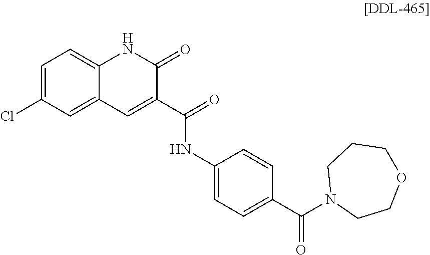 [SMILES: O=C(Nc1ccc(C(=O)N2CCCOCC2)cc1)c1cc2cc(Cl)ccc2[nH]c1=O]

6-chloro-2-oxo-1,2 dihydroquinoline-3-carboxylic acid (49.87 mg, 0.223 mmol, 1 equiv.) in 30 mL of anhydrous dichloromethane, and 10 mL of DMF under nitrogen was added into a 250 mL round bottom flask. (4-aminophenyl) (1,4-oxazepan-4-yl) methanone (39.2 mg, 0.178 mmol, 0.8 equiv.) was added into the tube followed by the addition of EDAC (51.4 mg, 0.268 mmol, 1.2 equiv.), Hydroxybenzotriazole (33.6 mg, 0.245 mmol, 1.1 equiv.), and N—N, Diisopropylethylamine (0.0971 mL, 0.558 mmol, 2.5 equiv.) and the resulting reaction mixture was stirred at room temperature overnight. After stirring overnight, the solution changed colors to slightly yellowish. LC-MS and TLC (Rf: 0.75; 40% DCM: 40% Acetone: 20% Hexanes) were performed to check the reaction. The reaction mixture was transferred to a separatory funnel and extracted with 10% HCL solution with water (3×40 mL). The water layer was extracted and combined organic layers were collected. The organic phase was washed with sodium bicarbonate (3×40 mL), dried with anhydrous Na₂SO₄, filtered, concentrated, and evaporated. The resultant crude compound was purified by using 12 g silica flash column, eluted with hexanes: ethyl acetate (time/% ethyl acetate: 0/0, 2/0, 10/100, 15/100) then 40% DCM: 40% Acetone: 20% Hexanes (time/% DCM: Acetone: Hexanes; 40:40:20): 0/0, 5/50, 10/100, 20/100, 30/100, 40/100, 55/0, 60/50, 70/100). Purification by flash column chromatography afforded DDL 465. The fractions that were corresponding to the interest peaks were dried in speed vacuum to yield (15.2 mg, pale yellow powder, 23.3%). (15.2 mg, pale yellow powder, 23.3%). ¹H NMR (400 MHz, DMSO) δ 12.75 (s, 1H), 12.18 (s, 1H), 8.93 (s, 1H), 8.13 (d, J=4 Hz, 1H), 7.75 (d, J=8.0 Hz, 1H), 7.71 (d, J=4.0 Hz, 1H), 7.69 (d, J=4 Hz, 1H), 7.44 (d, J=8.0 Hz, 1H), 7.40 (s, 1H), 7.38 (s, 1H), 1.71-3.65 (m, 10H). LC-MS m/z [M+H]⁺ 426.17.

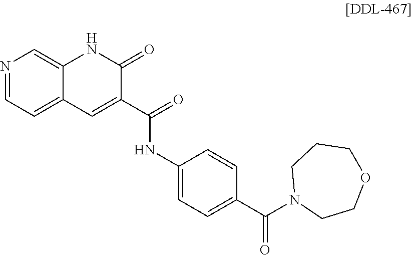 [SMILES: O=C(Nc1ccc(C(=O)N2CCCOCC2)cc1)c1cc2ccncc2[nH]c1=O]

2-oxo-1,2-dihydro-1,7-naphthyridine-3-carboxylic acid (85.01 mg, 0.447 mmol, 1 equiv.) in 40 mL of anhydrous dichloromethane under nitrogen was added into a 100 mL round bottom flask. (4-aminophenyl) (1,4-oxazepan-4-yl) methanone (98.46 mg, 0.447 mmol, 1 equiv.) was added into the tube followed by the addition of 1-Ethyl-3-(3-dimethylaminopropyl)carbodiimide (EDC) (102.83 mg, 0.5364 mmol, 1.2 equiv.), Hydroxybenzotriazole (67.43 mg, 0.4917 mmol, 1.1 equiv.), and N—N, Diisopropylethylamine (155 uL, 0.894 mmol, 2 equiv.), and the resulting reaction mixture was stirred at room temperature overnight. After stirring overnight, the solution changed colors to light yellowish. LC-MS and TLC (Rf: 0.72; 40:40:20: DCM: Hexane: Methanol) were performed to check the reaction. The reaction mixture was transferred to a separatory funnel and extracted with 10% HCL solution with water (3×40 mL). Water layer was extracted and combined organic layers were collected. The organic phase was washed with sodium bicarbonate (3×40 mL), and sodium chloride solution (3×40 mL), dried with anhydrous Na₂SO₄, filtered, concentrated, and evaporated. The resultant crude compound was purified by using a 12 g silica flash column, eluted with hexanes: ethyl acetate (time (min)/% ethyl acetate: 0/0, 5/0, 10/30, 20/60, 25/80, 30/100, and 35/100) then DCM: MeOH (time/% MeOH: 0/0, 2/0, 20/10, 30/20). Purification was reperformed by using a 12 g silica flash column, eluted with hexanes: ethyl acetate (time (min)/% ethyl acetate: 0/0, 4/0, 10/30, 20/55, 25/85, 30/100, and 40/100) then DCM: MeOH (time (min)/% MeOH: 0/0, 5/3, 10/6, 15/9, 20/12, 25/50, 30/70, and 35/100) afforded DDL 467. The fractions that were corresponding to the interest peaks were dried in a speed vacuum to yield (35.01 mg, yellow powder, 19.99%). ¹H NMR (400 MHz, DMSO) δ 12.08 (s, 1H), 8.94 (s, 1H), 8.80 (s, 1H), 8.425 (d, J=4 Hz, 1H), 7.90 (dd, J=8 Hz, 1H), 7.75 (d, J=8 Hz, 2H), 7.40 (d, J=12 Hz, 2H), 3.44-3.70 (m, 8H), 1.84 (s, 1H), 1.71 (s, 1H). LC-MS m/z [M+H]⁺ 393.17.

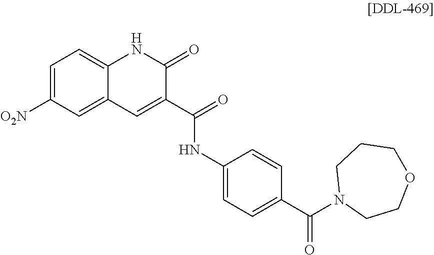 [SMILES: O=C(Nc1ccc(C(=O)N2CCCOCC2)cc1)c1cc2cc([N+](=O)[O-])ccc2[nH]c1=O]

6-chloro-2-oxo-1,2 dihydroquinoline-3-carboxylic acid (53.02 mg, 0.227 mmol, 1 equiv.) in 50 mL of anhydrous dichloromethane under nitrogen was added into a 250 mL round bottom flask. (4-aminophenyl) (1,4-oxazepan-4-yl) methanone (50 mg, 0.227 mmol, 1 equiv.) was added into the tube followed by the addition of EDAC (52.14 mg, 0.272 mmol, 1.2 equiv.), Hydroxybenzotriazole (34.29 mg, 0.25 mmol, 1.1 equiv.), and N—N, Diisopropylethylamine (0.09885 mL, 0.568 mmol, 2.5 equiv.), and the resulting reaction mixture were stirred at room temperature overnight. After stirring overnight, the solution changed colors to slightly yellowish. LC-MS and TLC (Rf: 0.49; 100% Ethyl Acetate) were performed to check the reaction. The reaction mixture was transferred to a separatory funnel and extracted with 10% HCL solution with water (3×50 mL). The water layer was extracted and combined organic layers were collected. The organic phase was washed with sodium bicarbonate (3×50 mL), dried with anhydrous Na₂SO₄, filtered, concentrated, and evaporated. The resultant crude compound was purified by using a 12 g silica flash column, eluted with hexanes: ethyl acetate (time/% ethyl acetate: 0/0, 2/0, 10/100, 15/100) then 40% DCM: 40% Acetone: 20% Hexanes (time/% DCM: Acetone: Hexanes; 40:40:20): 0/0, 5/50, 10/100, 20/100, 30/100, 40/100, 55/0, 60/50, 70/100). Purification by flash column chromatography afforded DDL 469. The fractions that were corresponding to the interest peaks were dried in a speed vacuum to yield (20.19 mg, light green powder, 20.19%). ¹H NMR (400 MHz, DMSO) δ 13.07 (s, 1H), 11.91 (s, 1H), 9.13 (s, 1H), 9.02 (s, 1H), 8.445 (d, J=4 Hz, 1H), 8.42 (d, 1H), 7.76 (d, J=8.0 Hz, 2H), 7.56 (d, J=8.0 Hz, 1H), 7.40 (d, J=8.0 Hz, 1H), 1.71-3.65 (m, 10H). LC-MS m/z [M+H]⁺ 437.17.

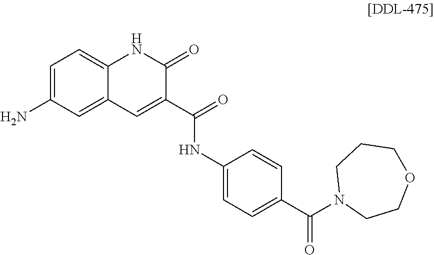 [SMILES: Nc1ccc2[nH]c(=O)c(C(=O)Nc3ccc(C(=O)N4CCCOCC4)cc3)cc2c1]

To a solution of DDL 469 (16.8 mg, 0.0385 mmol, 1 Equiv.) in ethanol (6 mL) was added Pd/C (5.3 mg, 10% wt.). The mixture was charged with an H2 balloon, stirred under hydrogen for 16 hours, and filtered through a celite plug. The filtrate was concentrated to afford DDL475 (10.5 mg, Dark yellow powder 67.13%). ¹H NMR (400 MHz, d6-DMSO) δ 12.55 (s, 1H), 8.69 (s, 1H), 7.75-6.48 (m, 8H), 5.40 (s, 1H), 5.23 (s, 1H), 3.64-3.45 (m, 8H), 2.03-1.80 (m, 2H). LC-MS m/z [M+H]⁺ 407.25.

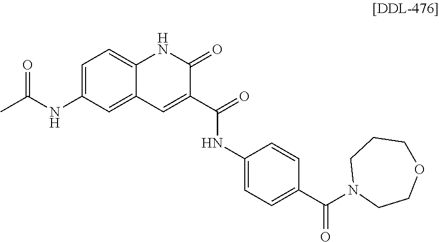 [SMILES: CC(=O)Nc1ccc2[nH]c(=O)c(C(=O)Nc3ccc(C(=O)N4CCCOCC4)cc3)cc2c1]

To a solution of the DDL 475 (0.0312 mmol) and Triethylamine (0.125 mmol) was added Acetyl chloride (0.1872 mmol) in DCM (3 mL) under a nitrogen atmosphere while keeping the temperature below 0 degrees. The mixture was warmed to rt and stirred for 16 h. Once the reaction was complete as indicated by TLC, 1M HCl (2×, 5 mL) was added, sodium bicarbonate (2×, 5 mL), brine (2×, 5 mL), was extracted with DCM, and the organic layer was separated, and concentrated under a vacuum to give the desired product. The resultant crude compound was purified by using a 4 g silica flash column, eluted with DCM: MeOH (time/% MeOH: 0/0, 5/0, 20/10, and 40/100). Purification by flash column chromatography afforded 476 (5.8 mg, light green powder 41.4%). ¹H NMR (400 MHz, d6-DMSO) δ 12.35 (s, 1H), 10.14 (s, 1H), 8.84 (s, 1H), 8.14-7.37 (m, 7H), 4.05 (s, 1H), 3.65-3.44 (m, 8H), 2.04 (s, 3H), 1.83-1.71 (m, 2H). LC-MS m/z [M+H]⁺ 449.25.

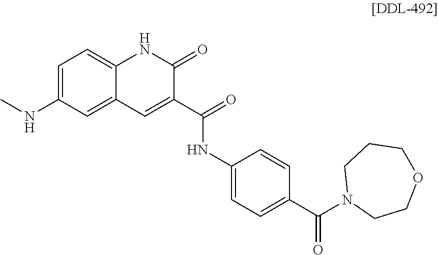 [SMILES: CNc1ccc2[nH]c(=O)c(C(=O)Nc3ccc(C(=O)N4CCCOCC4)cc3)cc2c1]

DDL475 (8.76 mg, 0.02156 mmol, 1 Equiv.) and methyl methane sulfonate (2.5 Equiv.) were added to a stirred solution of sodium bicarbonate (5 Equiv.) and celite (21.56 mg; 1 mmol/1 g) in ethanol (3 mL). The reaction mixture was refluxed for 2 h under nitrogen. After checking the TLC, the reaction mixture was cooled to room temperature and then the celite was removed through filtration (via sintered glass funnel), and the filtrate was collected, and dried in a speed vacuum. The crude compound was purified by using a 4 g silica flash column, eluted with DCM: MeOH (time/% Ethyl acetate: 5/0, 20/15, 30/100). The fraction that was corresponding to the interest peak was dried in a speed vacuum to yield DDL492 (4.5 mg, 49.66%). ¹H NMR (400 MHz, CDCl₃) δ 12.51 (s, 1H), 8.70 (s, 1H), 7.77-7.00 (m, 8H), 3.70 (s, 3H), 3.65-3.45 (m, 8H), 1.84-1.71 (m, 2H). LC-MS m/z [M+H]⁺ 421.25

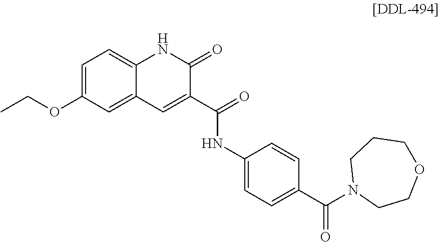 [SMILES: CCOc1ccc2[nH]c(=O)c(C(=O)Nc3ccc(C(=O)N4CCCOCC4)cc3)cc2c1]

An 8 mL flask equipped with a magnetic stir bar was charged with aldehydes (1.0 mmol), 1-Hydroxycyclohexyl phenyl ketone (204.3 mg, 1.0 mmol), sodium hydroxide (80.0 mg, 2.0 mmol), and DME (1.5 mL). The flask was then sealed with a cap, and the reaction mixture was allowed to stir at 80 degrees. The reaction progress was monitored by TLC until no starting material was observed. The cooled reaction mixture was diluted with water. The resulting mixture was then extracted with diethyl ether to remove 1-HCPK and DME before acidification. The aqueous layer was then neutralized by 12N HCl, and extracted with ethyl acetate. The residue was purified by recrystallization to afford 2-chloro-6-(ethyl oxy) quinoline-3-carboxylic acid (101 mg, 40.1%, brown powder). Suspension of corresponding 2-chloro-6-(ethyl oxy) quinoline-3-carboxylic acid (95.5 mg, 0.38 mmol) in glacial acetic acid (5 mL) and water (1 mL) was boiled with stirring for 20 h. The reaction was monitored by TLC. After cooling, water (30 mL) was added and the solid product was filtered, washed with water, and then dried in a vacuum oven at 60° C. to afford 6-ethyl-oxy-2-oxo-1,2-dihydroquinoline-3-carboxylic acid (47.3 mg, 53.4%, brown powder). 6-ethyl-oxy-2-oxo-1,2-dihydroquinoline-3-carboxylic acid (30 mg, 0.129 mmol, 1 Equiv.), HOBt (19.34 mg, 0.141 mmol, 1.1 Equiv.), and EDC (29.5 mg, 0.154 mmol, 1.2 Equiv.) were added into the round bottom flask with a stir bar. 10 mL of anhydrous DCM and 2 eq. of DIPEA were added to the round bottom flask, and (4-aminophenyl) (1,4-oxazepan-4-yl)-methanone (1 Equiv.) was then added to the round bottom flask and stir for overnight. After being stirred overnight, the solution changed colors to yellowish. Atmospheric pressure chemical ionization was performed to check the reaction. The reaction mixture was reconstituted using DCM and transferred to a separatory funnel, the water layer was extracted and combined organic layers were collected. The organic phase was washed with sodium bicarbonate (3×10 mL), and brine solution (20 mL), dried with anhydrous Na₂SO₄, filtered, concentrated, and evaporated. The resultant crude compound was purified by using a 4 g silica flash column, eluted with Hexane: Ethyl acetate (time/% Ethyl acetate: 0/0, 5/0, 25/100), and eluted with DCM: MeOH (time/% MeOH: 0/0, 5/0, 15/10, 25/20, 40/100) afforded DDL 494. The fractions that corresponded to the interest peaks were dried in a speed vacuum to yield DDL494 (19.2 mg, 34.2%, yellow powder). ¹H NMR (400 MHz, CDCl₃) δ 12.11 (s, 1H), 11.49 (s, 1H), 8.99 (s, 1H) 7.81-7.15 (m, 7H), 3.87-3.68 (m, 8H), 4.09 (q, J=8 Hz, 2H), 2.06 (m, 2H), 1.46 (t, J=4 Hz, 3H). LC-MS m/z [M+H]⁺ 436.17

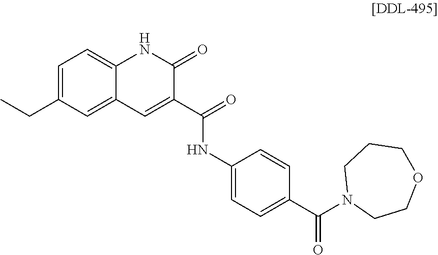 [SMILES: CCc1ccc2[nH]c(=O)c(C(=O)Nc3ccc(C(=O)N4CCCOCC4)cc3)cc2c1]

Suspension of corresponding 2-chloro-6-(ethyl) quinoline-3-carboxylic acid (0.2 mmol) in glacial acetic acid (5 mL) and water (1 mL) was boiled with stirring for 20 h. The reaction was monitored by TLC. After cooling, water (30 mL) was added and the solid product was filtered, washed with water, and then dried in a vacuum oven at 60° C. to yield 6-ethyl-2-oxo-1,2-dihydroquinoline-3-carboxylic acid (25 mg, 57.5%). LC-MS m/z [M+H]⁺ 218.00. 6-ethyl-2-oxo-1,2-dihydroquinoline-3-carboxylic acid (25 mg, 0.115 mmol, 1 Equiv.), HOBt (17.16 mg, 0.0127 mmol, 1.1 Equiv.), and EDC (26.45 mg, 0.138 mmol, 1.2 Equiv.) were added into the round bottom flask with a stir bar. 5 mL of anhydrous DCM and 2 eq. of DIPEA were added to the round bottom flask, and (4-aminophenyl) (1,4-oxazepan-4-yl)-methanone (1 Equiv.) was then added to the round bottom flask and stir overnight. After being stirred overnight, the solution changed colors to yellowish. Atmospheric pressure chemical ionization was performed to check the reaction. The reaction mixture was reconstituted using DCM and transferred to a separatory funnel, the water layer was extracted and combined organic layers were collected. The organic phase was washed with sodium bicarbonate (2×5 mL), and brine solution (10 mL), dried with anhydrous Na₂SO₄, filtered, concentrated, and evaporated. The resultant crude compound was purified by using a 4 g silica flash column, eluted with Hexane: Ethyl acetate (time/% Ethyl acetate: 0/0, 5/0, 25/100), and eluted with DCM: MeOH (time/% MeOH: 0/0, 5/0, 15/10, 25/20, 40/100) afforded DDL 495. The fractions that corresponded to the interest peaks were dried in a speed vacuum to yield DDL495 (12.53 mg, 26%, light yellow powder)¹H NMR (400 MHz, CDCl₃) δ 12.05 (s, 1H), 10.61 (s, 1H), 9.05 (s, 1H) 7.85-7.28 (m, 7H), 3.87-3.60 (m, 8H), 2.77 (q, J=8 Hz, 2H), 2.06-1.87 (m, 2H), 1.31 (t, J=8 Hz, 3H). LC-MS m/z [M+H]⁺ 420.25.

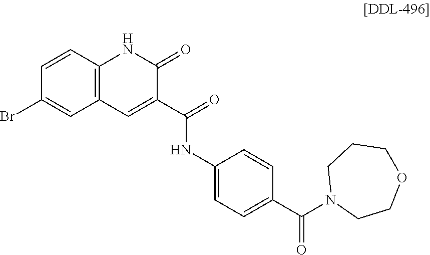 [SMILES: O=C(Nc1ccc(C(=O)N2CCCOCC2)cc1)c1cc2cc(Br)ccc2[nH]c1=O]

6-Bromo-2-oxo-1,2-dihydroquinoline-3-carboxylic acid (50 mg, 0.187 mmol, 1 Equiv.), HOBt (28.2 mg, 0.2057 mmol, 1.1 Equiv.), and EDC (42.75 mg, 0.223 mmol, 1.2 Equiv.) were added into the round bottom flask with a stir bar. 3 mL of anhydrous DMF, 7 mL of DCM, and 2 eq. of DIPEA were added to the round bottom flask, and (4-aminophenyl) (1,4-oxazepan-4-yl)-methanone (1 Equiv.) was then added to the round bottom flask and stir for overnight. After being stirred overnight, the solution changed colors to yellowish. Atmospheric pressure chemical ionization was performed to check the reaction. The reaction mixture was reconstituted using 10 mL of DCM and transferred to a separatory funnel, the water layer was extracted and combined organic layers were collected. The organic phase was washed with sodium bicarbonate (3×10 mL), and brine solution (20 mL), dried with anhydrous Na₂SO₄, filtered, concentrated, and evaporated. The resultant crude compound was purified by using a 4 g silica flash column, eluted with Hexane: Ethyl acetate (time/% Ethyl acetate: 0/0, 5/0, 25/100), and eluted with DCM: MeOH (time/% MeOH: 0/0, 5/0, 15/10, 25/20, 40/100), and the fractions that corresponded to the interest peaks were dried in a speed vacuum to yield DDL 496, (25.8 mg, 29.28%, White powder). ¹H NMR (400 MHz, DMSO-d6) δ 12.74 (s, 1H), 12.14 (s, 1H), 8.27 (s, 1H) 7.82-7.37 (m, 8H), 3.65-3.44 (m, 8H), 1.84-1.71 (m, 2H), LC-MS m/z [M]⁺ 470.17.

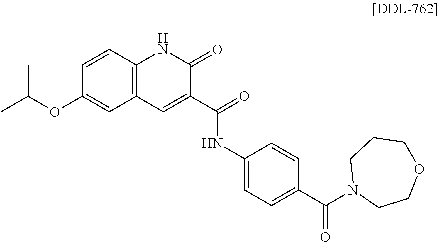 [SMILES: CC(C)Oc1ccc2[nH]c(=O)c(C(=O)Nc3ccc(C(=O)N4CCCOCC4)cc3)cc2c1]

6-isopropoxy-2-oxo-1,2-dihydroquinoline-3-carboxylic acid (30 mg, 0.121 mmol, 1 Equiv.), HOBt (18.24 mg, 0.133 mmol, 1.1 Equiv.), and EDC (27.99 mg, 0.146 mmol, 1.2 Equiv.) were added into the round bottom flask with a stir bar. 10 mL of anhydrous DCM and 2 eq. of DIPEA were added to the round bottom flask, and (4-aminophenyl) (1,4-oxazepan-4-yl)-methanone (26.65 mg, 0.121 mmol, 1 Equiv.) was then added to the round bottom flask and stirred overnight. After being stirred overnight, the solution changed colors to yellowish. Atmospheric pressure chemical ionization was performed to check the reaction. The reaction mixture was reconstituted using DCM and transferred to a separatory funnel, the water layer was extracted and combined organic layers were collected. The organic phase was washed with sodium bicarbonate (3×10 mL), and brine solution (20 mL), dried with anhydrous Na₂SO₄, filtered, concentrated, and evaporated. The resultant crude compound was purified by using a 4 g silica flash column, eluted with Hexane: Ethyl acetate (time/% Ethyl acetate: 0/0, 5/0, 25/100), and eluted with DCM: MeOH (time/% MeOH: 0/0, 5/0, 15/10, 25/20, 40/100) afforded DDL 762. The fractions that corresponded to the interest peaks were dried in a speed vacuum to yield DDL762 (12.4 mg, 23%, light-yellow powder). ¹H NMR (400 MHz, CDCl₃) δ 12.09 (s, 1H), 10.36 (s, 1H), 9.00 (s, 1H) 7.84-6.64 (m, 7H), 4.62 (q, J=4 Hz, 1H), 3.83-3.59 (m, 8H), 2.06-1.86 (m, 2H), 1.39 (d, J=4 Hz, 6H). LC-MS m/z [M+H]⁺ 450.50.

### Reaction Scheme by Batch Chemistry Synthesis (Coumarin Series)

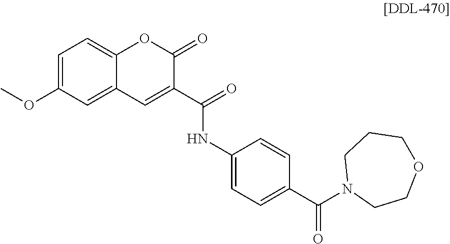 [SMILES: COc1ccc2oc(=O)c(C(=O)Nc3ccc(C(=O)N4CCCOCC4)cc3)cc2c1]

6-methoxy-2-oxo-2H-chromene-3-carboxylic acid (50.00 mg, 0.227 mmol, 1 equiv.) in 30 mL of anhydrous dichloromethane under nitrogen was added into a 100 mL round bottom flask. (4-aminophenyl) (1,4-oxazepan-4-yl) methanone (50.00 mg, 0.227 mmol, 1 equiv.) was added into the tube followed by the addition of EDAC (52.14 mg, 0.272 mmol, 1.2 equiv.), Hydroxybenzotriazole (34.24 mg, 0.2497 mmol, 1.1 equiv.), and N—N, Diisopropylethylamine (79 μL, 0.454 mmol, 2 equiv.), and the resulting reaction mixture were stirred at room temperature overnight. After stirring overnight, the solution changed colors to light yellowish. LC-MS and TLC (Rf: 0.34; 100% ethyl acetate) were performed to check the reaction. The reaction mixture was transferred to a separatory funnel and extracted with 10% HCL solution with water (3×30 mL). The water layer was extracted and combined organic layers were collected. The organic phase was washed with sodium bicarbonate (3×30 mL), and sodium chloride solution (3×30 mL), dried with anhydrous Na₂SO₄, filtered, concentrated, and evaporated. The resultant crude compound was purified by using a 12 g silica flash column, eluted with hexanes: ethyl acetate (time (min)/% ethyl acetate: 0/0, 5/0, 10/30, 20/60, 25/80, 30/100, and 35/100) then DCM: MeOH (time/% MeOH: 0/0, 2/0, 20/10, 30/20). Re-purification by using a 12 g silica flash column, eluted with hexanes: ethyl acetate (time (min)/% ethyl acetate: 0/0, 4/0, 10/30, 20/55, 25/85, 30/100, and 40/100) then DCM: MeOH (time (min)/% MeOH: 0/0, 15/10, 20/15, 25/20, 30/80, 35/100) afforded DDL 470. The fractions that were corresponding to the interest peaks were dried in a speed vacuum to yield (28.64 mg, light yellow powder, 29.81%). ¹H NMR (400 MHz, DMSO) δ 10.78 (s, 1H), 8.85 (s, 1H), 7.75 (d, J=8 Hz, 2H), 7.555 (d, J=4.0 Hz, 1H), 7.47 (d, J=8 Hz, 1H), 7.39 (d, J=8 Hz, 1H), 7.35 (d, 1H), 7.325 (d, J=4.0 Hz, 1H), 3.80 (s, 3H), 3.70 (s, 1H), 3.65 (s, 4H), 3.57 (s, 1H), 3.43 (s, 2H), 1.84 (s, 1H), 1.70 (s, 1H). LC-MS m/z [M+H]⁺ 423.17.

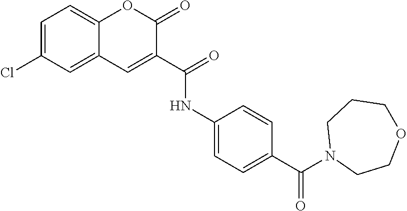 [SMILES: O=C(Nc1ccc(C(=O)N2CCCOCC2)cc1)c1cc2cc(Cl)ccc2oc1=O]

6-chloro-2-oxo-2H-chromene-3-carboxylic acid (101.75 mg, 0.453 mmol, 1 equiv.) in 50 mL of anhydrous dichloromethane under nitrogen was added into a 250 mL round bottom flask. (4-aminophenyl) (1,4-oxazepan-4-yl) methanone (100.00 mg, 0.453 mmol, 1 equiv.) was added into the tube followed by the addition of EDAC (104.21 mg, 0.5436 mmol, 1.2 equiv.), Hydroxybenzotriazole (68.34 mg, 0.4983 mmol, 1.1 equiv.), and N—N, Diisopropylethylamine (0.158 mL, 0.906 mmol, 2 equiv.) and the resulting reaction mixture was stirred at room temperature overnight. After stirring overnight, the solution changed colors to yellowish. LC-MS and TLC (Rf: 0.29; 100% ethyl acetate) were performed to check the reaction. The reaction mixture was transferred to a separatory funnel and extracted with 10% HCL solution with water (3×50 mL). The water layer was extracted and combined organic layers were collected. The organic phase was washed with sodium bicarbonate (3×50 mL), and sodium chloride solution (3×50 mL), dried with anhydrous Na₂SO₄, filtered, concentrated, and evaporated. The resultant crude compound was purified by using 12 g silica flash column, eluted with hexanes: ethyl acetate (time/% ethyl acetate: 0/0, 2/0, 10/100, 15/100) then DCM: MeOH (time/% MeOH: 0/0, 2/0, 20/10, 30/20). Purification by flash column chromatography afforded DDL 471. The fractions that were corresponding to the interest peaks were dried in speed vacuum to yield (51.5 mg, yellow powder, 26.69%). ¹H NMR (400 MHz, DMSO) δ 10.69 (s, 1H), 8.82 (s, 1H), 8.12 (d, J=4 Hz, 1H), 7.79 (d, 1H), 7.765 (d, J=4 Hz, 1H), 7.75, (s, 1H), 7.74 (d, J=8 Hz, 1H), 7.57 (d, J=8 Hz, 1H), 7.39 (d, J=8 Hz, 1H), 3.70 (s, 2H), 3.65 (s, 3H), 3.57 (s, 1H), 3.43 (s, 2H), 1.84 (s, 1H), 1.70 (s, 1H). LC-MS m/z [M+H]⁺ 427.25.

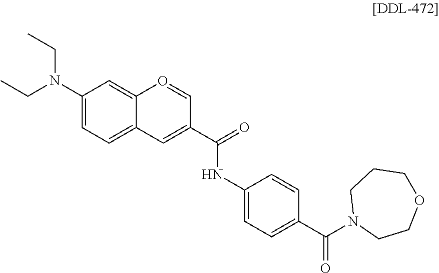

7-(diethylamino)-2-oxo-2H-chromene-3-carboxylic acid (118.36 mg, 0.453 mmol, 1 equiv.) in 45 mL of anhydrous dichloromethane under nitrogen was added into a 250 mL round bottom flask. (4-aminophenyl) (1,4-oxazepan-4-yl) methanone (100.00 mg, 0.453 mmol, 1 equiv.) was added into the tube followed by the addition of EDAC (104.21 mg, 0.5436 mmol, 1.2 equiv.), Hydroxybenzotriazole (68.34 mg, 0.4983 mmol, 1.1 equiv.), and N—N, Diisopropylethylamine (0.158 mL, 0.906 mmol, 2 equiv.) and the resulting reaction mixture was stirred at room temperature overnight. After stirring overnight, the solution changed colors to bright yellowish. LC-MS and TLC (Rf: 0.39; 30% hexane:70% ethyl acetate) were performed to check the reaction. The reaction mixture was transferred to a separatory funnel and extracted with 10% HCL solution with water (3×45 mL). The water layer was extracted and combined organic layers were collected. The organic phase was washed with sodium bicarbonate (3×45 mL), and sodium chloride solution (3×45 mL), dried with anhydrous Na₂SO₄, filtered, concentrated, and evaporated. The resultant crude compound was purified by using a 12 g silica flash column, eluted with hexanes: ethyl acetate (time/% ethyl acetate: 0/0, 2/0, 10/100, 15/100) then DCM: MeOH (time/% MeOH: 0/0, 2/0, 20/10, 30/20). Purification by flash column chromatography afforded DDL 472. The fractions that were corresponding to the interest peaks were dried in a speed vacuum to yield (116.32 mg, yellow powder, 55.42%). ¹H NMR (400 MHz, DMSO) δ 10.81 (s, 1H), 8.74 (s, J=0.6 Hz, 1H), 7.73 (d, J=8 Hz, 2H), 7.36 (d, J=8 Hz, 2H), 6.83 (d, J=4 Hz, 1H), 6.80 (d, J=4 Hz, 1H), 6.64 (d, 1H), 3.65 (m, 8H), 3.47 (q, J=8.0 Hz, 4H), 1.83 (s, 1H), 1.70 (s, 1H), 1.11 (t, J=6.0 Hz, 6H). LC-MS m/z [M+H]⁺ 464.43.

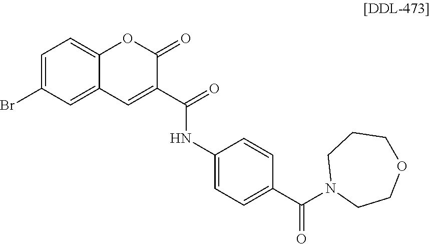 [SMILES: O=C(Nc1ccc(C(=O)N2CCCOCC2)cc1)c1cc2cc(Br)ccc2oc1=O]

6-bromo-2-oxo-2H-chromene-3-carboxylic acid (61.07 mg, 0.227 mmol, 1 equiv.) in 20 mL of anhydrous dichloromethane and 5 mL tetrahydrofuran was added into a 100 mL round bottom flask under nitrogen gas. (4-aminophenyl) (1,4-oxazepan-4-yl) methanone (50 mg, 0.227 mmol, 1 equiv.) was added into the tube followed by the addition of 1-Ethyl-3-(3-dimethylaminopropyl)carbodiimide (EDC) (52.14 mg, 0.272 mmol, 1.2 equiv.), Hydroxybenzotriazole (37.30 mg, 0.272 mmol, 1.1 equiv.), and N—N, Diisopropylethylamine (94.3 uL, 0.544 mmol, 2 equiv.), and the resulting reaction mixture was stirred at room temperature overnight. After stirring overnight, the solution changed colors to light yellowish. LC-MS and TLC (Rf: 0.29; 100% ethyl acetate) were performed to check the reaction. The reaction mixture was transferred to a separatory funnel and extracted with 10% HCL solution with water (3×30 mL). The water layer was extracted and combined organic layers were collected. The organic phase was washed with sodium bicarbonate (3×30 mL) and dried with anhydrous Na₂SO₄, filtered, concentrated, and evaporated. The resultant crude compound was purified by using 12 g silica flash column, eluted with hexanes: ethyl acetate (time (min)/% ethyl acetate: 0/0, 5/0, 30/100, and 35/100). The fractions that were corresponding to the interest peaks were dried in a speed vacuum to yield (54.37 mg, white powder, 49.88%). 1H NMR (400 MHz, DMSO) δ 10.69 (s, 1H), 8.81 (s, 1H), 8.24 (d, J=2.4 Hz, 1H), 7.89 (dd, J=8.8, 2.4 Hz, 1H), 7.74 (d, J=8.2 Hz, 2H), 7.50 (d, J=8.9 Hz, 1H), 7.39 (d, J=8.6 Hz, 2H), 3.69 (s, 2H), 3.65 (s, 3H), 3.57 (s, 1H), 3.43 (s, 2H), 1.84 (s, 1H), 1.70 (s, 1H).

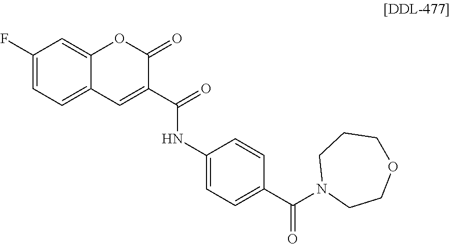 [SMILES: O=C(Nc1ccc(C(=O)N2CCCOCC2)cc1)c1cc2ccc(F)cc2oc1=O]

6-chloro-2-oxo-1,2 dihydroquinoline-3-carboxylic acid (30.00 mg, 0.143 mmol, 1 equiv.) in 30 mL of anhydrous dichloromethane under nitrogen was added into a 250 mL round bottom flask. (4-aminophenyl) (1,4-oxazepan-4-yl) methanone (31.4 mg, 0.143 mmol, 1 equiv.) was added into the tube followed by the addition of EDAC (33.16 mg, 0.173 mmol, 1.2 equiv.), Hydroxybenzotriazole (21.57 mg, 0.157 mmol, 1.1 equiv.), and N—N, Diisopropylethylamine (0.05 mL, 0.288 mmol, 2.5 equiv.) and the resulting reaction mixture was stirred at room temperature overnight. After stirring overnight, the solution changed colors to pale green. LC-MS and TLC (Rf: 0.80; 100% Ethyl Acetate) were performed to check the reaction. The reaction mixture was transferred to a separatory funnel and extracted with 10% HCL solution with water (3×40 mL). The water layer was extracted and combined organic layers were collected. The organic phase was washed with sodium bicarbonate (3×40 mL), dried with anhydrous Na₂SO₄, filtered, concentrated, and evaporated. The resultant crude compound was purified by using 12 g silica flash column, eluted with hexanes: ethyl acetate (time/% ethyl acetate: 0/0, 2/0, 10/100, 15/100) then DCM: MeOH (time/% MeOH: 0/0, 2/0, 20/10, 30/20). Purification by flash column chromatography afforded DDL 477. The fractions that were corresponding to the interest peaks were dried in a speed vacuum to yield (4.6 mg, pale green powder, 7.8%), ¹H NMR (400 MHz, DMSO) δ 8.97 (s, 1H), 8.09 (d, J=4 Hz, 1H), 7.75 (d, J=12.0 Hz, 2H), 7.39 (d, J=8.0 Hz, 2H), 7.25-7.10 (m, 2H), 1.71-3.65 (m, 10H). LC-MS m/z [M+H]⁺ 411.17.

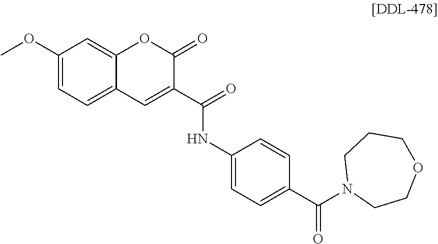 [SMILES: COc1ccc2cc(C(=O)Nc3ccc(C(=O)N4CCCOCC4)cc3)c(=O)oc2c1]

7-methoxy-2-oxo-3-carboxylic acid (50 mg, 0.227 mmol, 1 equiv.) in 50 mL of anhydrous dichloromethane under nitrogen was added into a 250 mL round bottom flask. (4-aminophenyl) (1,4-oxazepan-4-yl) methanone (50 mg, 0.227 mmol, 1 equiv.) was added into the tube followed by the addition of EDAC (52.3 mg, 0.273. mmol, 1.2 equiv.), Hydroxybenzotriazole (34.29 mg, 0.250 mmol, 1.1 equiv.), and N—N, Diisopropylethylamine (79 μL, 0.454 mmol, 2 equiv.), and the resulting reaction mixture was stirred at room temperature overnight. After stirring overnight, the solution changed colors to slightly yellowish. LC-MS and TLC (Rf: 0.80; 40% DCM: 40% Acetone: 20% Hexanes) were performed to check the reaction. The reaction mixture was transferred to a separatory funnel and extracted with 10% HCL solution with water (3×50 mL). The water layer was extracted and combined organic layers were collected. The organic phase was washed with sodium bicarbonate (3×50 mL), dried with anhydrous Na₂SO₄, filtered, concentrated, and evaporated. The resultant crude compound was purified by using 12 g silica flash column, eluted with hexanes: ethyl acetate (time/% ethyl acetate: 0/0, 2/0, 20/50, 40/100) then DCM: MeOH (time/% MeOH: 0/0, 2/0, 20/10, 30/50, 40/100). Purification by flash column chromatography afforded DDL 478. The fractions that were corresponding to the interest peaks were dried in speed vacuum to yield (22.7 mg, light green powder, 23.67%). ¹H NMR (400 MHz, CDCl₃) δ 10.92 (s, 1H), 8.94 (s, 1H), 7.78 (d, J=8.0 Hz, 2H), 7.63 (d, J=8.0 Hz, 1H), 7.43 (d, J=8.0 Hz, 2H), 6.97 (dd, J=8.7, 2.4 Hz, 1H), 6.90 (d, 1H), 3.93 (s, 3H), 3.89-3.43 (m, 8H), 2.11-1.24 (m, 2H) LC-MS m/z [M+H]⁺ 423.17.

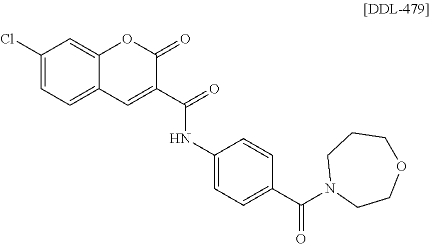 [SMILES: O=C(Nc1ccc(C(=O)N2CCCOCC2)cc1)c1cc2ccc(Cl)cc2oc1=O]

7-chloro-2-oxo-2H-Chromene-3-carboxylic acid (100.00 mg, 0.446 mmol, 1 Equiv.) in 40 mL of anhydrous dichloromethane under nitrogen was added into a 250 mL round bottom flask. (4-aminophenyl) (1,4-oxazepan-4-yl) methanone (98.24 mg, 0.446 mmol, 1 equiv.) was added into the tube followed by the addition of 1-Ethyl-3-(3-dimethylaminopropyl)carbodiimide (EDC) (102.6 mg, 0.5352 mmol, 1.2 equiv.), Hydroxybenzotriazole (66.3 mg, 0.4906 mmol, 1.1 equiv.), and N—N, Diisopropylethylamine (194.2 μL, 1.115 mmol, 2.5 equiv.), and the resulting reaction mixture were stirred at room temperature overnight. After stirring overnight, the solution changed colors to brownish. LC-MS and TLC (Rf: 0.58; 100% DCM) were performed to check the reaction. The reaction mixture was transferred to a separatory funnel and extracted with 100% HCL solution with water (3×40 mL). The water layer was extracted and combined organic layers were collected. The organic phase was washed with sodium bicarbonate (3×40 mL), dried with anhydrous Na₂SO₄, filtered, concentrated, and evaporated. The resultant crude compound was purified by using a 12 g silica flash column, eluted with hexanes: ethyl acetate (time/% ethyl acetate: 0/0, 2/0, 20/50, 40/100) then DCM: MeOH (time/% MeOH: 0/0, 2/0, 20/10, 30/50, 40/100). Purification by ash column chromatography afforded DDL 479. The fractions that were corresponding to the interest peaks were dried in a speed vacuum to yield (67.64 mg, light green powder, 35.6%). ¹H NMR (400 MHz, CDCl₃) δ 10.84 (s, 1H), 8.98 (s, 1H), 7.78-7.40 (m, 7H), 3.85-3.56 (m, 8H), 2.05-1.85 (m, 2H). LC-MS m/z [M+H]⁺ 427.17.

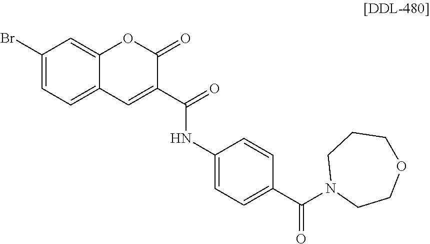 [SMILES: O=C(Nc1ccc(C(=O)N2CCCOCC2)cc1)c1cc2ccc(Br)cc2oc1=O]

7-bromo-2-oxo-2H-chromane-3-carboxylic acid (50 mg, 0.186 mmol, 1 Equiv.) in 30 mL of THF and 10 mL of DMF under nitrogen was added into a 100 mL round bottom flask. (4-aminophenyl) (1,4-oxazepan-4-yl) methanone (40.94 mg, 0.186 mmol, 1 equiv.) was added into the tube followed by the addition of EDC (71.2 mg, 0.372 mmol, 2 equiv.), Hydroxybenzotriazole (27.64 mg, 0.205 mmol, 1.1 equiv.), and N—N, Diisopropylethylamine (97.1 μL, 0.558 mmol, 3 equiv.), and the resulting reaction mixture were stirred at room temperature overnight. After stirring for 72 hours, the solution changed colors to yellowish. LC-MS and TLC (Rf: 0.65; 85% DCM: 15% MeOH) were performed to check the reaction. The reaction mixture was dried and reconstituted using 30 mL of DCM. Shortly after, the crude reaction mixture was transferred to a separatory funnel and extracted with 10% HCL solution with water (3×30 mL). The water layer was extracted and combined organic layers were collected. The organic phase was washed with sodium bicarbonate (3×30 mL), dried with anhydrous Na₂SO₄, filtered, concentrated, and evaporated. The resultant crude compound was purified by using a 4 g silica flash column, eluted with hexanes: ethyl acetate (time/% ethyl acetate: 0/0, 2/0, 20/50, 40/100) then DCM: MeOH (time/% MeOH: 0/0, 5/0, 25/10, 30/100). Purification by flash column chromatography afforded DDL 480. The fractions that corresponded to the interest peaks were dried in a speed vacuum to yield (15.82 mg, of a yellow powder, 18.06%). ¹H NMR (400 MHz, CDCl₃) δ 10.84 (s, 1H), 8.97 (s, 1H), 7.79-7.43 (m, 7H), 3.85-3.56 (m, 8H), 2.05 (m, 2H). LC-MS m/z [M+2H]⁺ 473.42.

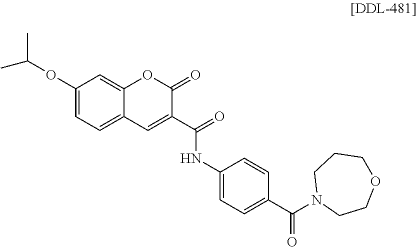 [SMILES: CC(C)Oc1ccc2cc(C(=O)Nc3ccc(C(=O)N4CCCOCC4)cc3)c(=O)oc2c1]

7-Isopropoxy-2-oxo-2H-Chromene-3-carboxylic acid (48.9 mg, 0.197 mmol, 1 Equiv.) in 20 mL of anhydrous dichloromethane under nitrogen was added into a 100 mL round bottom flask. (4-aminophenyl) (1,4-oxazepan-4-yl) methanone (43.39 mg, 0.197 mmol, 1 equiv.) was added into the tube followed by the addition of EDAC (75.52 mg, 0.197. mmol, 2 equiv.), Hydroxybenzotriazole (29.36 mg, 0.217 mmol, 1.1 equiv.), and N—N, Diisopropylethylamine (102.9 μL, 0.591 mmol, 3 equiv.), and the resulting reaction mixture were stirred at room temperature overnight. After stirring overnight, the solution changed colors to brownish. LC-MS and TLC (Rf: 0.31; 95% DCM: 5% Methanol) were performed to check the reaction. The reaction mixture was transferred to a separatory funnel and extracted with 10% HCL solution with water (3×50 mL). The water layer was extracted and combined organic layers were collected. The organic phase was washed with sodium bicarbonate (3×50 mL), dried with anhydrous Na₂SO₄, filtered, concentrated, and evaporated. The resultant crude compound was purified by using a 12 g silica flash column, eluted with hexanes: ethyl acetate (time/% ethyl acetate: 0/0, 2/0, 20/50, 40/100) then DCM: MeOH (time/% MeOH: 0/0, 2/0, 20/10, 30/50, 40/100). Purification by flash column chromatography afforded DDL 481. The fractions that were corresponding to the interest peaks were dried in a speed vacuum to yield (40.5 mg, Brown powder, 45.6%). ¹H NMR (400 MHz, CDCl₃) δ 10.93 (s, 1H), 8.92 (d, 1H), 7.78 (d, J=8 Hz, 2H), 7.61 (d, J=8 Hz, 1H), 7.43 (d, J=8 Hz, 2H), 6.92 (dd, J=8.7, 2.4 Hz, 1H), 6.87 (d, J=2 Hz, 1H), 4.67 (hept, J=12 Hz, 1H), 3.97-2.05 (m, 10H), 1.41 (d, J=8 Hz, 6H). LC-MS m/z [M+H]⁺ 451.25.

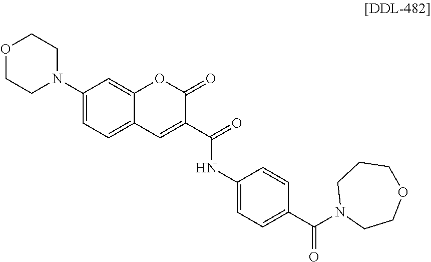 [SMILES: O=C(Nc1ccc(C(=O)N2CCCOCC2)cc1)c1cc2ccc(N3CCOCC3)cc2oc1=O]

The 2-hydroxy-4-morpholino benzaldehyde (100 mg, 0.483 mmol, 1 equiv.) and Meldrum's acid (69.6 mg), and choline chloride/urea (5 Equiv.) were taken in a pressure tube and heated at 100° C. for 3 hrs. After the completion of the reaction as indicated by TLC. The reaction mixture was poured into ice water and stirred well and the solid product obtained was filtered. The resultant crude compound was recrystallized by ethanol to afford 7-morpholine-2-oxo-2H-chromene-3-carboxylic acid (50 mg, 60.23%). ¹H NMR (400 MHz, DMSO) δ 12.65 (s, 1H), 8.59 (s, 1H), 7.67-6.81 (m, 4H), 3.70-3.67 (m, 4H), 3.40-3.38 (m, 4H) LC-MS m/z [M+H]⁺ 276.00. 7-morpholine-2-oxo-2H-chromene-3-carboxylic acid (35.74 mg, 0.13 mmol, 1 equiv.) in 15 mL of anhydrous dichloromethane was added into a 250 mL round bottom flask. (4-aminophenyl) (1,4-oxazepan-4-yl) methanone (28.63 mg, 0.13 mmol, 1 equiv.) was added into the tube followed by the addition of EDC (1-Ethyl-3-(3-dimethyl (aminopropyl)carbodiimide) (29.9 mg, 0.156 mmol, 1.2 equiv.), Hydroxybenzotriazole (19.32 mg, 0.143 mmol, 1.1 equiv.), and N—N, Diisopropylethylamine (56 μL, 0.325 mmol, 2.5 equiv.), and the resulting reaction mixture were stirred at room temperature overnight. After stirring overnight, the solution changed colors to yellowish. LC-MS and TLC (Rf 0.286; 95% methylene chloride: 5% methanol) were performed to check the reaction. The reaction mixture was transferred to a separatory funnel and extracted with 10% HCL solution with water (2×15 mL). The water layer was extracted and combined organic layers were collected. The organic phase was washed with sodium bicarbonate (2×15 mL), dried with anhydrous Na₂SO₄, filtered, concentrated, and evaporated. The resultant crude compound was purified by using a 4 g silica flash column, eluted with hexanes: ethyl acetate (time/% ethyl acetate: 0/0, 2/0, 20/95, 25/100) then DCM: MeOH (time/% MeOH: 0/0, 5/0, 20/10, 25/15, 30/100). Purification by flash column chromatography afforded DDL 482. The fractions that were corresponding to the interest peaks were dried in a speed vacuum to yield (21.02 mg, Dark yellow powder, 33.9%). ¹H NMR (400 MHz, d6-DMSO) δ 10.78 (s, 1H), 8.79 (s, 1H), 7.79-6.93 (m, 7H), 3.70-3.69 (m, 4H), 3.69-3.57 (m, 6H) 3.44-3.43 (m, 4H), 3.41-3.35 (m, 2H) 1.83-1.70 (m, 2H). LC-MS m/z [M+H]⁺ 478.58.

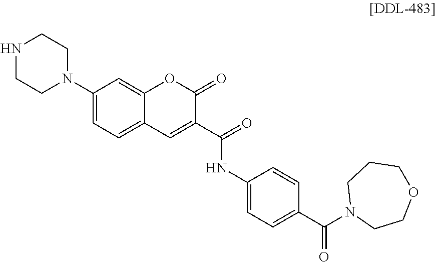 [SMILES: O=C(Nc1ccc(C(=O)N2CCCOCC2)cc1)c1cc2ccc(N3CCNCC3)cc2oc1=O]

Tert-butyl 4-(4-formyl-3-hydroxyphenyl) piperazine-1-carboxylate (100 mg, 0.326 mmol, 1 Equiv.), 2,2-dimethyl-1,3-dioxane-4,6-dione (70.48 mg, 0.489 mmol, 1.5 Equiv.) and triethylamine (14.9 μL, 0.107 mmol, 0.33 Equiv.) were combined in EtOH (1 mL). The mixture was heated at 60° C. for 4 h. The mixture was cooled to room temperature and filtered. The collected material was washed with EtOH and dried under vacuum to afford 7-(4-(tert butoxy carbonyl) piperazine-1-yl)-2-oxo-2H-chromene-3-carboxylic acid as a yellow powder (90.8 mg, Dark yellow powder, 74.4%). LC-MS m/z [M+H]⁺ 375.42. 7-(4-(tert-butoxy carbonyl) piperazine-1-yl)-2-oxo-2H-chromene-3-carboxylic acid (60.00 mg, 0.16 mmol, 1 equiv.) in 15 mL of anhydrous dichloromethane was added into a 250 mL round bottom flask. (4-aminophenyl) (1,4-oxazepan-4-yl) methanone (35.24 mg, 0.16 mmol, 1 equiv.) was added into the tube followed by the addition of EDC (1-Ethyl-3-(3-dimethyl (aminopropyl)carbodiimide) (36.8 mg, 0.192 mmol, 1.2 equiv.), Hydroxybenzotriazole (23.78 mg, 0.176 mmol, 1.1 Equiv.), and N—N, Diisopropylethylamine (69.7 μL, 0.4 mmol, 2.5 equiv.), and the resulting reaction mixture were stirred at room temperature overnight. After stirring overnight, the solution changed colors to yellowish. LC-MS and TLC (Rf: 0.58; 85% methylene chloride: 15% methanol) were performed to check the reaction. The reaction mixture was transferred to a separatory funnel and extracted with sodium bicarbonate (3×20 mL), brine wash (3×20 mL), dried with anhydrous Na₂SO₄, filtered, concentrated, and evaporated. The resultant crude compound was purified by using a 4 g silica flash column, eluted with hexanes: ethyl acetate (time/% ethyl acetate: 10/0, 30/100) then DCM: MeOH (time/% MeOH: 7/0, 35/15, 40/100). The resultant crude compound was re-purified by using a 4 g silica flash column, eluted with DCM: MeOH (time/% MeOH: 10/0, 30/15, 35/100). Re-purification by flash column chromatography afforded DDL 483. The fractions that corresponded to the interest peaks were dried in a speed vacuum to yield (25.5 mg, Yellow powder, 27.6%). LC-MS m/z [M+H]⁺ 577.50. (25.5 mg, 0.044 mmol, 1 Equiv.) was dissolved in CH₂Cl₂ (800 uL), and Trifluoroacetic acid (600 uL) was added. The reaction mixture was stirred in an ice bath for 2 hours. TLC was performed to check the reaction. CH₂Cl₂ was evaporated and the reaction mixture was dried in a rotary vacuum to yield DDL 483 (19.56 mg, 0.041 mmol, 93.2%, viscous yellow oil). ¹H NMR (400 MHz, DMSO) δ 10.76 (s, 1H), 8.81 (s, 1H), 7.84-7.03 (m, 7H), 3.69-3.64 (m, 8H), 3.57-3.43 (m, 4H), 1.83-1.70 (m, 2H), 1.49-1.19 (m, 4H).

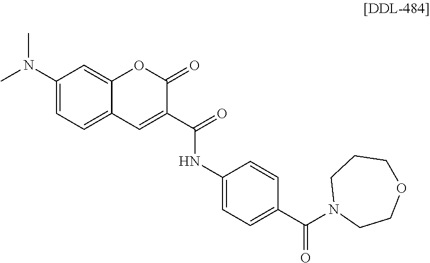 [SMILES: CN(C)c1ccc2cc(C(=O)Nc3ccc(C(=O)N4CCCOCC4)cc3)c(=O)oc2c1]

2-Hydroxy-4-dimethylamino benzaldehyde (100 mg, 0.61 mmol, 1 Equiv.), 2,2-dimethyl-1,3-dioxane-4,6-dione (133.88 mg, 0.915 mmol, 1.5 Equiv.) and triethylamine (28.29 μL, 0.203 mmol, 0.33 Equiv.) were combined in EtOH (1 mL). The mixture was heated at 60° C. for 5 h. The mixture was cooled to room temperature and filtered. The collected material was washed with EtOH and dried under vacuum to afford 7-dimethylamino-2-oxo-2H-chromene-3-carboxylic acid as a yellow powder (125.2 mg, 87.96%). LC-MS m/z [M+H]⁺ 234.25. 7-dimethylamino-2-oxo-2H-chromene-3-carboxylic acid (60.00 mg, 0.257 mmol, 1 equiv.) in 20 mL of anhydrous dichloromethane was added into a 250 mL round bottom flask. (4-aminophenyl) (1,4-oxazepan-4-yl) methanone (56.6 mg, 0.257 mmol, 1 equiv.) was added into the tube followed by the addition of EDC (1-Ethyl-3-(3-dimethyl (aminopropyl)carbodiimide) (59.04 mg, 0.308 mmol, 1.2 equiv.), Hydroxybenzotriazole (38.24 mg, 0.283 mmol, 1.1 equiv.), and N—N, Diisopropylethylamine (112 μL, 0.643 mmol, 2.5 equiv.), and the resulting reaction mixture were stirred at room temperature overnight. After stirring overnight, the solution changed colors to yellowish. LC-MS and TLC were performed to check the reaction. The reaction mixture was transferred to a separatory funnel and extracted with 10% HCL solution with water (2×20 mL). The water layer was extracted and combined organic layers were collected. The organic phase was washed with sodium bicarbonate (2×20 mL), dried with anhydrous Na₂SO₄, filtered, concentrated, and evaporated. The resultant crude compound was purified by using a 4 g silica flash column, eluted with hexanes: ethyl acetate (time/% ethyl acetate: 0/0, 5/0, 25/100) then DCM: MeOH (time/% MeOH: 0/0, 10/0, 23/10, 32/20, 40/100) gave desired product along with 7-dimethylamino-2-oxo-2H-chromene-3-carboxylic acid. Finally purified using a flash column using DCM: MeOH (time/% MeOH: 0/0, 15/0, 30/10, 35/20, 40/100) afforded DDL 484. The fractions that corresponded to the interest peaks were dried in a speed vacuum to yield (39.05 mg, Yellow powder, 34.9%). ¹H NMR (400 MHz, CDCl₃) δ 11.00 (s, 1H), 8.80 (s, 1H), 7.79-6.53 (m, 7H), 3.83-3.57 (m, 8H) 3.14 (s, 6H), 2.05-1.85 (m, 2H). LC-MS m/z [M+H]⁺ 436.75.

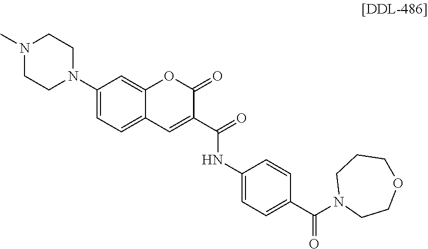 [SMILES: CN1CCN(c2ccc3cc(C(=O)Nc4ccc(C(=O)N5CCCOCC5)cc4)c(=O)oc3c2)CC1]

A solution of 4-fluoro-2-hydroxybenzaldehyde (700.55 mg, 1 Equiv.) and 1-methyl piperazine (556.47 ul, 1 Equiv.) in DMF (10 mL) was stirred at 100 degrees for 24 h. The reaction mixture was added to an aqueous potassium carbonate solution and extracted with DCM. The combined organic phases were washed with water and brine and dried over sodium sulfate. The resultant crude compound was purified by using a 4 g silica flash column, eluted with DCM: MeOH (time/% MeOH: 7/0, 20/5, 38/15). The fractions that were corresponding to the interest peaks were dried in a speed vacuum to yield 2-Hydroxy-4-(4-methyl piperazine-1-carbonyl) benzaldehyde (339.43 mg of colorless powder, 30.78%). LC-MS m/z [M+H]⁺ 221.50. 2-Hydroxy-4-(4-methyl piperazine-1-carbonyl) benzaldehyde (197.77 mg, 0.898 mmol, 1 Equiv.), 2,2-dimethyl-1,3-dioxane-4,6-dione (194.14 mg, 1.347 mmol, 1.5 Equiv.) and triethylamine (41.3 μL, 0.296 mmol, 0.33 Equiv.) were combined in EtOH (4 mL). The mixture was heated at 60° C. for 4 h. The mixture was cooled to room temperature and filtered. The collected material was washed with EtOH and dried under a vacuum to afford 7-(4-Methyl-1-piperazinyl)-2-oxo-2H-1-benzopyran-3-carboxylic acid. (284.09 mg, 0.985 mmol, 98.5%). 7-(4-Methyl-1-piperazinyl)-2-oxo-2H-1-benzopyran-3-carboxylic acid (40.00 mg, 0.139 mmol, 1 equiv.) in 15 mL of anhydrous dichloromethane, and 5 mL of DMF was added into a 250 mL round bottom flask. (4-aminophenyl) (1,4-oxazepan-4-yl) methanone (24.45 mg, 0.111 mmol, 0.8 equiv.) was added into the tube followed by the addition of EDC (1-Ethyl-3-(3-dimethyl (aminopropyl)carbodiimide) (53.48 mg, 0.278 mmol, 2 equiv.), Hydroxybenzotriazole (20.67 mg, 0.153 mmol, 1.1 equiv.), and N—N, Diisopropylethylamine (72.64 μL, 0.417 mmol, 3 equiv.), and the resulting reaction mixture were stirred for 72 h at room temperature. After stirring overnight, the solution changed colors to yellowish. FIA and TLC were performed to check the reaction. The reaction mixture was transferred to a separatory funnel and extracted with 10% HCL solution with water (2×15 mL). The water layer was extracted and combined organic layers were collected. The organic phase was washed with sodium bicarbonate (2×15 mL), dried with anhydrous Na₂SO₄, filtered, concentrated, and evaporated. The resultant crude compound was purified by using a 4 g silica flash column, eluted with DCM: MeOH (time/% MeOH: 0/0, 5/0, 15/10, 25/20, 40/100) afforded DDL 486. The fractions that were corresponding to the interest peaks were dried in a speed vacuum to yield (21.83 mg, Yellow powder, 29.86%). ¹H NMR (400 MHz, CDCl₃) δ 10.97 (s, 1H), 8.82 (s, 1H), 7.79-6.72 (m, 7H), 3.83-3.57 (m, 8H), 3.48 (t, J=4 Hz, 4H), 2.56 (t, J=4 Hz, 4H), 2.36 (s, 3H), 2.05-1.85 (m, 2H). LC-MS m/z [M+H]⁺ 491.58.

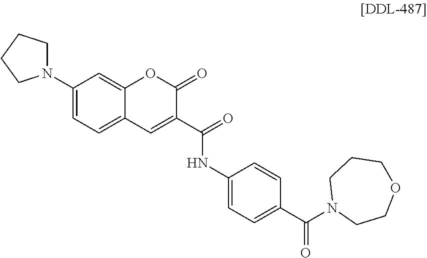 [SMILES: O=C(Nc1ccc(C(=O)N2CCCOCC2)cc1)c1cc2ccc(N3CCCC3)cc2oc1=O]

A solution of 4-fluoro-2-hydroxybenzaldehyde (1 Equiv.) and Pyrrolidine (1 Equiv.) in DMSO (5 mL) was stirred at 80 degrees for 24 h. The reaction mixture was added to an aqueous potassium carbonate solution and extracted with DCM. The combined organic phases were washed with water and brine and dried over sodium sulfate. The resultant crude compound was purified by using a 4 g silica flash column, eluted with DCM: MeOH (time/% MeOH: 5/0, 25/10, 40/20). The fractions that corresponded to the interest peaks were dried in a speed vacuum to yield 2-Hydroxy-4-(1-pyrrolidinyl) Benzyl aldehyde (425.74 mg of light pink powder, 51.53%). LC-MS m/z [M+H]⁺ 192.33. 2-Hydroxy-4-(1-pyrrolidinyl) Benzyl aldehyde (191.33 mg, 0.9 mmol, 1 Equiv.), 2,2-dimethyl-1,3-dioxane-4,6-dione (259.4 mg, 1.8 mmol, 2 Equiv.) and triethylamine (125.4 μL, 0.9 mmol, 0.33 Equiv.) were combined in EtOH (8 mL). The mixture was heated at 60° C. for 24 h. The mixture was cooled to room temperature and filtered. The collected material was washed with EtOH and dried under a vacuum to afford DDL 487 Carboxylic acid (172.01 mg, 0.663 mmol, 73.7%). DDL487 COOH (60.00 mg, 0.23 mmol, 1 Equiv.), Hydroxybenzotriazole (34.18 mg, 0.253 mmol, 1.1 Equiv.), and EDC (88.18 mg, 0.46 mmol, 2 Equiv.) were added into the round bottom flask with a stir bar. 12 mL of anhydrous DCM, 4 mL of DMF, and 3 eq. of DIPEA were added to the round bottom flask, and (4-aminophenyl)(1,4-oxazepan-4-yl)-methanone (0.8 Equiv.) was then added to the round bottom flask and stir for overnight After being stirred overnight, the solution changed colors to yellowish. FIA was performed to check the reaction. The reaction mixture was reconstituted using DCM and transferred to a separatory funnel, the water layer was extracted and combined organic layers were collected. The organic phase was washed with sodium bicarbonate (2×15 mL), and brine solution (15 mL), dried with anhydrous Na₂SO₄, filtered, concentrated, and evaporated. The resultant crude compound was purified by using a 4 g silica flash column, eluted with Hexane: Ethyl acetate (time/% Ethyl acetate: 0/0, 5/0, 25/100), and eluted with DCM: MeOH (time/% MeOH: 0/0, 5/0, 15/10, 25/20, 40/100) afforded DDL 487. The fractions that were corresponding to the interest peaks were dried in a speed vacuum to yield (55.99 mg of DDL 487, yellow powder, 52.7%). ¹H NMR (400 MHz, CDCl₃) δ 11.00 (s, 1H), 8.79 (s, 1H), 7.79-6.41 (m, 7H), 3.83-3.57 (m, 8H), 3.43 (t, J=4 Hz, 4H), 2.09 (t, J=4 Hz, 4H), 2.05-1.85 (m, 2H). LC-MS m/z [M+H]⁺ 462.83.

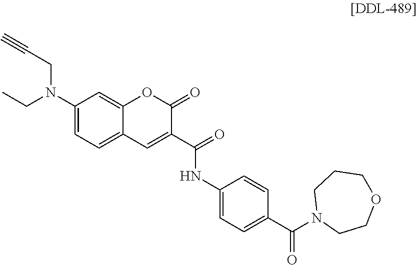 [SMILES: C#CCN(CC)c1ccc2cc(C(=O)Nc3ccc(C(=O)N4CCCOCC4)cc3)c(=O)oc2c1]

A solution of 3-(ethylamino) phenol (3.644 mmol, 1 Equiv.) and propargyl bromide (0.524 mmol, 1.2 Equiv.), and Potassium carbonate (4.3728 mmol, 1.5 Equiv.) in acetonitrile was stirred at RT for 16 hr. The reaction mixture was added to water and extracted with EtOAc. The combined organic phases were washed with water and brine and dried over sodium sulfate. The resultant crude compound was purified by using a 4 g silica flash column, eluted with Hexanes: EtOAc (time (min)/% MeOH: 2/0, 20/50, 40/100), and dried under a vacuum to afford DDL 489 Intermediate 1 (210 mg, 32.7%, brown oil). POCl₃ (0.6847 mmol, 4 Equiv.) was added dropwise to DMF on ice and the solution stirred for hr. A solution of INT 1 (0.4649 mmol, 1 Equiv.) was added and the reaction mixture was stirred at room temperature for 1 h, then heated at 75° C. for 1 h. After cooling, water was added and the mixture was stirred at room temperature, neutralized, and extracted with ethyl acetate. The organic layer was washed with brine, dried over Na₂SO₄, filtered, and the solvent evaporated. The resultant crude mixture was purified by using a 4 g silica flash column, eluted with Hexanes: EtOAc (time (min)/% EtOAc: 2/0, 20/50, 40/100) to give a DDL 489 INT2 (70.54 mg, 65.2%, light pink solid). [Intermediate 2](63.25 mg, 0.3112 mmol, 1 Equiv.), 2,2-dimethyl-1,3-dioxane-4,6-dione (67.28 mg, 0.4668 mmol, 1.5 Equiv.) and triethylamine (14.45 μL, 0.1037 mmol, 0.33 Equiv.) were combined in EtOH (3 mL). The mixture was heated at 60° C. for 24 h. The mixture was cooled to room temperature and filtered. The collected material was washed with EtOH and dried under a vacuum to afford DDL 489 carboxylic acid (57.83 mg, 68.5%, yellow solid). DDL489 COOH (55.58 mg, 0.205 mmol, 1 Equiv.) Hydroxybenzotriazole (30.47 mg, 0.2255 mmol 1.1 Equiv.), and EDC (47.13 mg, 0.2458 mmol, 1.2 Equiv.) were added into the round bottom flask with a stir bar, 10 mL of anhydrous DCM and 2.5 eq. of DIPEA were added to the round bottom flask, and (4-aminophenyl) (1,4-oxazepan-4-yl)-methanone (1 Equiv.) was then added to the round bottom flask and stir for overnight. After being stirred overnight, the solution changed colors to yellowish. Atmospheric pressure chemical ionization was performed to check the reaction. The reaction mixture was reconstituted using DCM and transferred to a separatory funnel, the water layer was extracted and combined organic layers were collected. The organic phase was washed with sodium bicarbonate (2×15 mL), and brine solution (15 mL), dried with anhydrous Na₂SO₄. filtered, concentrated, and evaporated. The resultant crude compound was purified by using a 4 g silica flash column, eluted with Hexane: Ethyl acetate (time/% Ethyl acetate: 0/0, 5/0, 25/100), and eluted with DCM: MeOH (time/% MeOH: 0/0, 5/0, 15/10, 25/20, 40/100) afforded DDL 489. The fractions that were corresponding to the interest peaks were dried in a speed vacuum to yield (50.88 mg of DDL 489, light yellow powder, 52.4%). ¹H NMR (400 MHz, CDCl₃) δ 10.99 (s, 1H), 8.84 (s, 1H), 7.80-6.70 (m, 7H), 4.14 (s, 2H), 3.84-3.66 (m, 8H), 3.6 (q, J=8 Hz, 2H), 2.30 (s, 1H), 2.05-1.85 (m, 2H), 1.31 (t, J=6 Hz, 3H), LC-MS m/z [M+H]⁺ 474.25.

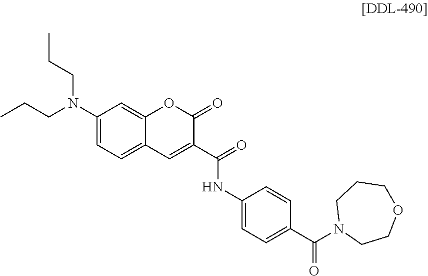 [SMILES: CCCN(CCC)c1ccc2cc(C(=O)Nc3ccc(C(=O)N4CCCOCC4)cc3)c(=O)oc2c1]

A solution of 4-fluoro-2-hydroxybenzaldehyde (1 Equiv.) Dipropyl amine (1 Equiv.) in DMSO (10 mL) was stirred at 80 degrees for 24 hr. The reaction mixture was added to an aqueous potassium carbonate solution and extracted with DCM. The combined organic phases were washed with water and brine and dried over sodium sulfate. The resultant crude compound was purified by using a 4 g silica flash column, eluted with DCM: MeOH (time/% MeOH: 7/0, 20/5, 38/15). The fractions that were corresponding to the interest peaks were dried in a speed vacuum to yield Intermediate1. (75 mg of light brown powder, 25.8%). LC-MS m/z [M+H]⁺ 222.25. Intermediate1 (75 mg, 0.3389 mmol), 2,2-dimethyl-1,3-dioxane-4,6-dione (92.8 mg, 0.644 mmol) and triethylamine (15.58 uL, 0.112 mmol) were combined in EtOH (4 mL). The mixture was heated at 60° C. for 5 h. The mixture was cooled to room temperature and filtered. The collected material was washed with EtOH and dried under a vacuum to afford Intermediate 2 (25.76 mg, 26.27%). Intermediate2 (25.5 mg, 0.0885 mmol, 1 equiv.), Hydroxybenzotriazole (13.15 mg, 0.09735 mmol, 1.1 Equiv.), and EDC (33.93 mg, 0.177 mmol, 2 Equiv.) were added to the round bottom flask with a stir bar. 5 mL of dichloromethane and 3 eq. of DIPEA were added to the round bottom flask and (4-aminophenyl) (1,4-oxazepam-4-yl)-methanone (19.49 mg, 0.0885 mmol, 1 eq.) was then added to the round bottom flask and stirred overnight. 10 ml, of 10% HCl solution was added to the separator flask, mixed thoroughly and collected organically. 10 mL of Saturated sodium bicarbonate solution was added to the separator flask, mixed thoroughly, and collected organic. Dry organic extract by adding the sodium sulfate and filter it into a beaker/dry down extract in a rotary evaporator. The resultant crude compound was purified by using a 4 g silica flash column, eluted with Hexanes: EtOAC (time/% Ethyl acetate: 5/0, 30/100), and DCM:MeOH (time/% MeOH: 7/0, 20/5, 30/15). The fractions that were corresponding to the interest peaks were dried in a speed vacuum to yield DDL490 (23.27 mg, 53.49%), ¹H NMR (400 MHz, CDCl₃) δ 11.00 (s, 1H), 8.79 (s, 1H), 7.80-6.52 (m, 7H), 3.82-3.57 (m, 8H), 3.36 (t, J=8 Hz, 4H), 1.85-1.71 (m, 2H), 1.37 (t, J=8 Hz, 4H), 0.98 (t, J=8 Hz, 6H). LC-MS m/z [M+H]⁺ 492.00.

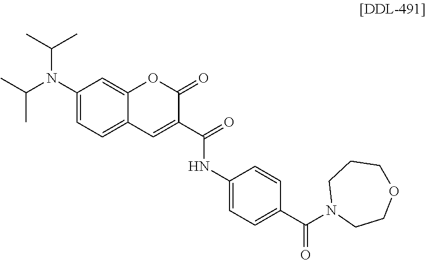 [SMILES: CC(C)N(c1ccc2cc(C(=O)Nc3ccc(C(=O)N4CCCOCC4)cc3)c(=O)oc2c1)C(C)C]

2-hydroxy-4-diisopropamino benzaldehyde: 608.79 μL (6.51 mmol) of POCl₃ were added dropwise to 5 mL of DMF cooled to 0° C. The solution was stirred 15 min at room temperature and (4.34 mmol) of N, N-Diisopropyl-3-methoxyl-aniline in 2 mL of DMF was then added. The reaction mixture was refluxed for 3 h and then quenched with water. It was basified and extracted with DCM. The combined organic layers were dried over MgSO4 and concentrated under a vacuum. The residue was purified by silica gel flash chromatography (hexane/AcOEt) to afford 2-hydroxy-4-diisopropylamine benzaldehyde (142 mg, 15%, yellow oil). ¹H NMR (400 MHz, CDCl₃) δ 11.52 (s, 1H), 9.49 (s, 1H), 7.23 (d, J=9.0 Hz, 1H), 6.42 (d, J=4 Hz, 1H), 6.42 (d, J=4 Hz, 1H) 6.22 (s, 1H), 3.99 (hept, J=6.9 Hz, 2H), 1.33 (d, J=8 Hz, 12H). LC-MS m/z [M+H]⁺ 222.17. 4-(diisopropylamine)-6-hydroxycyclohexa-1,3-diene-1-carbaldehyde (117.88 mg, 0.533 mmol, 1 Equiv.), 2,2-dimethyl-1,3-dioxane-4,6-dione (115.2 mg, 0.799 mmol, 1.5 Equiv.) and triethylamine (24.5 μL, 0.175 mmol, 0.296 Equiv.) were combined in EtOH (4 mL). The mixture was heated at 60° C. for 5 h. The mixture was cooled to room temperature and filtered. The collected material was washed with EtOH and dried under a vacuum to afford 7-(Diisopropylamino)-2-oxo-2H-1-benzopyran-3-carboxylic acid (70 mg, 49.35%, yellow powder). ¹H NMR (400 MHz, CDCl₃) δ 12.34 (s, 1H), 8.67 (s, 1H), 7.445 (d, J=6.0 Hz, 1H), 6.93 (s, 1H), 6.76 (s, 1H), 4.07 (hept, J=8 Hz, 2H), 1.39 (d, J=4 Hz, 12H). 7-(diisopropylamine)-2-oxo-2H-chromene-3-carboxylic acid (50 mg, 0.173 mmol, 1 Equiv.), HOBt (25.67 mg, 0.19 mmol, 1.1 Equiv.), and EDC (39.68 mg, 0.207 mmol, 1.2 Equiv.) were added into the round bottom flask with a stir bar. 10 mL of anhydrous DCM and 2 eq. of DIPEA were added to the round bottom flask, and (4-aminophenyl) (1,4-oxazepan-4-yl)-methanone (1 Equiv.) was then added to the round bottom flask and stir for overnight. After being stirred overnight, the solution changed colors to yellowish. Atmospheric pressure chemical ionization was performed to check the reaction. The reaction mixture was reconstituted using DCM and transferred to a separatory funnel, the water layer was extracted and combined organic layers were collected. The organic phase was washed with sodium bicarbonate (3×10 mL), and brine solution (20 mL), dried with anhydrous Na₂SO₄, filtered, concentrated, and evaporated. The resultant crude compound was purified by using a 4 g silica flash column, eluted with Hexane: Ethyl acetate (time/% Ethyl acetate: 0/0, 5/0, 25/100), and eluted with DCM: MeOH (time/% MeOH: 0/0, 5/0, 15/10, 25/20, 40/100) afforded DDL 491. The fractions that corresponded to the interest peaks were dried in a speed vacuum to yield DDL491 (50 mg, 58.8%, yellow powder). ¹H NMR (400 MHz, CDCl₃) δ 10.99 (s, 1H), 8.80 (s, 1H), 7.80-6.77 (m, 7H), 4.02 (hept, J=8 Hz, 2H), 3.83-3.57 (m, 8H), 2.05-1.84 (m, 2H), 1.38 (d, J=4 Hz, 12H). LC-MS m/z [M+H]⁺ 492.17.

### Other Examples

Compounds DDL-497, DDL-498, DDL-761, DDL-763, DDL-764, and DDL-766 to DDL-769 are prepared by the schemes below:

DDL-497 is prepared as shown below in Scheme 1:

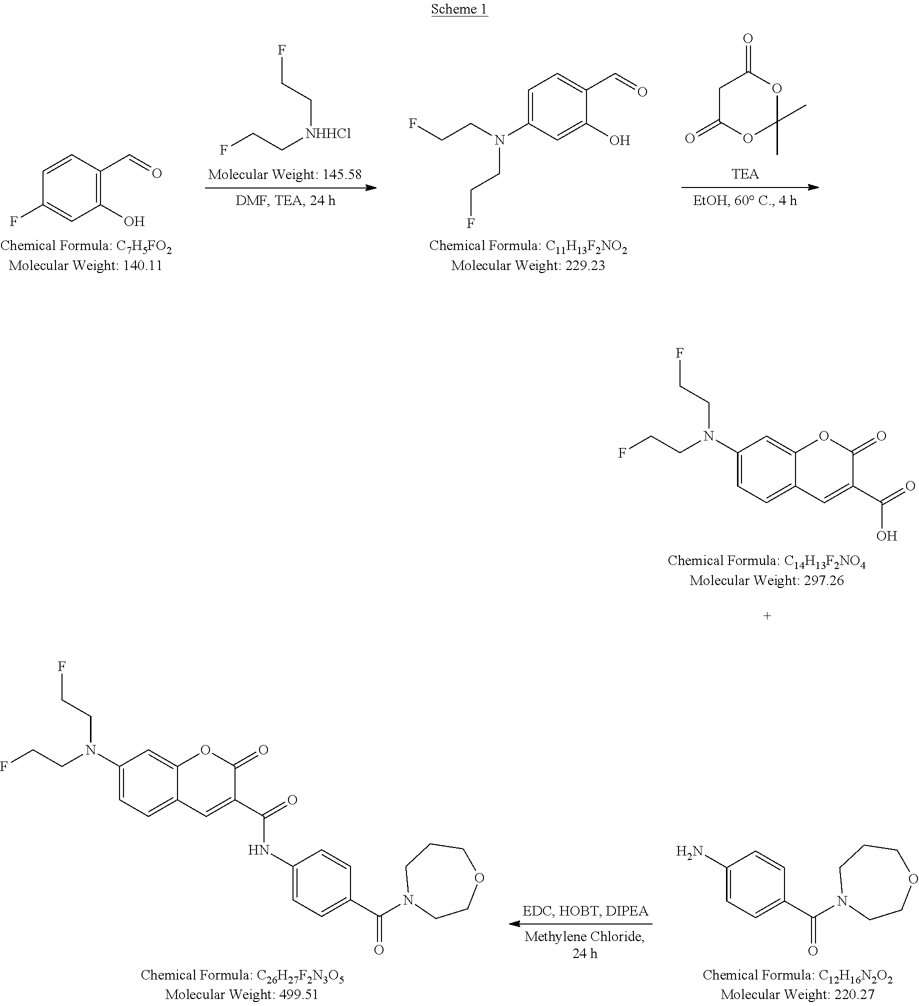

DDL-498 is prepared as shown below in Scheme 2:

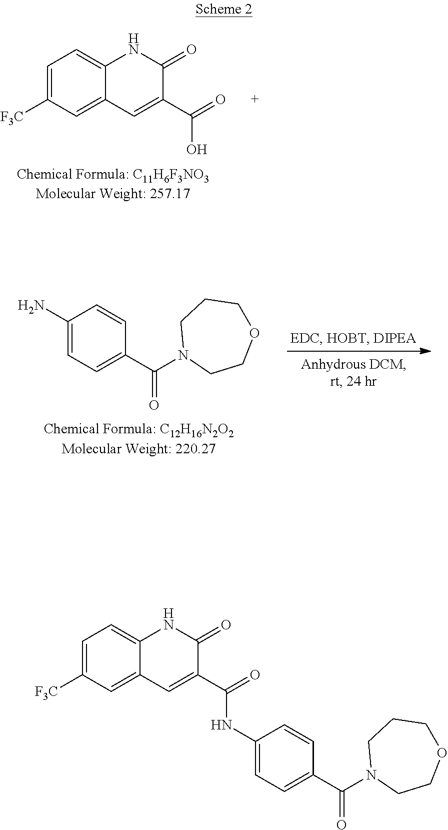

DDL-761 is prepared as shown below in Scheme 3:

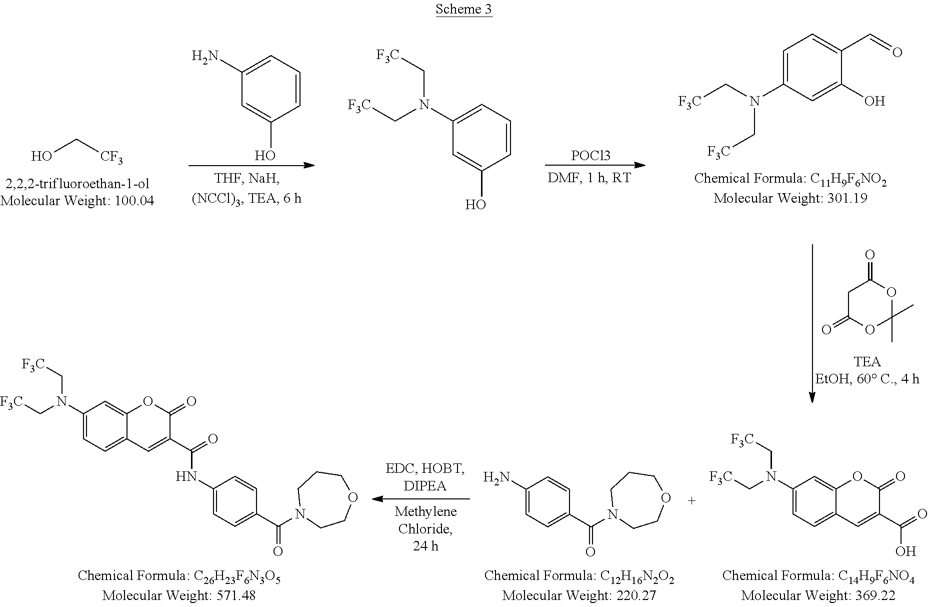

DDL-763 is prepared as shown below in Scheme 4:

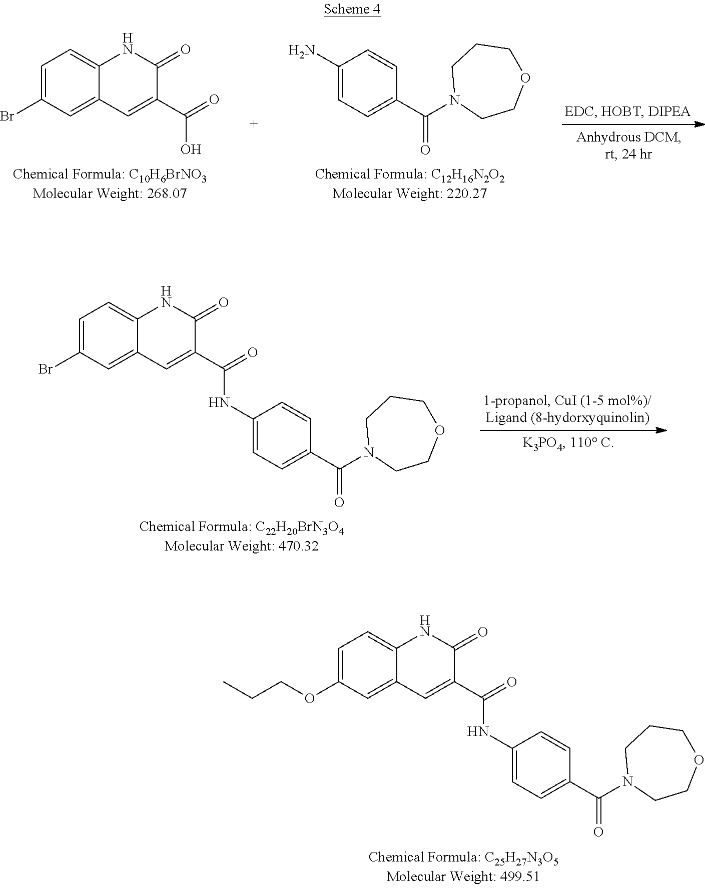 [SMILES: O=C(O)c1cc2cc(Br)ccc2[nH]c1=O ; "+" ; Nc1ccc(C(=O)N2CCCOCC2)cc1 ; O=C(Nc1ccc(C(=O)N2CCCOCC2)cc1)c1cc2cc(Br)ccc2[nH]c1=O ; CCCOc1ccc2[nH]c(=O)c(C(=O)Nc3ccc(C(=O)N4CCCOCC4)cc3)cc2c1]

DDL-764 is prepared as shown below in Scheme 5:

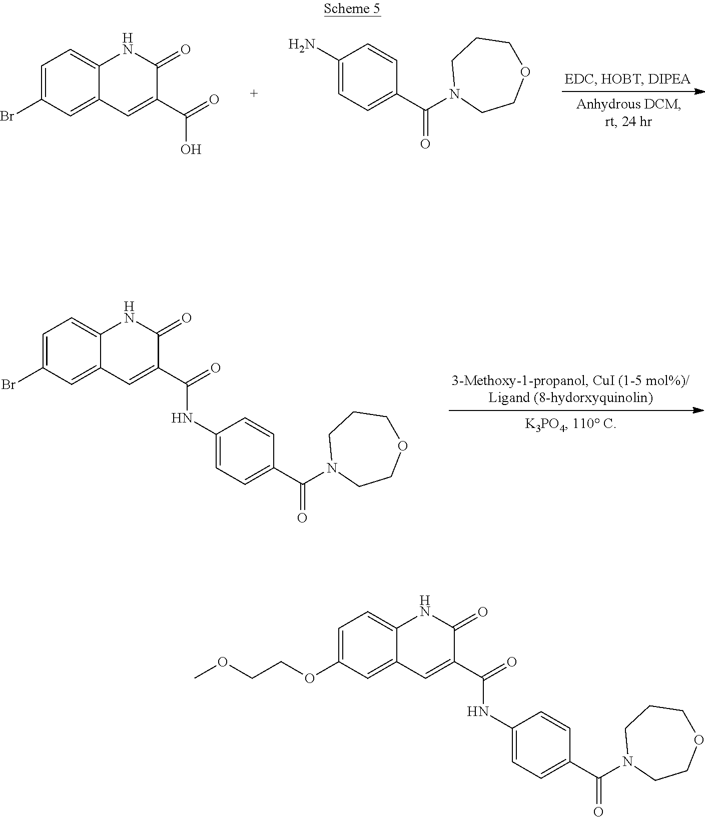

DDL-766 is prepared as shown below in Scheme 6:

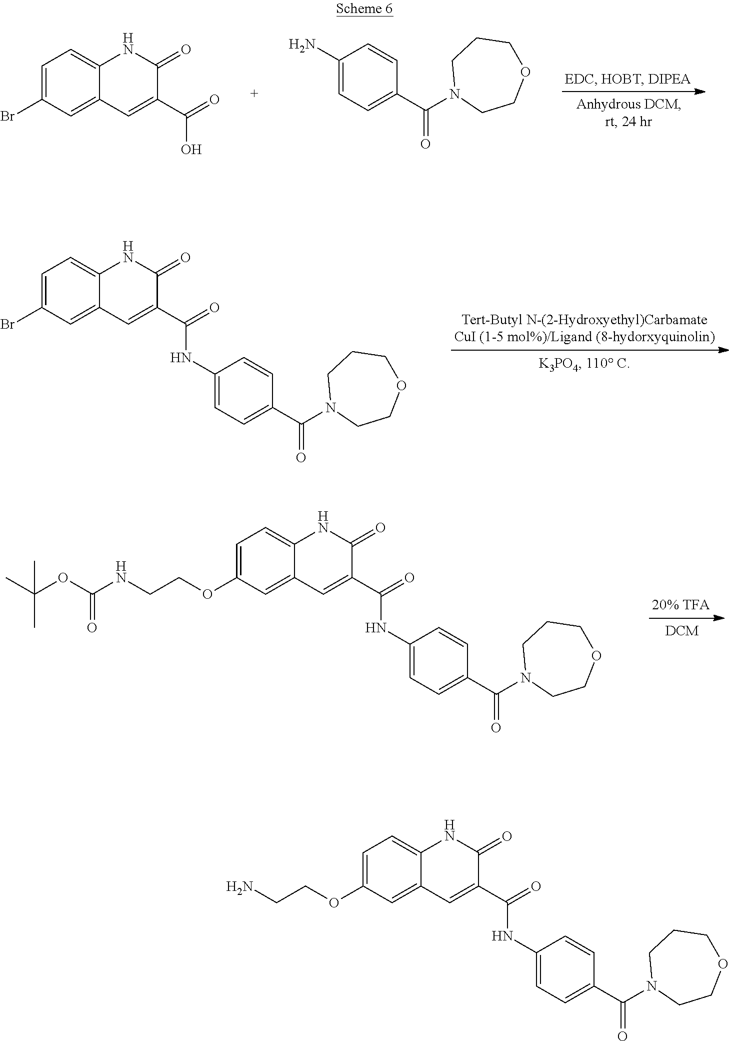

DDL-767 is prepared as shown below in Scheme 7:

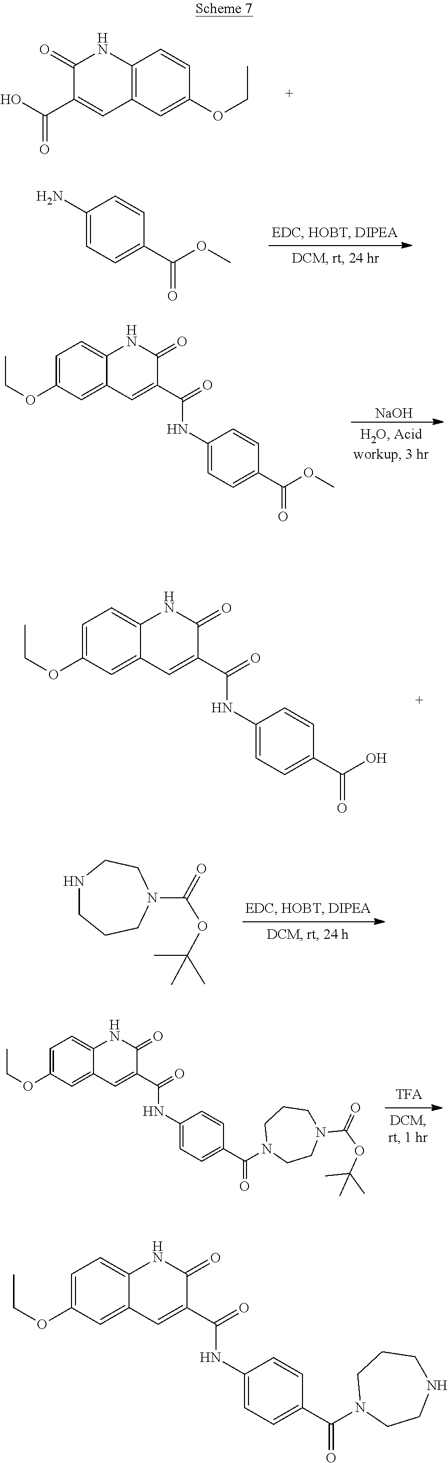

DDL-768 is prepared as shown below in Scheme 8:

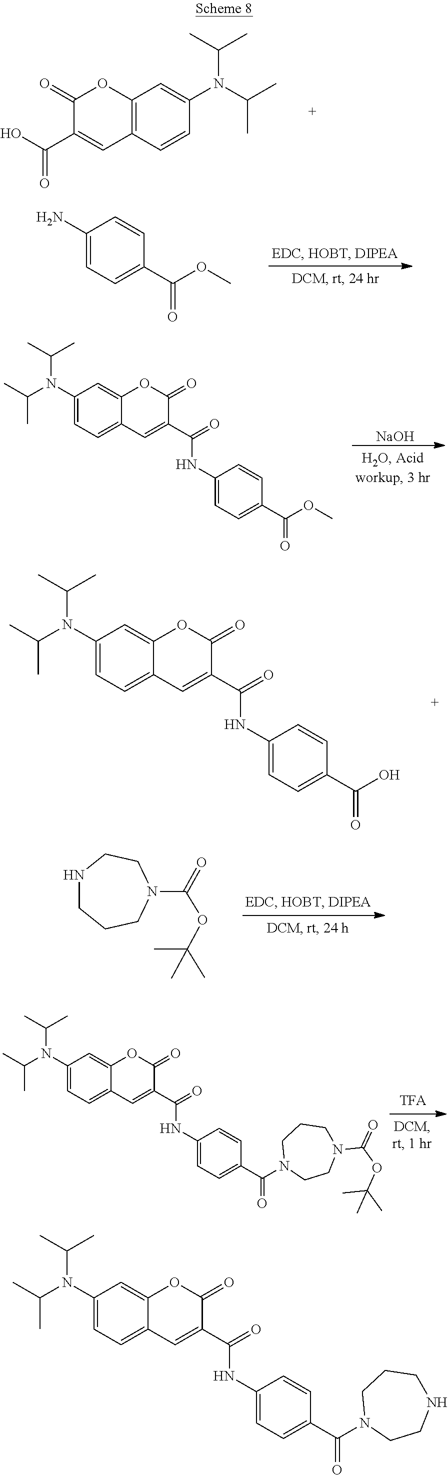

DDL-769 is prepared as shown below in Scheme 9:

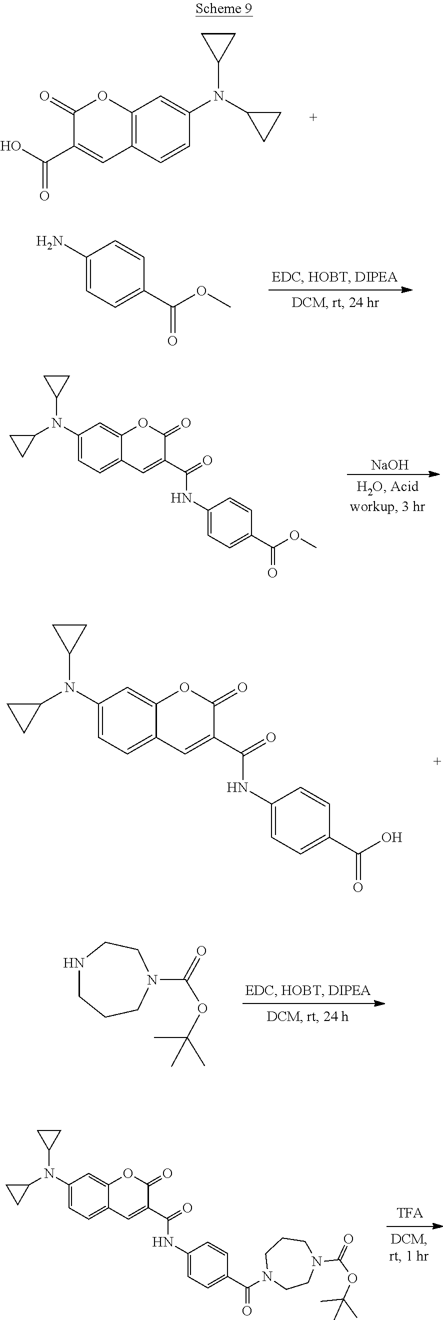

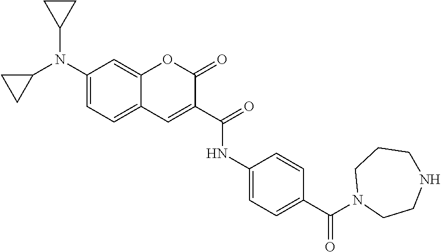 [SMILES: O=C(Nc1ccc(C(=O)N2CCCNCC2)cc1)c1cc2ccc(N(C3CC3)C3CC3)cc2oc1=O]

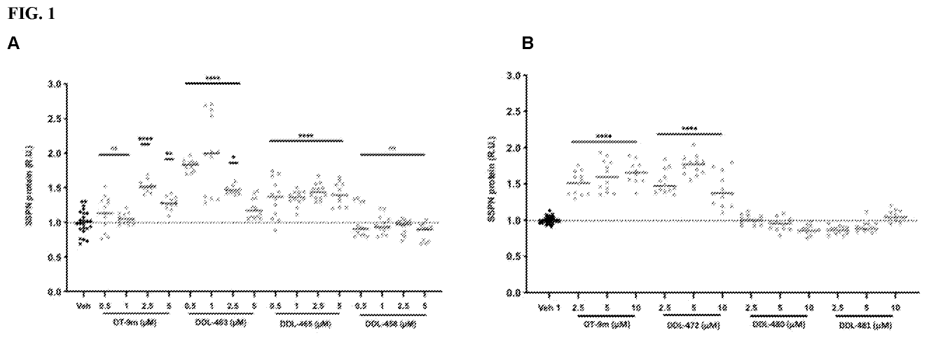

To assess activity of the new molecules, a C2C12 HiBiT assay was used as a primary screen. This assay is based on murine C2C12 cell line expressing endogenous SSPN protein with an N-term fusion protein described previously in WO 2022/055926. New analogues that exhibit activity in HiBiT assay, like DDL-463, DDL-465 and DDL-472 (FIG. 1) are subject to further evaluation in vivo for PK and PD profile.

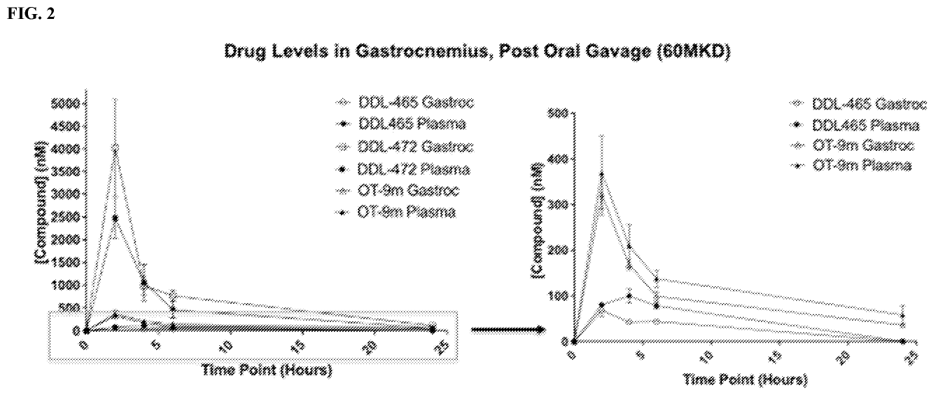

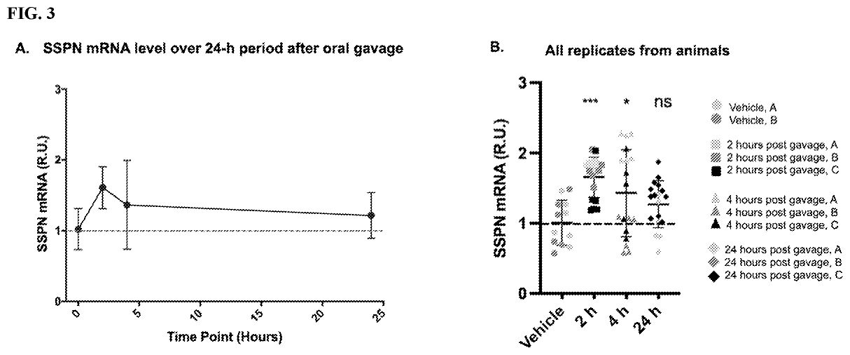

After re-screening of DDL-463, DDL-465 and DDL-472 in HiBiT assay, one quinolone series compound, DDL-465, and one coumarin series compound, DDL-472, were chosen to be tested for pharmacokinetic characteristics in C57 BL/6 mice. PK analysis of newly developed compounds revealed tenfold higher oral bioavailability of DDL-472 and threefold lower bioavailability of DDL-465 compared to the parent compound OT-9m (FIG. 2). It was found that DDL-472 increased level of SSPN transcript in C57 BL/6 mice 1.7-fold compared to vehicle-treated animals as fast as 2 hours post administration (FIG. 3).

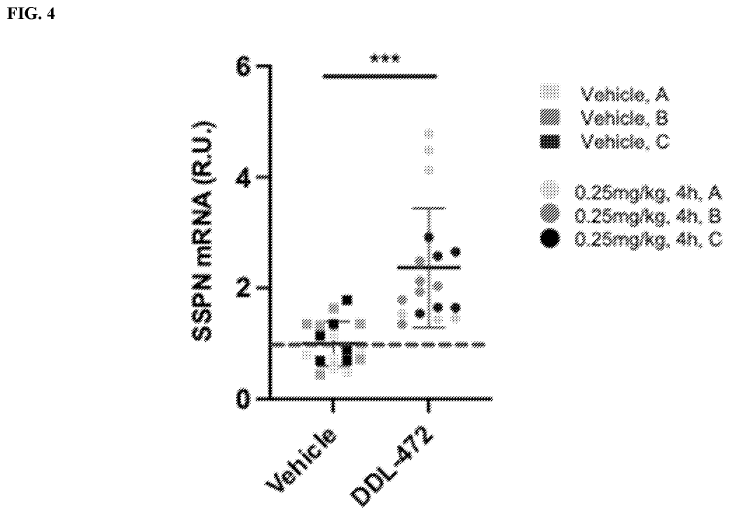

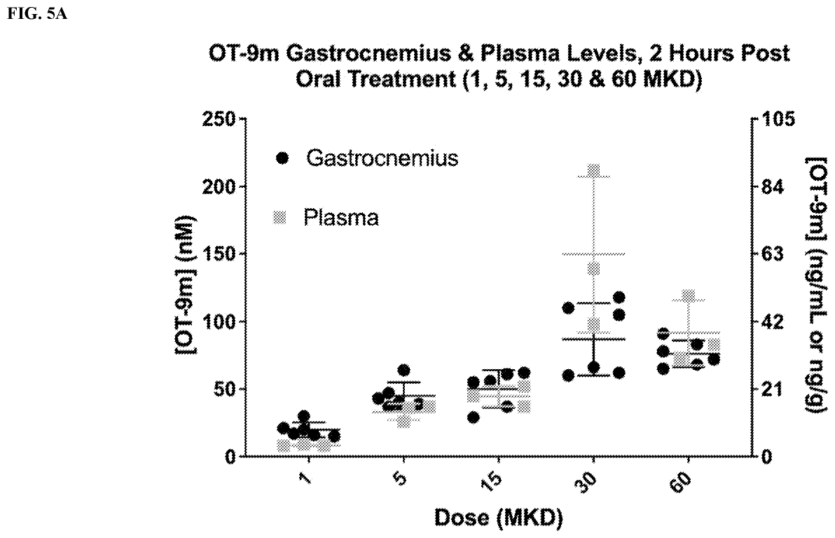

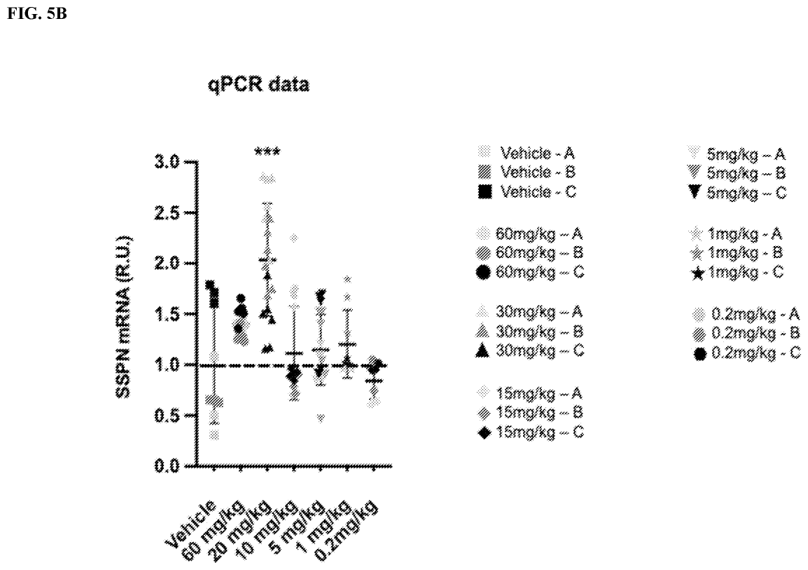

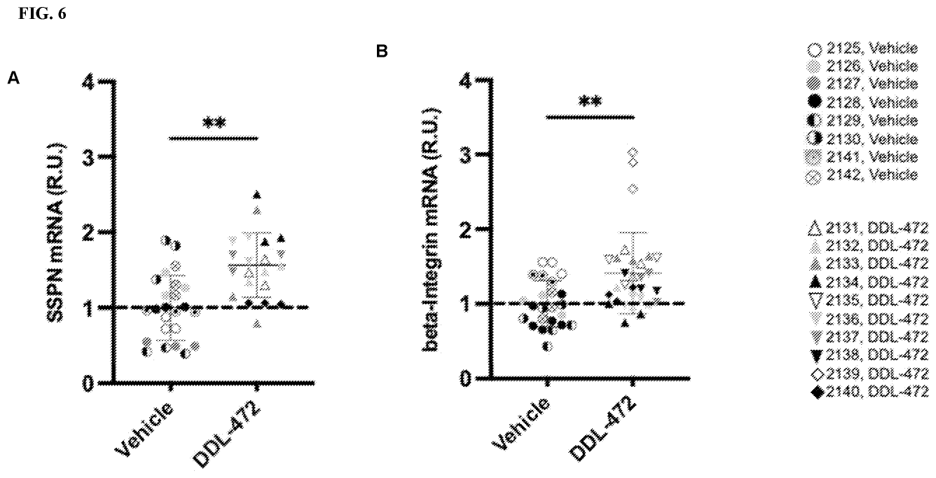

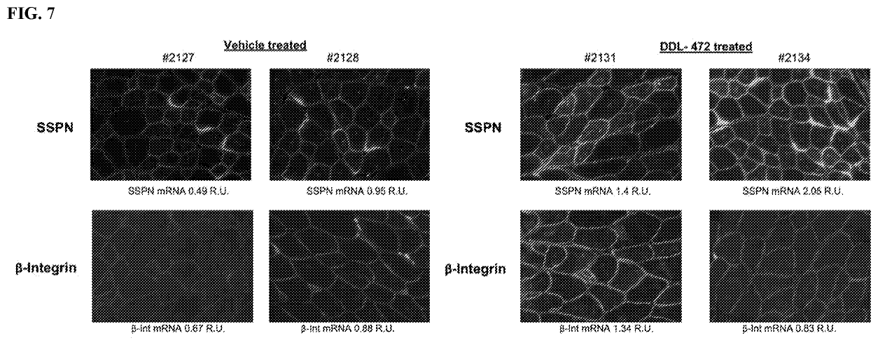

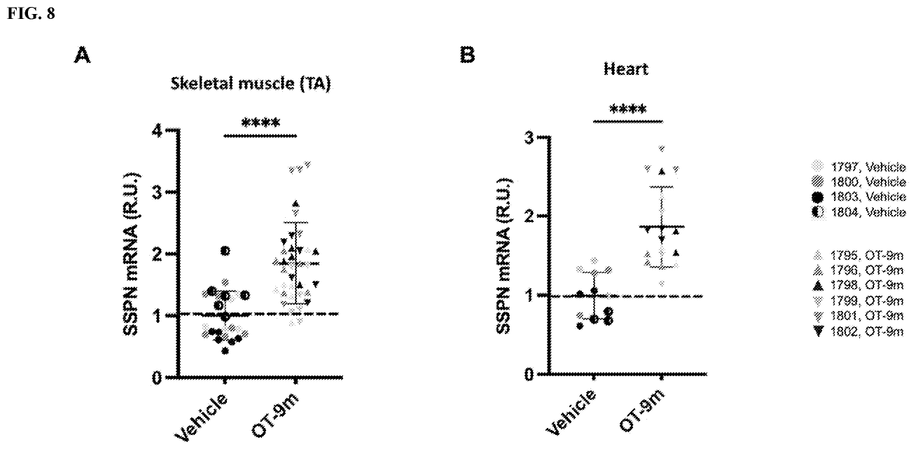

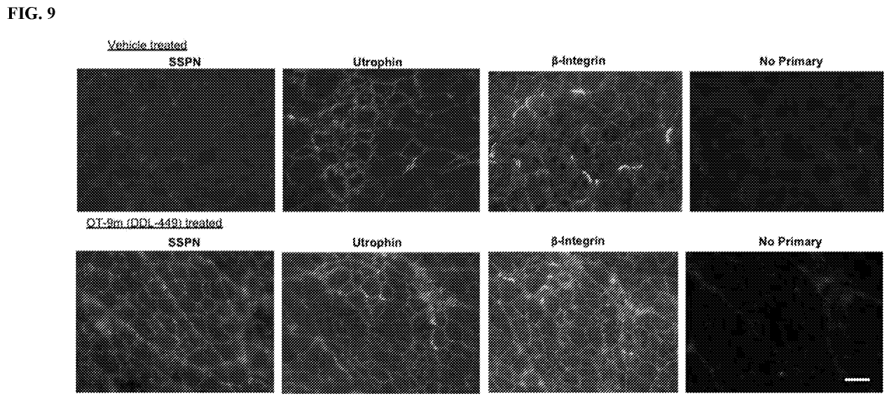

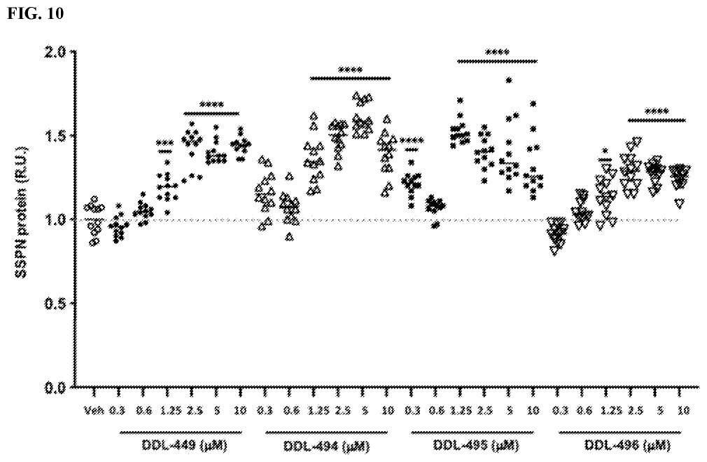

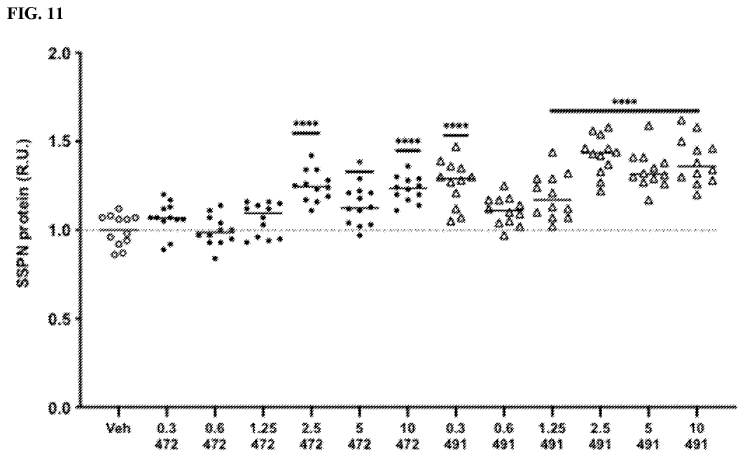

To assess ability of DDL-472 to facilitate SSPN expression in animal model of DMD, single intramuscular injection of the compound in mdx mice was performed and it was found that this compound increases the level of SSPN transcript 2-fold compared to vehicle treated mice (FIG. 4). After performing de-escalation experiment with DDL-472 in C57 BL/6 mice (FIG. 5), 30 MKD of compound for dosing of mdx mice was chosen to assess activity of this compound after systemic oral administration. It was found that oral treatment of mdx mice by DDL-472 for 3 weeks increases SSPN and β-Integrin transcript in TAs of treated mice 1.5-fold (FIG. 6). Systemic oral treatment by DDL-472 rescues sarcolemma localization of SSPN and β-Integrin in muscle cells of compound-treated mdx mice (FIG. 7). Systemic treatment with DDL-449 by oral administration of 60 MKD for 2 and 3 weeks increases SSPN transcript in skeletal muscles of treated mice up to 2.0-fold and in hearts up to 1.9-fold (FIG. 8). As in case of DDL-472, systemic oral treatment with DDL-449 rescues sarcolemma localization of SSPN, Utrophin and β-Integrin in muscle cells of compound-treated mdx mice (FIG. 9).

### In Vitro Analysis of the Compounds:

Details on development of HiBiT assay for rapid testing of new analogues in vitro (FIG. 2A, B) are provided in PCT Pub. No. WO 2022/055926.

### In Vivo Treatment of Mice:

### Oral Gavage

Systemic administration by oral gavage: 100 μl of compounds (DDL-449, DDL-465 and DDL-472) at concentrations from 0.2 to 60 mg/kg in 50% PEG-200 in water were administered directly into the stomach of C57 Bl/6 or mc mice via a technique called oral gavage. In this procedure a flexible cannula is attached to a syringe and used to deliver the compound solution into the stomach of restrained slightly sedated by isoflurane mice (FIG. 2, 3, 5).

### Pipet Feeding

Systemic compound administration by pipet feeding: 20 μl of compounds (DDL-449, DDL-472) at concentrations 20 mg/kg (DDL-472) or 60 mg/kg (DDL-449) in 50% PEG-200/15% strawberry syrup in water were administered with pipet into the mouth of mdx mice. In this procedure non-sedated mice are restrained by one hand and involuntary fed by compound using 20-200 μL pipetman (Gilson) With plastic tip. (FIG. 6-9, 16, 17).

### Intramuscular Injections (FIG. 4)

6-weeks old mdx mice were injected locally in TA muscle with 20 μl of 0.25 mg/kg of DDL-472. Muscles were harvested after 4 hours post-injections and processed for gene expression analysis. 0.25 mg/kg corresponds to 5-10 μM of local concentration of DDL-472 in TA (active concentration of DDL-472 in vitro).

### Gene Expression Analysis (FIG. 3-6, 8, 16)

Details on RNA extraction from the muscles of treated mice and gene expression analysis are provided in WO 2022/055926. Gene expression was normalized to 0-actin, mouse weight and SSPN and β-Integrin (FIG. 6, 16) transcript levels in vehicle-treated mice. Data of gene expression analysis in mice after treatment with our compounds are presented in FIG. 3-6, 8, 16.

### Pharmacokinetic Analysis (FIGS. 2 and 5A)

For FIG. 2 mice were administered DDL-472, DDL-465 and DDL-449 (OT-9m) via oral gavage at a dose of 60 mg/kg. Following compound administration, gastrocnemius tissue (left & right) and plasma were collected after euthanasia and perfusion at 2 hours. For FIG. 5A mice were administered DDL-472 via oral gavage at a dose of 0.2, 1, 5, 10, 20 & 60 mg/kg. Following compound administration, gastrocnemius tissue (left & right) and plasma were collected after euthanasia and perfusion at 2 hours.

Analysis of brain and plasma concentrations was done at the UCLA Pasarow Mass Spectrometry Lab (PMSL; Kym Faull, Ph.D., Director). Tissue samples were homogenized in a bead beater using 5 volumes of ice-cold 80% acetonitrile (1/5; mg of tissue/uL of 80% ACN). Plasma analytes were extracted using 4 volumes of ice-cold acetonitrile (1/4; uL of plasma/uL of ACN). Solutions were clarified by centrifugation (16,000×g, 5 min) and the supernatants were transferred to new tubes and lyophilized. Samples were reconstituted in 100 μL of 50/50/0.1 (Water/Acetonitrile/Formic Acid) prior to analysis via liquid chromatography-tandem mass spectrometry (LC-MS/MS).

A targeted LC-MS/MS assay was developed for each compound using the multiple reaction monitoring (MRM) acquisition method on a 6460 triple quadrupole mass spectrometer (Agilent Technologies) coupled to a 1290 Infinity HPLC system (Agilent Technologies) with a Phenomenex analytical column (Kinetex 1.7 μm C18 100 Å 100×2.1 mm). The HPLC method utilized a mixture of solvent A (99.9/1 Water/Formic Acid) and solvent B (99.9/1 Acetonitrile/Formic Acid) and a gradient was use for the elution of the compounds (min/% B: 0/20, 3/20, 19/99, 20/99, 21/20, 30/20). In this assay, detection of fragmented ions originating from each compound at specific LC retention times were utilized to ensure specificity and accurate quantification in the complex biological samples.

An internal standard (IS; reserpine) was added to every sample to account for compound loss during sample processing. Standards were made in drug naïve plasma and gastrocnemius lysates with increasing amounts of tested compound (S1, S2: 0 pmol/S3, S4: 1 pmol/S5,S6: 10 pmol/S7,S8: 100 μmol, S9,S10: 1000 μmol). The standard curve was made by plotting the amount of each compound per standard vs. the ratio of measured chromatographic peak areas corresponding to a compound (compound/IS). The trendline equation was then used to calculate the absolute concentrations of tested compounds in plasma and gastrocnemius tissue.

### Indirect Immunofluorescent Analysis (FIGS. 7 and 9)

Tissue was frozen in liquid nitrogen cooled isopentane and mounted on OCT (Tissue Tek) and stored at −80° C. To inhibit non-specific interactions, transverse cryosections (10 μm thick) were blocked with 3% BSA at room temperature, followed by incubation with avidin/biotin blocking kit (Vector Laboratories). Sections were washed with PBS and primary antibodies (anti-SSPN rabbit monoclonal antibody 10B8 (Crosbie Lab), β-Integrin antibody (EMD Millipore #MAB1900), Utrophin mouse monoclonal antibody MANCHO3 (DSHB)) were detected with species specific biotinylated antibodies (anti-mouse or anti-rabbit) for 1 hour at room temperature. Bound antibodies were visualized by incubation with Fluorescein-conjugated Avidin D (Vector Laboratories). Slides were wet mounted in Vectashield with or without DAPI (Vector Laboratories) before analysis by microscopy on Zeiss Axio Observer 7 or Axio Imager M2 (Carl Zeiss). Images were captured with Hammatsu ORCA-Flash 4.0 V3 digital complementary metal oxide semiconductor camera and either EC Plan-Neofluar 10×/0.30 Ph1 or Plan-Aprochromat 20×/0.8 M27 objectives. All measurements were performed using Image J software (NIH) or Zen (Carl Zeiss).

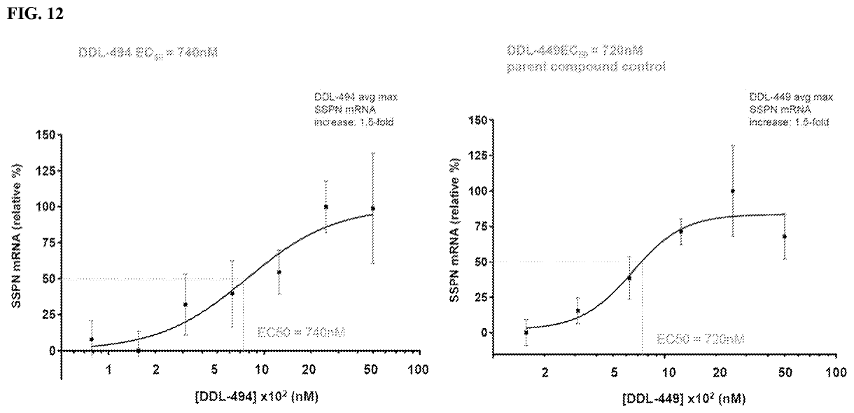

### Culturing of Mouly DMD Cells for EC₅₀ Determination of DDL-449, -494, -472, -491 (FIG. 12, 14).

For proliferation of the Mouly DMD myoblasts, cells were cultured in skeletal muscle growth medium (Skeletal Muscle Growth Medium (Promocell) and 1% penicillin-streptomycin)

at 37° C. with 5% CO₂. Upon reaching 90% confluence, the growth medium was replaced with skeletal muscle differentiation medium (Skeletal Muscle Differentiation Medium (Promocell) and 1% penicillin-streptomycin) and cultured at 37° C. with 5% CO₂. For determination of EC₅₀, cells were treated with the compounds at the concentrations 5000, 2500, 1250, 625, 312, and 156 nM or vehicle (0.5% DMSO) on day 2 of differentiation and harvested on day 4 of differentiation.

RNA isolation and purification was performed using the Direct-zol RNA Miniprep Plus RNA extraction kit (Zymo Research). RNA concentrations was determined using NanoDrop Lite (Thermo Fisher Scientific) and 750 ng of RNA was added into a 20 μl reaction of the iScript cDNA synthesis kit (Bio-Rad) with the following cycling conditions: 25° C. for 5 min, 42° C. for 30 min, 85° C. for 5 min. To quantify human sspn mRNA, TaqMan assay (assay ID Hs01025520m_1) and for β-actin (assay ID Hs01060665_g1) (Thermo Fisher Scientific) were used with the following reaction conditions: 50° C. for 2 min, 95° C. for 10 min, 40 cycles of 95° C. for 15 s and 62° C. for 1 min. qPCR data were analysed using the ddCT method. Sspn gene expression was normalized to housekeeping gene β-actin and sspn mRNA levels in vehicle-treated cells (relative expression of sspn mRNA in vehicle control=1). To generate EC₅₀ plot, sspn gene expression was represented as percentage increase normalized to the starting value.

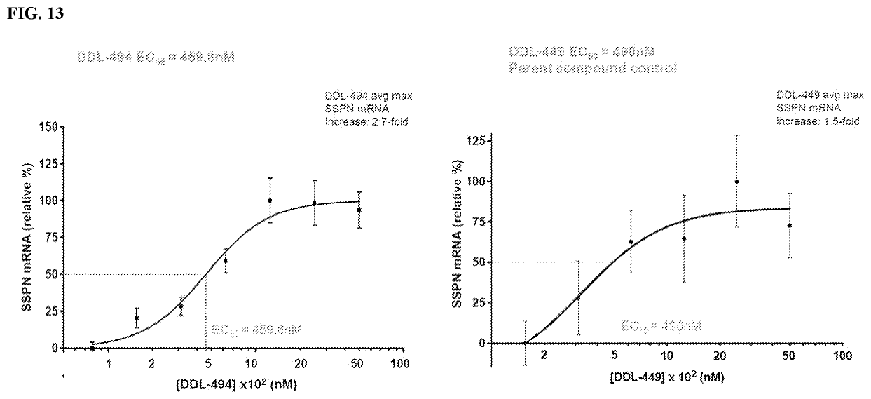

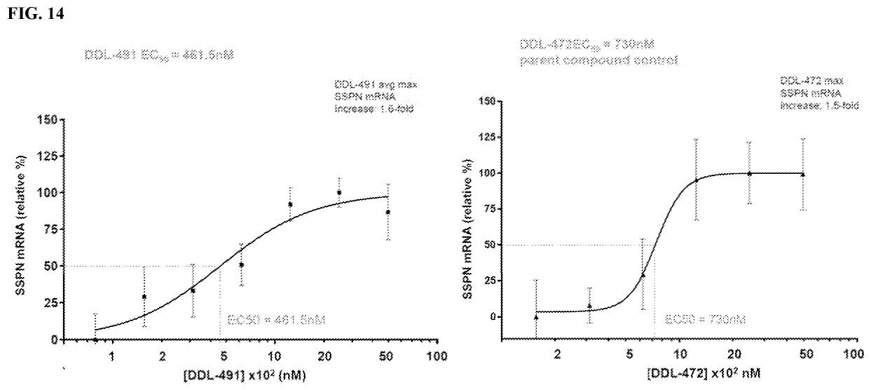

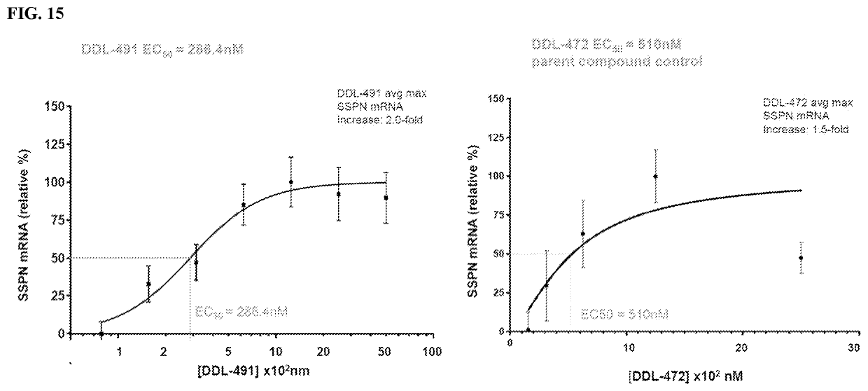

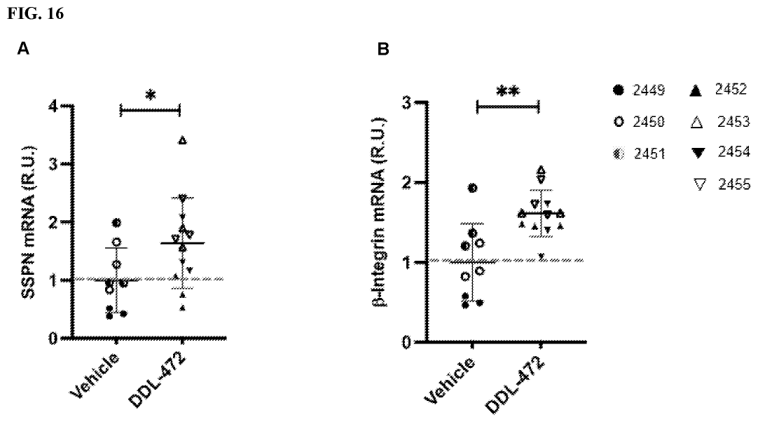

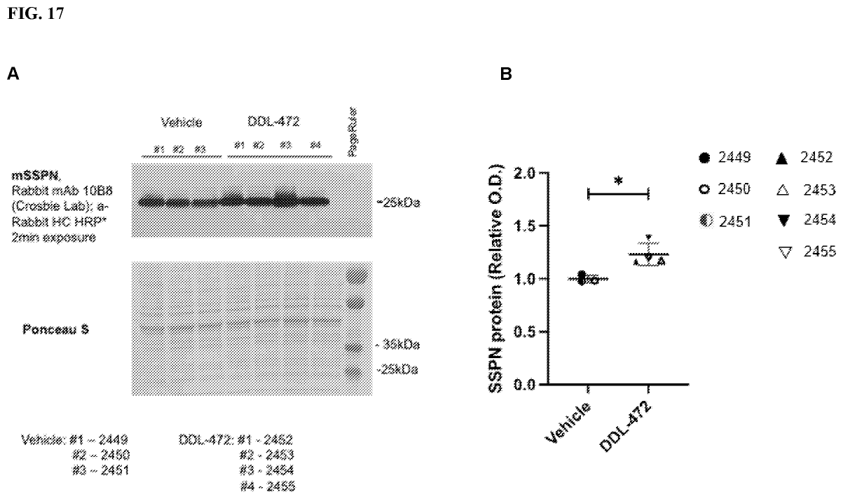

### Culturing of H2K mdx Cells for EC₅₀ Determination of DDL-449, -494, -472, -491 (FIG. 13, 15).

For proliferation of conditionally immortalized H2K mdx myoblasts, cell culture flasks were coated with 0.01% gelatin in DMEM (Gibco) for 30 min at 37° C. Gelatin was aspirated and cells added in H2K growth medium: DMEM+20% fetal bovine serum (HiClone), 2% L-glutamine, 2% chicken embryo extract, and 1% penicillin-streptomycin. Cells were cultured at 33° C. with 5% CO₂. For differentiation of H2K mdx myoblasts, cell culture plates were coated with 0.1 mg/mL of Matrigel in DMEM for 1 h at 37° C. Matrigel aspirated, cells were added in the H2K growth medium and cultured for 2-3 days at 33° C. with 5% CO₂. Upon reaching 90% confluence, medium was changed to H2K differentiation medium (DMEM+2% horse serum (Sigma), 2% L-glutamine, and 1% penicillin-streptomycin). Cells were cultured at 37° C. with 5% CO₂. For determination of EC₅₀, cells were treated with the compounds at the concentrations 5000, 2500, 1250, 625, 312, and 156 nM or vehicle (0.5% DMSO) on day 2 of differentiation and harvested on day 4 of differentiation.

RNA isolation and purification was performed using the Direct-zol RNA Miniprep Plus RNA extraction kit (Zymo Research). RNA concentrations was determined using NanoDrop Lite (Thermo Fisher Scientific) and 750 ng of RNA was added into a 20 μl reaction of the iScript cDNA synthesis kit (Bio-Rad) with the following cycling conditions: 25° C. for 5 min, 42° C. for 30 min, 85° C. for 5 min. For mouse sspn gene expression, a reaction containing SsoFast EvaGreen Supermix, 400 nM of each primer listed in Table below, and cDNA corresponding to 37.5 ng RNA was set up. qPCR analysis was performed using the QuantStudio 5 Real-Time PCR System (Applied Biosystems) using the following amplification conditions: 55° C. for 2 min, 95° C. for 2 min, 40 cycles of 95° C. for 10 s, and 62° C. for 30 s. Data were analysed using the ddCT method, sspn gene expression was normalized to housekeeping gene β-actin and sspn mRNA levels in vehicle-treated cells (relative expression of sspn mRNA in vehicle control=1). To generate EC₅₀ plot, sspn gene expression was represented as percentage increase normalized to the starting value.

### Primers Used for Gene Expression Analysis

| | | LOCA- | AMPLICON | |
|---|---|---|---|---|
| PRIMER | SEQUENCE (5′ → 3′) | TION | LENGTH | AE |
| SSPN F | TGCTAGTCAGAGATACTCCGTTC | exons | 103 bp | 94% |
| SSPN R | GTCCTCTCGTCAACTTGGTATG | 1-2 | | |
| ACTB F | TCCTGACCCTGAAGTACCCCAT | exons | 131 bp | 104% |
| ACTB R | CTCGGTGAGCAGCACAGGGT | 1-2 | | |

### Immunoblotting for Detection of SSPN in Skeletal Muscle Protein Lysates (FIG. 17)

We analyzed SSPN protein by immunoblotting of total muscle lysates from quadriceps of mdx (C57BL/10ScSn-Dmd mdx/J), treated with compound (DDL-472) or vehicle. Mice maintained following guidelines established by the Institutional Animal Care and Use Committee at the University of California, Los Angeles, and approval for the mice in this study was granted by the UCLA Institutional Animals Care and Use Committee (IACUC) (#2000-029). Quadriceps muscles were harvested after 3 weeks of treatment (day 22), snap-frozen in liquid nitrogen, pulverized under liquid nitrogen, and homogenized on ice in 30× volume of RIPA buffer containing 50 mM Tris-HCl, pH 7.4, 150 mM NaCl, 1% Triton X-100, 0.5% Na deoxycholate (DOC), 0.1% sodium dodecyl sulfate (SDS) with protease inhibitor cocktail (Thermo Fisher Scientific), and phosphatase inhibitor cocktail (Thermo Fisher Scientific). Lysates were incubated for one hour at 4° C. with rotation and clarified by centrifugation at 18,000×g for 15 mins. Protein concentrations of clarified supernatants were quantified using the DC Protein Assay (Bio-Rad). Supernatants were separated by SDS-electrophoresis (20 μg of total protein) on 12% Bolt Bis-Tris gels (Thermo Fisher Scientific) in MOPS SDS Running Buffer (Invitrogen). PageRuler Plus Prestained Protein Ladder (Thermo Fisher Scientific) was used as a molecular weight marker. Electrophoresis was performed for 2 hours at 20 mA per gel at room temperature. Transfer from the gel to the nitrocellulose membrane (Li-Cor Biosciences) was performed by wet transfer method at 4° C. for 2 hours at constant 100V. After transfer, membranes were quickly rinsed with deionized water, stained with Ponceau S for visualization of total protein loading. Ponceau S were washed off the membrane with Tris-buffer, pH 7.4 (Thermo Fisher Scientific). Non-specific binding was blocked by incubation of the membranes in blocking buffer (5% nonfat dry milk (Nestle Carnation) in Tris-buffer, pH 7.4 with 0.1% Tween-20 (TBS-T)) followed by incubation with primary antibodies (anti-SSPN rabbit monoclonal antibody 10B8 (Crosbie Lab)) overnight at 4° C. with gentle rotation. Membranes were washed three times, 5 min each in TBS-T before applying secondary antibody, horseradish peroxidase-conjugated anti-rabbit IgG (Abcam) 1:30,000 in blocking buffer. The signal was detected with SuperSignal West Pico Plus Chemiluminescence kit (Thermo Fisher Scientific) on Blue Basic Autoradiography Film (GeneMate).

Quinolone Series

| | | | SSPN -HiBiT fold change |
|---|---|---|---|
| DDL# | Structure | M.W. | over vehicle |
| DDL-458 | 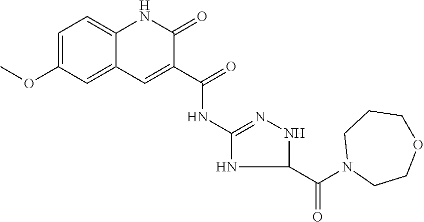 [SMILES: COc1ccc2[nH]c(=O)c(C(=O)NC3=NNC(C(=O)N4CCCOCC4)N3)cc2c1] | 414.17 | ~1 |
| DDL-459 | 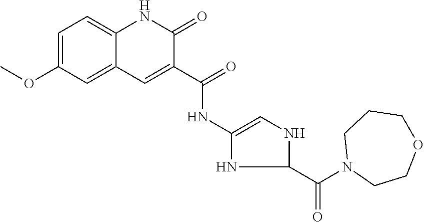 [SMILES: COc1ccc2[nH]c(=O)c(C(=O)NC3=CNC(C(=O)N4CCCOCC4)N3)cc2c1] | 413.17 | ~1 |
| DDL-460 | 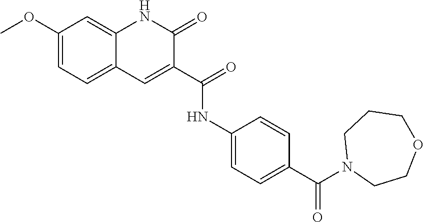 [SMILES: COc1ccc2cc(C(=O)Nc3ccc(C(=O)N4CCCOCC4)cc3)c(=O)[nH]c2c1] | 421.45 | 1.1 |
| DDL-461 | 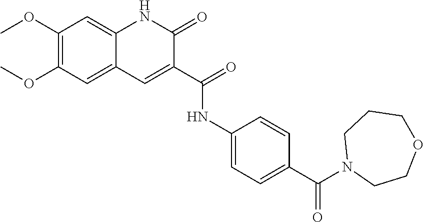 [SMILES: COc1cc2cc(C(=O)Nc3ccc(C(=O)N4CCCOCC4)cc3)c(=O)[nH]c2cc1OC] | 451.47 | 1.1 |
| DDL-462 | 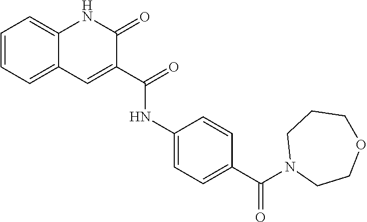 [SMILES: O=C(Nc1ccc(C(=O)N2CCCOCC2)cc1)c1cc2ccccc2[nH]c1=O] | 391.41 | ~1 |
| DDL-463 | 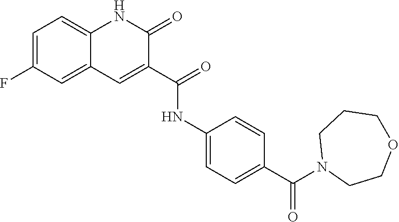 [SMILES: O=C(Nc1ccc(C(=O)N2CCCOCC2)cc1)c1cc2cc(F)ccc2[nH]c1=O] | 409.41 | 1.4 |
| DDL-464 | 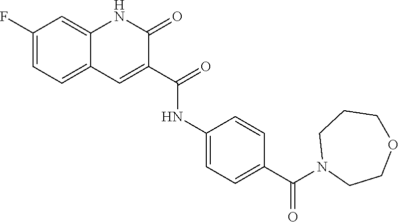 [SMILES: O=C(Nc1ccc(C(=O)N2CCCOCC2)cc1)c1cc2ccc(F)cc2[nH]c1=O] | 409.41 | ~1 |
| DDL-465 | 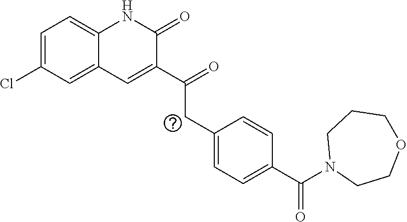 [SMILES: O=C(Cc1ccc(C(=O)N2CCCOCC2)cc1)c1cc2cc(Cl)ccc2[nH]c1=O] | 425.86 | 1.4 |
| DDL-467 | 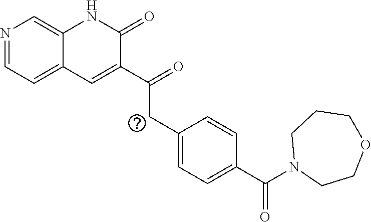 [SMILES: O=C(Cc1ccc(C(=O)N2CCCOCC2)cc1)c1cc2ccncc2[nH]c1=O] | 392.41 | 1.2 |
| DDL-469 | 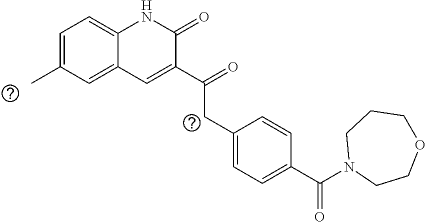 [SMILES: Cc1ccc2[nH]c(=O)c(C(=O)Cc3ccc(C(=O)N4CCCOCC4)cc3)cc2c1] | 436.42 | 1.2 |
| DDL-475 | 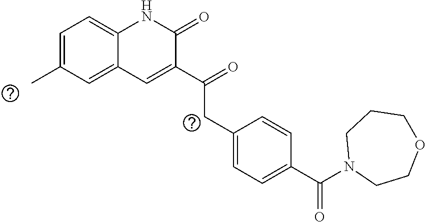 [SMILES: Cc1ccc2[nH]c(=O)c(C(=O)Cc3ccc(C(=O)N4CCCOCC4)cc3)cc2c1] | 406.44 | ~0.9 |
| DDL-492 | 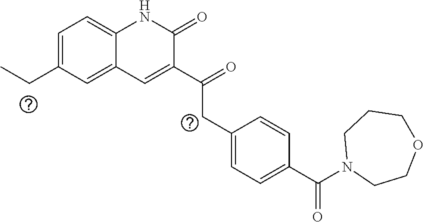 [SMILES: CCc1ccc2[nH]c(=O)c(C(=O)Cc3ccc(C(=O)N4CCCOCC4)cc3)cc2c1] | 420.47 | ~0.9 |
| DDL-493 | 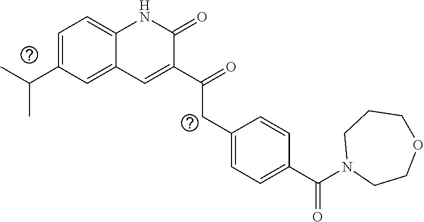 [SMILES: CC(C)c1ccc2[nH]c(=O)c(C(=O)Cc3ccc(C(=O)N4CCCOCC4)cc3)cc2c1] | 434.20 | N/A |
| DDL-494 | 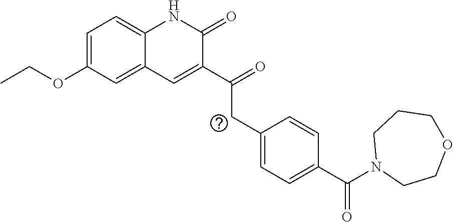 [SMILES: CCOc1ccc2[nH]c(=O)c(C(=O)Cc3ccc(C(=O)N4CCCOCC4)cc3)cc2c1] | 421.6 | 1.5 |
| DDL-495 | 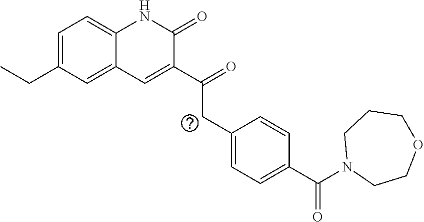 [SMILES: CCc1ccc2[nH]c(=O)c(C(=O)Cc3ccc(C(=O)N4CCCOCC4)cc3)cc2c1] | 419.18 | 1.3 |
| DDL-496 | 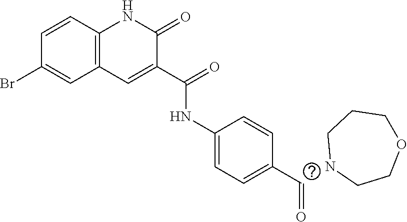 | 470.32 | 1.3 |
| DDL-498 | 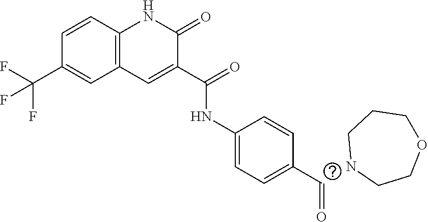 | 459.43 | N/A |
| DDL-762 | 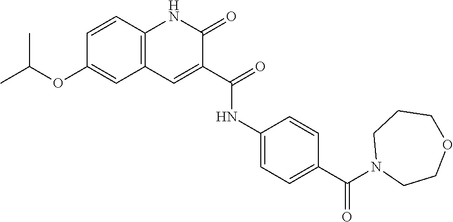 [SMILES: CC(C)Oc1ccc2[nH]c(=O)c(C(=O)Nc3ccc(C(=O)N4CCCOCC4)cc3)cc2c1] | 449.51 | 1.4 |
| DDL-763 | 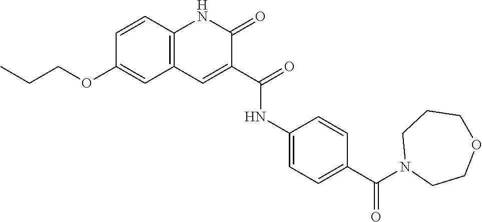 [SMILES: CCCOc1ccc2[nH]c(=O)c(C(=O)Nc3ccc(C(=O)N4CCCOCC4)cc3)cc2c1] | 449.51 | N/A |
| DDL-764 | 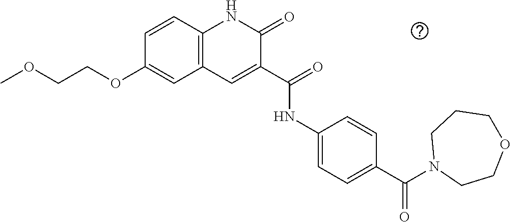 [SMILES: COCCOc1ccc2[nH]c(=O)c(C(=O)Nc3ccc(C(=O)N4CCCOCC4)cc3)cc2c1] | 465.51 | N/A |
| DDL-766 | 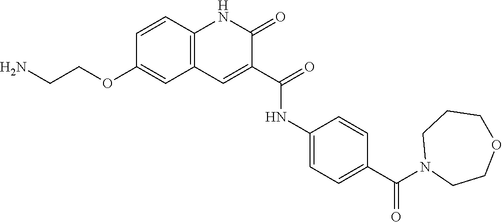 [SMILES: NCCOc1ccc2[nH]c(=O)c(C(=O)Nc3ccc(C(=O)N4CCCOCC4)cc3)cc2c1] | 450.50 | N/A |
| DDL-767 | 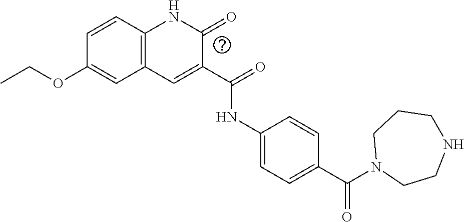 [SMILES: CCOc1ccc2[nH]c(=O)c(C(=O)Nc3ccc(C(=O)N4CCCNCC4)cc3)cc2c1] | 434.50 | N/A |
| Coumarin Series | | | |
| DDL-470 | 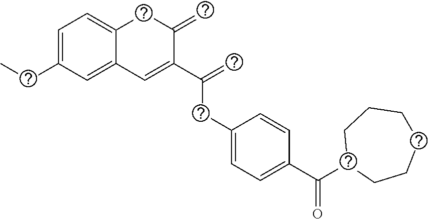 [SMILES: COc1ccc2c(c1)C=C(C(=O)Cc1ccc(C(=O)N3CCCOCC3)cc1)C(=O)C2] | 422.44 | ~1 |
| DDL-471 | 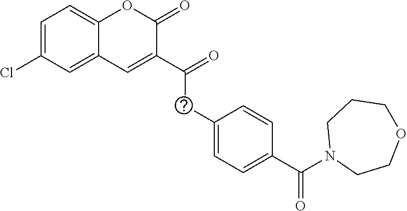 [SMILES: O=C(Cc1ccc(C(=O)N2CCCOCC2)cc1)c1cc2cc(Cl)ccc2oc1=O] | 426.85 | 1.1 |
| DDL-472 | 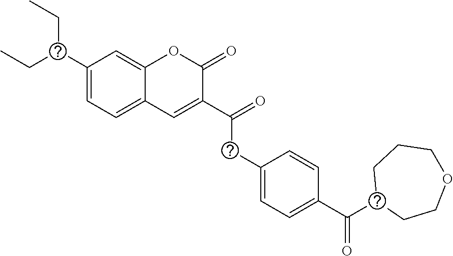 [SMILES: CCC(CC)c1ccc2cc(C(=O)Cc3ccc(C(=O)N4CCCOCC4)cc3)c(=O)oc2c1] | 463.53 | 1.4 |
| DDL-473 | 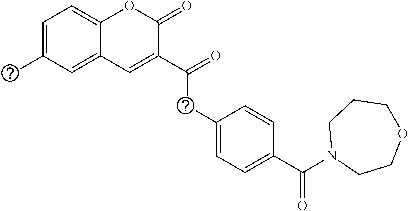 [SMILES: Cc1ccc2oc(=O)c(C(=O)Cc3ccc(C(=O)N4CCCOCC4)cc3)cc2c1] | 471.31 | 1.1 |
| DDL-474 | 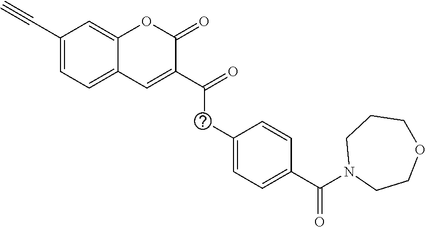 [SMILES: C#Cc1ccc2cc(C(=O)Cc3ccc(C(=O)N4CCCOCC4)cc3)c(=O)oc2c1] | 416.43 | ~1 |
| DDL-476 | 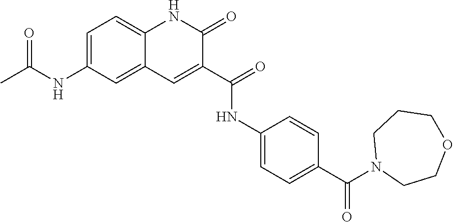 [SMILES: CC(=O)Nc1ccc2[nH]c(=O)c(C(=O)Nc3ccc(C(=O)N4CCCOCC4)cc3)cc2c1] | 448.48 | ~1 |
| DDL-477 | 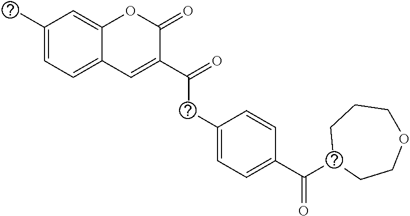 [SMILES: Cc1ccc2cc(C(=O)Cc3ccc(C(=O)N4CCCOCC4)cc3)c(=O)oc2c1] | 410.4 | ~1 |
| DDL-478 | 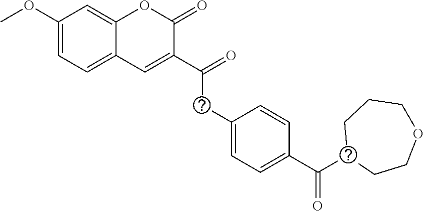 [SMILES: COc1ccc2cc(C(=O)Cc3ccc(C(=O)N4CCCOCC4)cc3)c(=O)oc2c1] | 422.4 | 1.1 |
| DDL-479 | 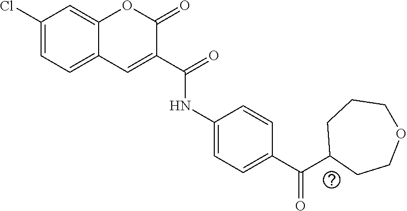 [SMILES: O=C(Nc1ccc(C(=O)C2CCCOCC2)cc1)c1cc2ccc(Cl)cc2oc1=O] | 426.9 | ~0.9 |
| DDL-480 | 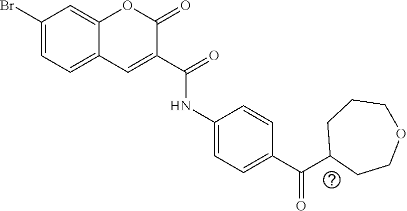 [SMILES: O=C(Nc1ccc(C(=O)C2CCCOCC2)cc1)c1cc2ccc(Br)cc2oc1=O] | 471.31 | ~0.9 |
| DDL-481 | 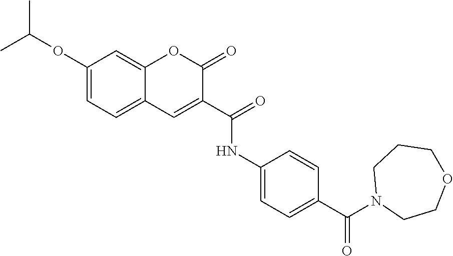 [SMILES: CC(C)Oc1ccc2cc(C(=O)Nc3ccc(C(=O)N4CCCOCC4)cc3)c(=O)oc2c1] | 450.49 | ~0.9 |
| DDL-482 | 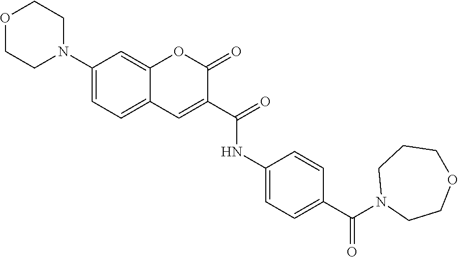 [SMILES: O=C(Nc1ccc(C(=O)N2CCCOCC2)cc1)c1cc2ccc(N3CCOCC3)cc2oc1=O] | 477.19 | ~1 |
| DDL-483 | 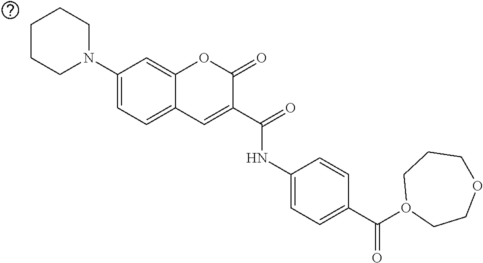 | 476.21 | ~1 |
| DDL-484 | 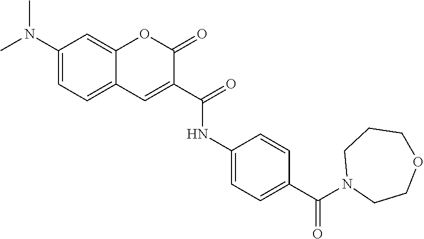 [SMILES: CN(C)c1ccc2cc(C(=O)Nc3ccc(C(=O)N4CCCOCC4)cc3)c(=O)oc2c1] | 435.18 | 1.1 |
| DDL-485 | 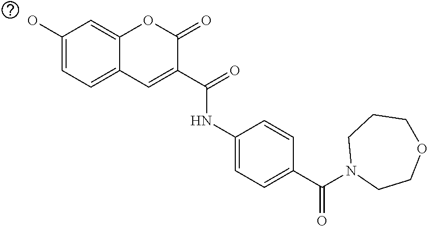 [SMILES: O=C(Nc1ccc(C(=O)N2CCCOCC2)cc1)c1cc2ccc(O)cc2oc1=O] | 408.41 | N/A* |
| DDL-486 | 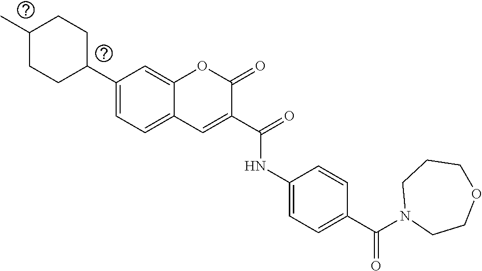 [SMILES: CC1CCC(c2ccc3cc(C(=O)Nc4ccc(C(=O)N5CCCOCC5)cc4)c(=O)oc3c2)CC1] | 490.56 | ~1 |
| DDL-487 | 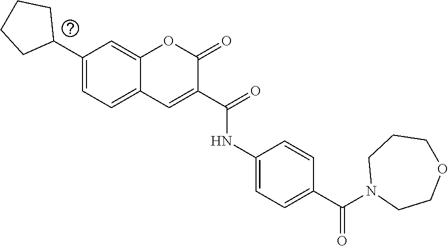 [SMILES: O=C(Nc1ccc(C(=O)N2CCCOCC2)cc1)c1cc2ccc(C3CCCC3)cc2oc1=O] | 461.83 | ~1 |
| DDL-488 | 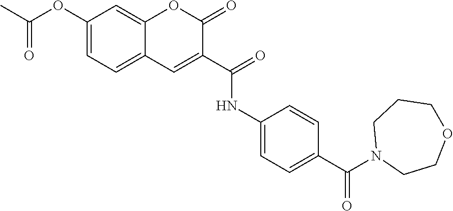 [SMILES: CC(=O)Oc1ccc2cc(C(=O)Nc3ccc(C(=O)N4CCCOCC4)cc3)c(=O)oc2c1] | 450.45 | N/A |
| DDL-489 | 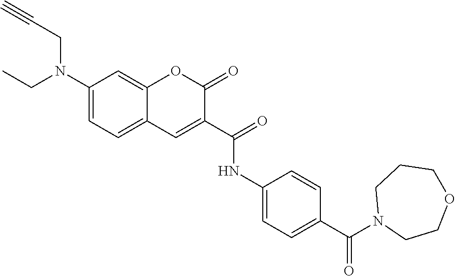 [SMILES: C#CCN(CC)c1ccc2cc(C(=O)Nc3ccc(C(=O)N4CCCOCC4)cc3)c(=O)oc2c1] | 473.53 | 1.2 |
| DDL-490 | 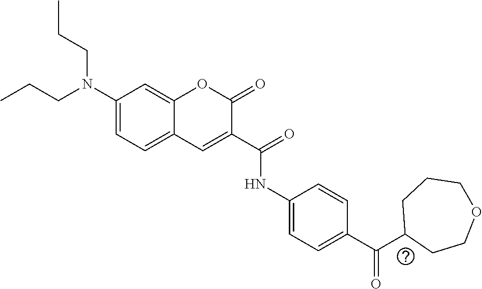 [SMILES: CCCN(CCC)c1ccc2cc(C(=O)Nc3ccc(C(=O)C4CCCOCC4)cc3)c(=O)oc2c1] | 491.59 | 1.2 |
| DDL-491 | 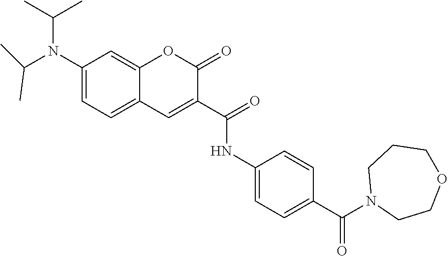 [SMILES: CC(C)N(c1ccc2cc(C(=O)Nc3ccc(C(=O)N4CCCOCC4)cc3)c(=O)oc2c1)C(C)C] | 491.59 | 1.4 |
| DDL-497 | 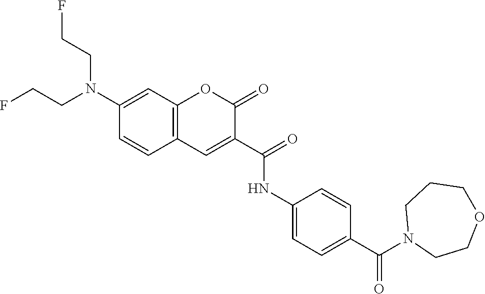 [SMILES: O=C(Nc1ccc(C(=O)N2CCCOCC2)cc1)c1cc2ccc(N(CCF)CCF)cc2oc1=O] | 499.51 | N/A |
| DDL-761 | 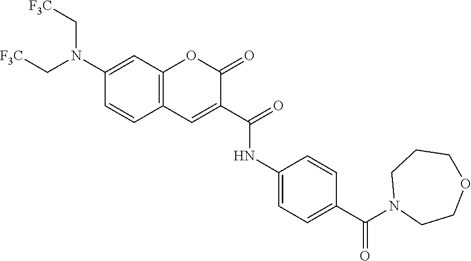 [SMILES: O=C(Nc1ccc(C(=O)N2CCCOCC2)cc1)c1cc2ccc(N(CC(F)(F)F)CC(F)(F)F)cc2oc1=O] | 571.48 | N/A |
| DDL-768 | 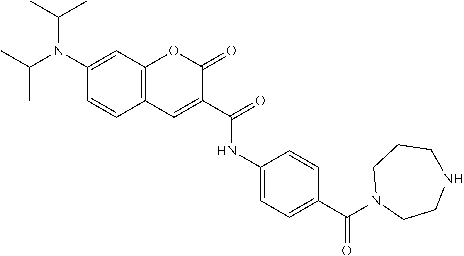 [SMILES: CC(C)N(c1ccc2cc(C(=O)Nc3ccc(C(=O)N4CCCNCC4)cc3)c(=O)oc2c1)C(C)C] | 490.60 | N/A |
| DDL-769 | 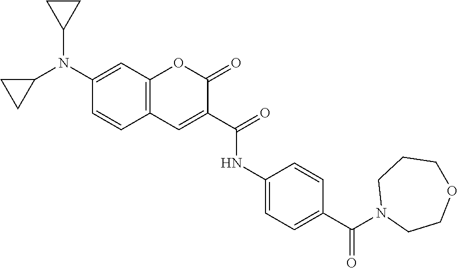 [SMILES: O=C(Nc1ccc(C(=O)N2CCCOCC2)cc1)c1cc2ccc(N(C3CC3)C3CC3)cc2oc1=O] | 487.56 | N/A |

As a summary, DDL-449 is able to increase SSPN transcript in skeletal muscle and heart of mdx mice up to 2.0-fold compared to vehicle-treated animals. DDL-472 increases SSPN transcript in skeletal muscle of mdx mice 1.5-2.0-fold. Both compounds rescue sarcolemma localization of SSPN, utrophin and β-Integrin proteins in muscle cells of mdx mice.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound represented by formula (I) or a pharmaceutically acceptable salt thereof: 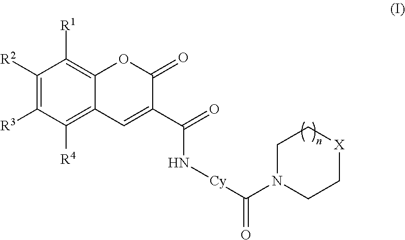
wherein
R¹, R², R³, and R⁴ are each independently H, alkyl, alkoxy, halo, nitrile, nitro, amino or aminoalkyl;
C_{y} is aryl or heteroaryl;
n is 1, or 2; and,
X is O or NH

2. The compound of claim 1, wherein Cy is aryl.

3. The compound of claim 1 or 2, wherein the compound is represented by formula (Ia):

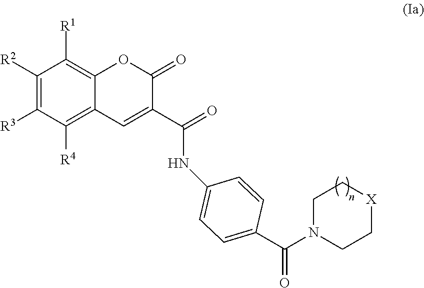

4. The compound of any one of claims 1-3, wherein R¹ and R⁴ are H.

5. The compound of any one of claims 1-4, wherein n is 2.

6. The compound of any one of claims 1-5, wherein X is O.

7. The compound of any one of claims 1-6, wherein R³ is H or halo.

8. The compound of any one of claims 1-7, wherein R³ is H.

9. The compound of any one of claims 1-8, wherein R² is H, halo, alkyl, alkoxy, amino, acetyl, or aminoalkyl.

10. The compound of any one of claims 1-9, wherein R² is a cyclic amine.

11. The compound of any one of claims 1-9, wherein R² is dimethylamino, diethylamino or di-isopropyl amino.

12. The compound of any one of claims 1-9, wherein R² is diethylamino.

13. The compound of any one of claims 1-12, wherein the compound is selected from: 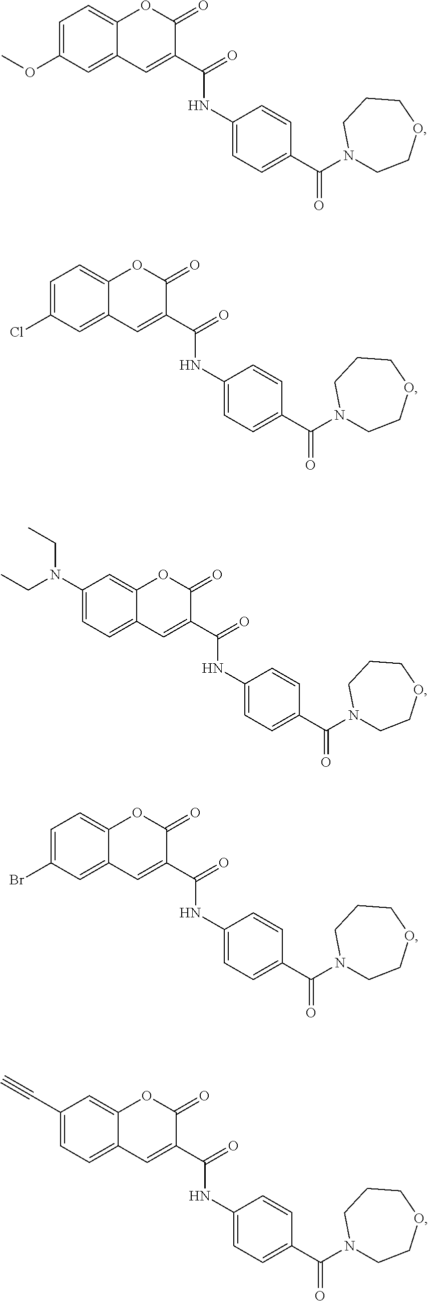 [SMILES: COc1ccc2oc(=O)c(C(=O)Nc3ccc(C(=O)N4CCCOCC4)cc3)cc2c1 ; O=C(Nc1ccc(C(=O)N2CCCOCC2)cc1)c1cc2cc(Cl)ccc2oc1=O ; CCN(CC)c1ccc2cc(C(=O)Nc3ccc(C(=O)N4CCCOCC4)cc3)c(=O)oc2c1 ; O=C(Nc1ccc(C(=O)N2CCCOCC2)cc1)c1cc2cc(Br)ccc2oc1=O ; C#Cc1ccc2cc(C(=O)Nc3ccc(C(=O)N4CCCOCC4)cc3)c(=O)oc2c1] 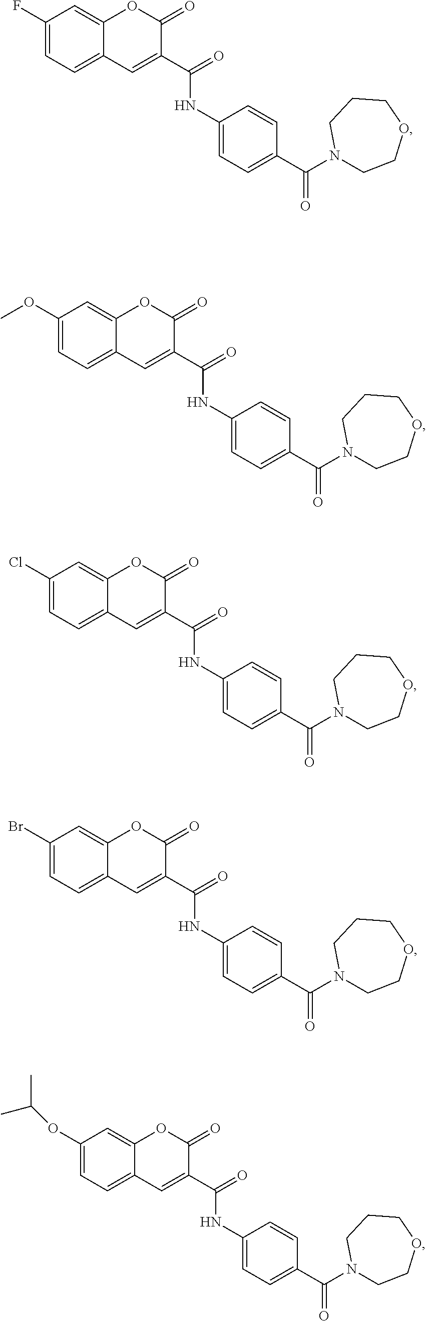 [SMILES: O=C(Nc1ccc(C(=O)N2CCCOCC2)cc1)c1cc2ccc(F)cc2oc1=O ; COc1ccc2cc(C(=O)Nc3ccc(C(=O)N4CCCOCC4)cc3)c(=O)oc2c1 ; O=C(Nc1ccc(C(=O)N2CCCOCC2)cc1)c1cc2ccc(Cl)cc2oc1=O ; O=C(Nc1ccc(C(=O)N2CCCOCC2)cc1)c1cc2ccc(Br)cc2oc1=O ; CC(C)Oc1ccc2cc(C(=O)Nc3ccc(C(=O)N4CCCOCC4)cc3)c(=O)oc2c1] 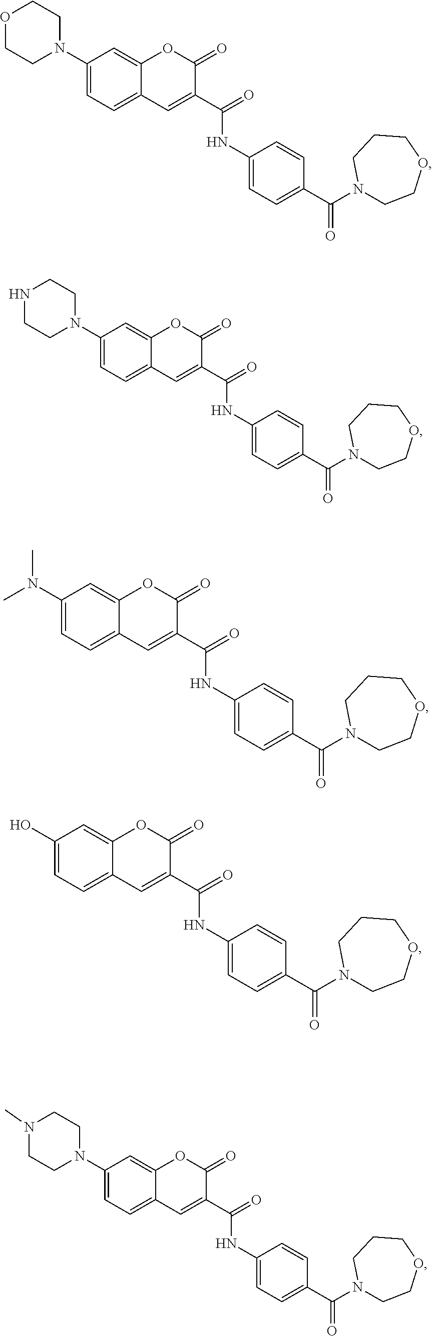 [SMILES: O=C(Nc1ccc(C(=O)N2CCCOCC2)cc1)c1cc2ccc(N3CCOCC3)cc2oc1=O ; O=C(Nc1ccc(C(=O)N2CCCOCC2)cc1)c1cc2ccc(N3CCNCC3)cc2oc1=O ; CN(C)c1ccc2cc(C(=O)Nc3ccc(C(=O)N4CCCOCC4)cc3)c(=O)oc2c1 ; O=C(Nc1ccc(C(=O)N2CCCOCC2)cc1)c1cc2ccc(O)cc2oc1=O ; CN1CCN(c2ccc3cc(C(=O)Nc4ccc(C(=O)N5CCCOCC5)cc4)c(=O)oc3c2)CC1] 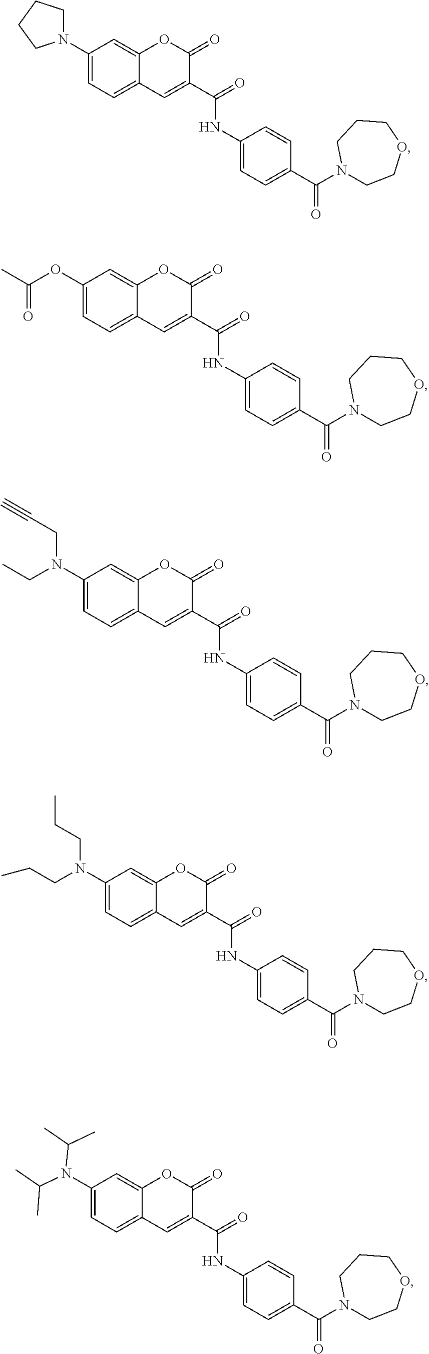 [SMILES: O=C(Nc1ccc(C(=O)N2CCCOCC2)cc1)c1cc2ccc(N3CCCC3)cc2oc1=O ; CC(=O)Oc1ccc2cc(C(=O)Nc3ccc(C(=O)N4CCCOCC4)cc3)c(=O)oc2c1 ; C#CCN(CC)c1ccc2cc(C(=O)Nc3ccc(C(=O)N4CCCOCC4)cc3)c(=O)oc2c1 ; CCCN(CCC)c1ccc2cc(C(=O)Nc3ccc(C(=O)N4CCCOCC4)cc3)c(=O)oc2c1 ; CC(C)N(c1ccc2cc(C(=O)Nc3ccc(C(=O)N4CCCOCC4)cc3)c(=O)oc2c1)C(C)C] 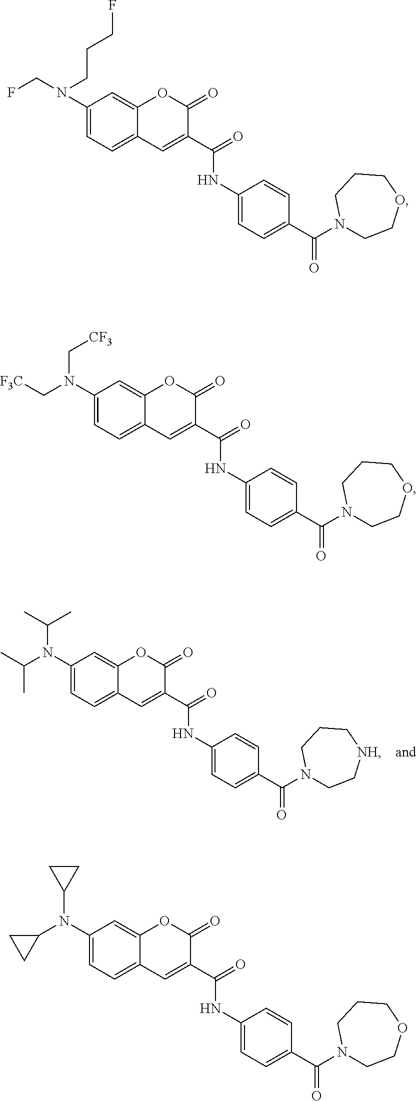 [SMILES: O=C(Nc1ccc(C(=O)N2CCCOCC2)cc1)c1cc2ccc(N(CF)CCCF)cc2oc1=O ; O=C(Nc1ccc(C(=O)N2CCCOCC2)cc1)c1cc2ccc(N(CC(F)(F)F)CC(F)(F)F)cc2oc1=O ; CC(C)N(c1ccc2cc(C(=O)Nc3ccc(C(=O)N4CCCNCC4)cc3)c(=O)oc2c1)C(C)C ; O=C(Nc1ccc(C(=O)N2CCCOCC2)cc1)c1cc2ccc(N(C3CC3)C3CC3)cc2oc1=O]
or a pharmaceutically acceptable salt thereof.

14. A compound represented by formula (II) or a pharmaceutically acceptable salt thereof: 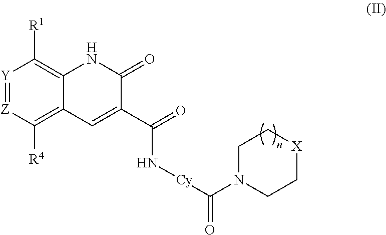
wherein
R¹ and R⁴ are each independently H, alkyl, alkoxy, halo, nitrile, nitro, amino or aminoalkyl;
Y is N or CR²;
R² is H, halo, nitro, amino, alkoxy, or alkyl;
Z is N or CR³;
R³ is H, halo, nitro, amino, alkoxy, or alkyl;
Cy is aryl or heteroaryl;
n is 1 or 2; and
X is O or NH; wherein the compound is not

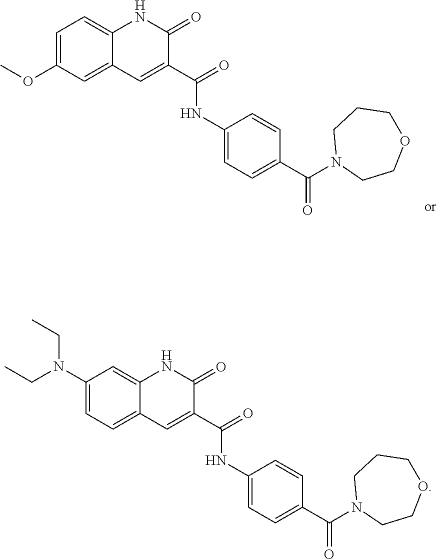 [SMILES: COc1ccc2[nH]c(=O)c(C(=O)Nc3ccc(C(=O)N4CCCOCC4)cc3)cc2c1 ; CCN(CC)c1ccc2cc(C(=O)Nc3ccc(C(=O)N4CCCOCC4)cc3)c(=O)[nH]c2c1]

15. The compound of claim 14, wherein Cy is aryl.

16. The compound of claim 14 or 15, wherein R¹ and R⁴ are H.

17. The compound of any one of claims 14-16, wherein n is 2.

18. The compound of any one of claims 14-17, wherein X is O.

19. The compound of any one of claims 14-18, wherein R³ is H, alkyl, alkoxy, halo, amino, or nitro.

20. The compound of any one of claims 14-19, wherein R³ is amino.

21. The compound of any one of claims 14-19, wherein R³ is haloalkyl.

22. The compound of any one of claims 14-19, wherein R³ is alkyl substituted with one or more F.

23. The compound of any one of claims 14-22, wherein Y is CR².

24. The compound of any one of claims 14-23, wherein R² is halo or alkoxy.

25. The compound of any one of claims 14-22, wherein Y is N.

26. The compound of any one of claims 14-25, wherein the compound is selected from: 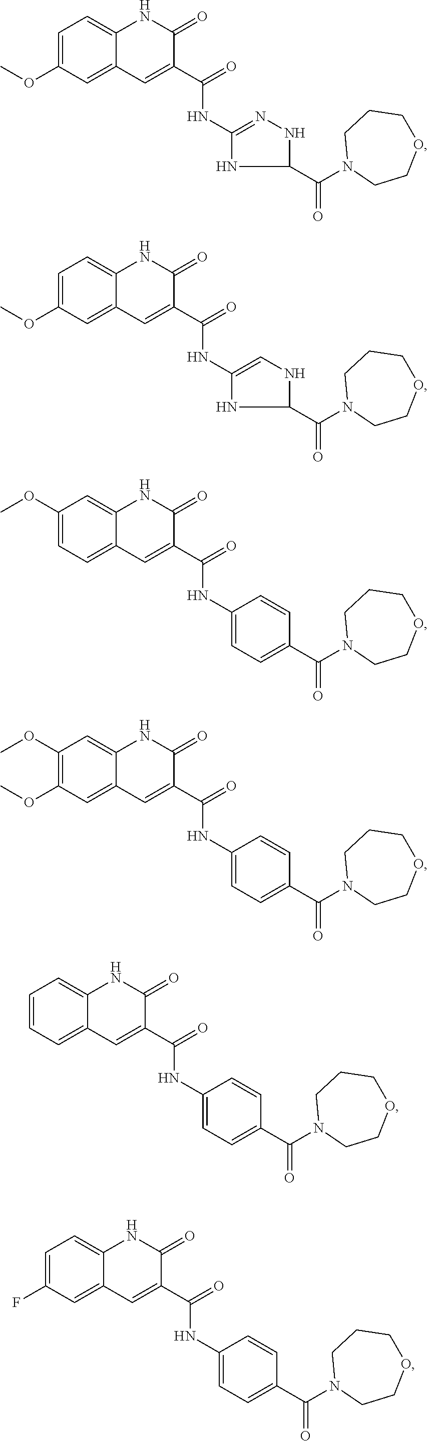 [SMILES: COc1ccc2[nH]c(=O)c(C(=O)NC3=NNC(C(=O)N4CCCOCC4)N3)cc2c1 ; COc1ccc2[nH]c(=O)c(C(=O)NC3=CNC(C(=O)N4CCCOCC4)N3)cc2c1 ; COc1ccc2cc(C(=O)Nc3ccc(C(=O)N4CCCOCC4)cc3)c(=O)[nH]c2c1 ; COc1cc2cc(C(=O)Nc3ccc(C(=O)N4CCCOCC4)cc3)c(=O)[nH]c2cc1OC ; O=C(Nc1ccc(C(=O)N2CCCOCC2)cc1)c1cc2ccccc2[nH]c1=O ; O=C(Nc1ccc(C(=O)N2CCCOCC2)cc1)c1cc2cc(F)ccc2[nH]c1=O] 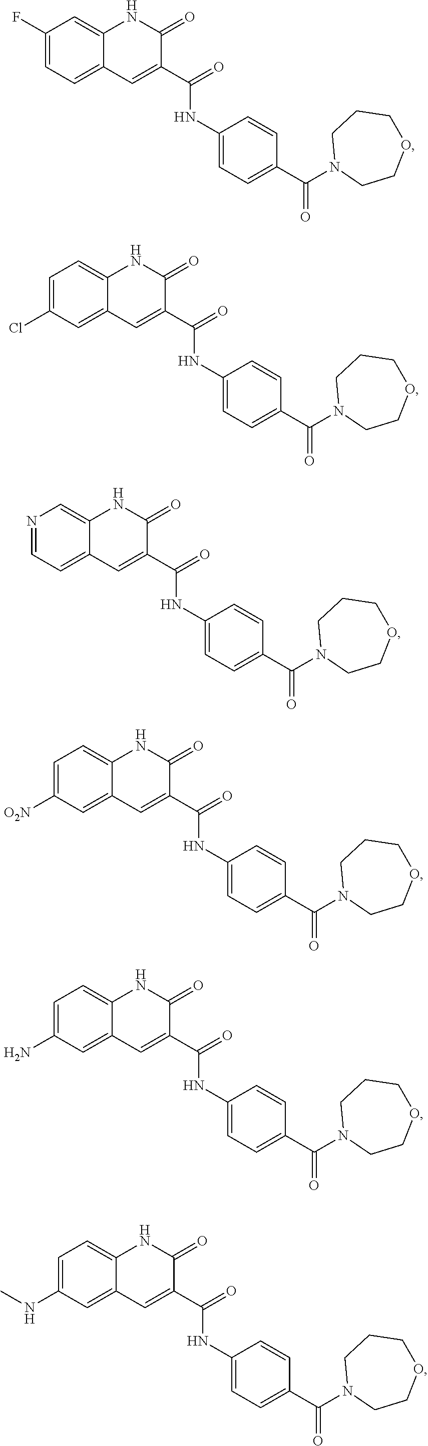 [SMILES: O=C(Nc1ccc(C(=O)N2CCCOCC2)cc1)c1cc2ccc(F)cc2[nH]c1=O ; O=C(Nc1ccc(C(=O)N2CCCOCC2)cc1)c1cc2cc(Cl)ccc2[nH]c1=O ; O=C(Nc1ccc(C(=O)N2CCCOCC2)cc1)c1cc2ccncc2[nH]c1=O ; O=C(Nc1ccc(C(=O)N2CCCOCC2)cc1)c1cc2cc([N+](=O)[O-])ccc2[nH]c1=O ; Nc1ccc2[nH]c(=O)c(C(=O)Nc3ccc(C(=O)N4CCCOCC4)cc3)cc2c1 ; CNc1ccc2[nH]c(=O)c(C(=O)Nc3ccc(C(=O)N4CCCOCC4)cc3)cc2c1] 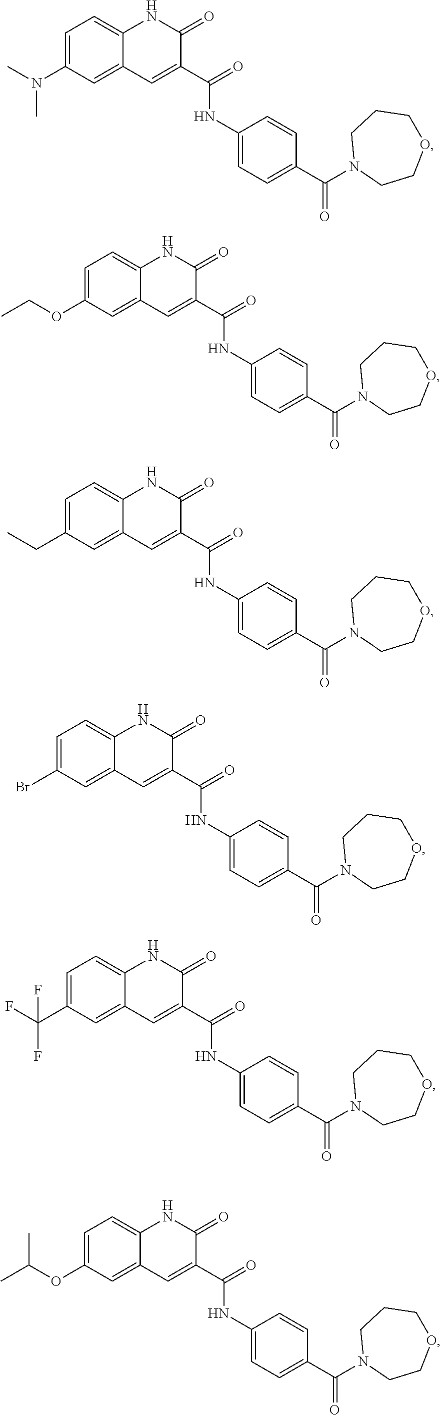 [SMILES: CN(C)c1ccc2[nH]c(=O)c(C(=O)Nc3ccc(C(=O)N4CCCOCC4)cc3)cc2c1 ; CCOc1ccc2[nH]c(=O)c(C(=O)Nc3ccc(C(=O)N4CCCOCC4)cc3)cc2c1 ; CCc1ccc2[nH]c(=O)c(C(=O)Nc3ccc(C(=O)N4CCCOCC4)cc3)cc2c1 ; O=C(Nc1ccc(C(=O)N2CCCOCC2)cc1)c1cc2cc(Br)ccc2[nH]c1=O ; O=C(Nc1ccc(C(=O)N2CCCOCC2)cc1)c1cc2cc(C(F)(F)F)ccc2[nH]c1=O ; CC(C)Oc1ccc2[nH]c(=O)c(C(=O)Nc3ccc(C(=O)N4CCCOCC4)cc3)cc2c1] 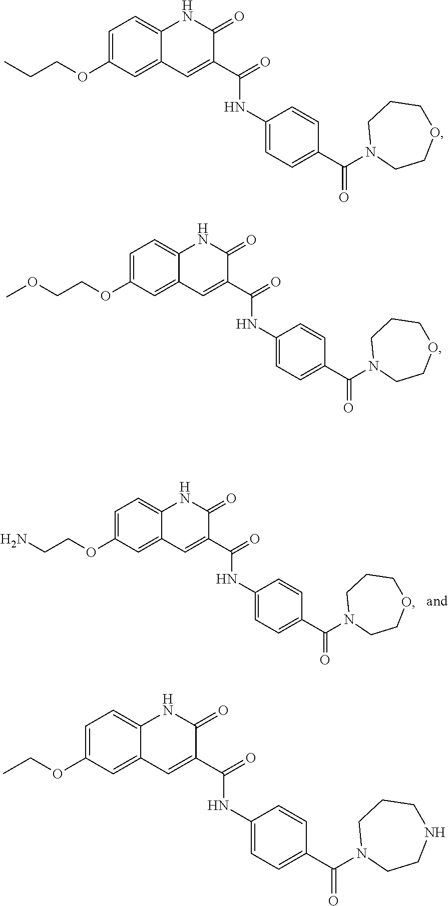 [SMILES: CCCOc1ccc2[nH]c(=O)c(C(=O)Nc3ccc(C(=O)N4CCCOCC4)cc3)cc2c1 ; COCCOc1ccc2[nH]c(=O)c(C(=O)Nc3ccc(C(=O)N4CCCOCC4)cc3)cc2c1 ; NCCOc1ccc2[nH]c(=O)c(C(=O)Nc3ccc(C(=O)N4CCCOCC4)cc3)cc2c1 ; CCOc1ccc2[nH]c(=O)c(C(=O)Nc3ccc(C(=O)N4CCCNCC4)cc3)cc2c1]
or a pharmaceutically acceptable salt thereof.

27. A pharmaceutical composition comprising a compound of any one of claims 1-26 and a pharmaceutically available excipient.

28. A method of treating or preventing a disease related to dysfunction of a dystrophin-related complex in a subject in need thereof, comprising administering to the subject a compound of any one of claims 1-26.

29. The method of claim 28, wherein the disease related to dysfunction of a dystrophin-related complex is a muscular dystrophy.

30. The method of claim 28 or 29, wherein the disease is Duchenne muscular dystrophy.
